SEQUENCE LISTING (163 sequences)

SEQ ID NO: 1 - RNA, 59 nt, synthetic construct
  misc_feature 1..59: note = Synthetic: Partial gRNA sequence, artificial
  source 1..59: mol_type = other RNA
gaccaccccaaaaaatgaaggggactaaaacacaaatctatctgaataaactcttcttc

SEQ ID NO: 2 - RNA, 30 nt, synthetic construct
  misc_feature 1..30: note = Synthetic: Target RNA sequence, artificial
  source 1..30: mol_type = other RNA
ggaagaagagtttattcagatagatttgtc

SEQ ID NO: 3 - RNA, 52 nt, synthetic construct
  misc_feature 1..52: note = Synthetic: Partial gRNA sequence, artificial
  source 1..52: mol_type = other RNA
accccaaaaaatgaaggggactaaaactagattgctgttctaccagtaatcc

SEQ ID NO: 4 - RNA, 34 nt, synthetic construct
  misc_feature 1..34: note = Synthetic: Target RNA sequence, artificial
  source 1..34: mol_type = other RNA
ggattaactggtagaacagcaatctagttttagt

SEQ ID NO: 5 - RNA, 31 nt, synthetic construct
  misc_feature 1..31: note = Synthetic: Partial gRNA sequence, artificial
  source 1..31: mol_type = other RNA
ggaccaccccaaaaatgaaggggactaaaac

SEQ ID NO: 6 - RNA, 39 nt, synthetic construct
  misc_feature 1..39: note = Synthetic: Partial gRNA sequence, artificial
  source 1..39: mol_type = other RNA
ggaccaccccaaaaatgaaggggactaaaacacaaatct

SEQ ID NO: 7 - RNA, 16 nt, synthetic construct
  misc_feature 1..16: note = Synthetic: Target RNA sequence, artificial
  source 1..16: mol_type = other RNA
agatttgtgttttagt

SEQ ID NO: 8 - RNA, 50 nt, synthetic construct
  misc_feature 1..49: note = Synthetic: Partial gRNA sequence, artificial
  source 1..50: mol_type = other RNA
ggccaccccaaaaatgaaggggactaaaacacaaatntatctgaataaac

SEQ ID NO: 9 - RNA, 28 nt, synthetic construct
  misc_feature 1..27: note = Synthetic: Target RNA sequence, artificial
  source 1..28: mol_type = other RNA
gtttattcagatanatttgtcacagcag

SEQ ID NO: 10 - RNA, 50 nt, synthetic construct
  misc_feature 1..50: note = Synthetic: Partial gRNA sequence, artificial
  source 1..50: mol_type = other RNA
ggccaccccaaaaatgaaggggactaaaacataagcccatctaaataaat

SEQ ID NO: 11 - RNA, 28 nt, synthetic construct
  misc_feature 1..28: note = Synthetic: Target RNA sequence, artificial
  source 1..28: mol_type = other RNA
atttatttagatgggcttatcacagcag

SEQ ID NO: 12 - RNA, 50 nt, synthetic construct
  misc_feature 1..50: note = Synthetic: Partial gRNA sequence, artificial
  source 1..50: mol_type = other RNA
ggccaccccaaaaatgaaggggactaaaacgcaaatctatccgagtgagc

SEQ ID NO: 13 - RNA, 28 nt, synthetic construct
  misc_feature 1..28: note = Synthetic: Target RNA sequence, artificial
  source 1..28: mol_type = other RNA
gctcactcggatagatttgccacagcag

SEQ ID NO: 14 - protein, 1159 aa, Leptotrichia buccalis
  REGION 1..1159: note = misc_feature - Wild-type LbuCas13a amino acid sequence, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 15 - protein, 1159 aa, synthetic construct
  REGION 1..1159: note = Synthetic: Variant LbuCas13a R337A, amino acid sequence, artificial
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLANIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 16 - protein, 1159 aa, synthetic construct
  REGION 1..1159: note = Synthetic: Variant LbuCas13a N378A amino acid sequence, artificial
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRAIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 17 - protein, 1159 aa, synthetic construct
  REGION 1..1159: note = Synthetic: Variant LbuCas13a R963A amino acid sequence, artificial
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHALVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 18 - protein, 1159 aa, synthetic construct
  REGION 1..1159: note = Synthetic: Variant LbuCas13a R973A amino acid sequence, artificial
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWEADLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 19 - RNA, 31 nt, synthetic construct; identical to SEQ ID NO: 5
  misc_feature 1..31: note = Synthetic: Wild-type sequence of the handle region of LbuCas 13a guide RNA, synthetic
  source 1..31: mol_type = other RNA

SEQ ID NO: 20 - RNA, 30 nt, synthetic construct
  misc_feature 1..30: note = Synthetic: Mutated sequence of the handle region of LbuCas 13a guide RNA, synthetic
  source 1..30: mol_type = other RNA
ggccaccccaaaaatgaaggggactaaaac

SEQ ID NO: 21 - DNA, 28 bp, synthetic construct
  misc_feature 1..28: note = Synthetic: AM78_Lbu_R377A_ATH_fwd
  source 1..28: mol_type = other DNA
aagcgtttcttgccaacatcattggggt

SEQ ID NO: 22 - DNA, 28 bp, synthetic construct
  misc_feature 1..28: note = Synthetic: AM79_Lbu_N378A_ATH_fwd
  source 1..28: mol_type = other DNA
aagcgtttcttcgcgccatcattggggt

SEQ ID NO: 23 - DNA, 22 bp, synthetic construct
  misc_feature 1..22: note = Synthetic: AM80_Lbu_377_378_ATH_rev
  source 1..22: mol_type = other DNA
cattctgacggttgcgggcgat

SEQ ID NO: 24 - DNA, 26 bp, synthetic construct
  misc_feature 1..26: note = Synthetic: AM89_Lbu_R973A_ATH_rev
  source 1..26: mol_type = other DNA
gaagcgcagatcggcttcccaaattg

SEQ ID NO: 25 - DNA, 31 bp, synthetic construct
  misc_feature 1..31: note = Synthetic: AM90_Lbu_R973A_ATH_fwd
  source 1..31: mol_type = other DNA
cgccttaaaggtgagttcccagaaaaccaat

SEQ ID NO: 26 - DNA, 26 bp, synthetic construct
  misc_feature 1..26: note = Synthetic: AM85_Lbu_973_seq_fwd
  source 1..26: mol_type = other DNA
aggactatgaaagttacaagcaagct

SEQ ID NO: 27 - DNA, 23 bp, synthetic construct
  misc_feature 1..23: note = Synthetic: AM86_Lbu_377_378_seq_fwd
  source 1..23: mol_type = other DNA
ttgacacgtacgtccgtaattgt

SEQ ID NO: 28 - DNA, 23 bp, synthetic construct
  misc_feature 1..23: note = Synthetic: MOC-626_T7_opt
  source 1..23: mol_type = other DNA
ggcgtaatacgactcactatagg

SEQ ID NO: 29 - DNA, 77 bp, synthetic construct
  misc_feature 1..77: note = Synthetic: MOC-1226_longer_AM_Liu_Lbu_target_IVTtemp
  source 1..77: mol_type = other DNA
gtgtgggcttctgctgtgacaaatctatctgaataaactcttcttcttggtttccctatagtgagtcgtattacgcc

SEQ ID NO: 30 - DNA, 77 bp, synthetic construct
  misc_feature 1..77: note = Synthetic: MOC-1227_longer_AM_Liu_Lbu_antitag_IVTtemp
  source 1..77: mol_type = other DNA
gtgtgggcttactaaaacacaaatctatctgaataaactcttcttcttggtttccctatagtgagtcgtattacgcc

SEQ ID NO: 31 - DNA, 78 bp, synthetic construct
  misc_feature 1..78: note = Synthetic: AM91_Lbu_Liu_target_MM25:28
  source 1..78: mol_type = other DNA
gtgtgggcttctgctgtggacaaatctatctgaataaactcttgaagttggtttccctatagtgagtcgtattacgcc

SEQ ID NO: 32 - DNA, 78 bp, synthetic construct
  misc_feature 1..78: note = Synthetic: AM92_Lbu_Liu_target_MM21:24
  source 1..78: mol_type = other DNA
gtgtgggcttctgctgtggacaaatctatctgaataaacagaacttcttggtttccctatagtgagtcgtattacgcc

SEQ ID NO: 33 - DNA, 78 bp, synthetic construct
  misc_feature 1..78: note = Synthetic: AM93_Lbu_Liu_target_MM17:20
  source 1..78: mol_type = other DNA
gtgtgggcttctgctgtggacaaatctatctgaattttgtcttcttcttggtttccctatagtgagtcgtattacgcc

SEQ ID NO: 34 - DNA, 78 bp, synthetic construct
  misc_feature 1..78: note = Synthetic: AM94_Lbu_Liu_target_MM13:16
  source 1..78: mol_type = other DNA
gtgtgggcttctgctgtggacaaatctatctcttaaaactcttcttcttggtttccctatagtgagtcgtattacgcc

SEQ ID NO: 35 - DNA, 78 bp, synthetic construct
  misc_feature 1..78: note = Synthetic: AM95_Lbu_Liu_target_MM9:12
  source 1..78: mol_type = other DNA
gtgtgggcttctgctgtggacaaatcttagagaataaactcttcttcttggtttccctatagtgagtcgtattacgcc

SEQ ID NO: 36 - DNA, 78 bp, synthetic construct
  misc_feature 1..78: note = Synthetic: AM96_Lbu_Liu_target_MM5:8
  source 1..78: mol_type = other DNA
gtgtgggcttctgctgtggacaatagaatctgaataaactcttcttcttggtttccctatagtgagtcgtattacgcc

SEQ ID NO: 37 - DNA, 78 bp, synthetic construct
  misc_feature 1..78: note = Synthetic: AM97_Lbu_Liu_target_MM1:4
  source 1..78: mol_type = other DNA
gtgtgggcttctgctgtggtgttatctatctgaataaactcttcttcttggtttccctatagtgagtcgtattacgcc

SEQ ID NO: 38 - DNA, 78 bp, synthetic construct
  misc_feature 1..78: note = Synthetic: AM108_Lbu_Liu_target_SM1
  source 1..78: mol_type = other DNA
gtgtgggcttctgctgtggtcaaatctatctgaataaactcttcttcttggtttccctatagtgagtcgtattacgcc

SEQ ID NO: 39 - DNA, 78 bp, synthetic construct
  misc_feature 1..78: note = Synthetic: AM109_Lbu_Liu_target_SM2
  source 1..78: mol_type = other DNA
gtgtgggcttctgctgtggagaaatctatctgaataaactcttcttcttggtttccctatagtgagtcgtattacgcc

SEQ ID NO: 40 - DNA, 78 bp, synthetic construct
  misc_feature 1..78: note = Synthetic: AM110_Lbu_Liu_target_SM3
  source 1..78: mol_type = other DNA
gtgtgggcttctgctgtggactaatctatctgaataaactcttcttcttggtttccctatagtgagtcgtattacgcc

SEQ ID NO: 41 - DNA, 78 bp, synthetic construct
  misc_feature 1..78: note = Synthetic: AM111_Lbu_Liu_target_SM4
  source 1..78: mol_type = other DNA
gtgtgggcttctgctgtggacatatctatctgaataaactcttcttcttggtttccctatagtgagtcgtattacgcc

SEQ ID NO: 42 - DNA, 78 bp, synthetic construct
  misc_feature 1..78: note = Synthetic: AM112_Lbu_Liu_target_SM5
  source 1..78: mol_type = other DNA
gtgtgggcttctgctgtggacaattctatctgaataaactcttcttcttggtttccctatagtgagtcgtattacgcc

SEQ ID NO: 43 - DNA, 78 bp, synthetic construct
  misc_feature 1..78: note = Synthetic: AM113_Lbu_Liu_target_SM6
  source 1..78: mol_type = other DNA
gtgtgggcttctgctgtggacaaaactatctgaataaactcttcttcttggtttccctatagtgagtcgtattacgcc

SEQ ID NO: 44 - DNA, 78 bp, synthetic construct
  misc_feature 1..78: note = Synthetic: AM114_Lbu_Liu_target_SM7
  source 1..78: mol_type = other DNA
gtgtgggcttctgctgtggacaaatgtatctgaataaactcttcttcttggtttccctatagtgagtcgtattacgcc

SEQ ID NO: 45 - DNA, 78 bp, synthetic construct
  misc_feature 1..78: note = Synthetic: AM115_Lbu_Liu_target_SM8
  source 1..78: mol_type = other DNA
gtgtgggcttctgctgtggacaaatcaatctgaataaactcttcttcttggtttccctatagtgagtcgtattacgcc

SEQ ID NO: 46 - DNA, 78 bp, synthetic construct
  misc_feature 1..78: note = Synthetic: AM116_Lbu_Liu_target_SM9
  source 1..78: mol_type = other DNA
gtgtgggcttctgctgtggacaaatctttctgaataaactcttcttcttggtttccctatagtgagtcgtattacgcc

SEQ ID NO: 47 - DNA, 78 bp, synthetic construct
  misc_feature 1..78: note = Synthetic: AM117_Lbu_Liu_target_SM10
  source 1..78: mol_type = other DNA
gtgtgggcttctgctgtggacaaatctaactgaataaactcttcttcttggtttccctatagtgagtcgtattacgcc

SEQ ID NO: 48 - DNA, 78 bp, synthetic construct
  misc_feature 1..78: note = Synthetic: AM118_Lbu_Liu_target_SM11
  source 1..78: mol_type = other DNA
gtgtgggcttctgctgtggacaaatctatgtgaataaactcttcttcttggtttccctatagtgagtcgtattacgcc

SEQ ID NO: 49 - DNA, 78 bp, synthetic construct
  misc_feature 1..78: note = Synthetic: AM119_Lbu_Liu_target_SM12
  source 1..78: mol_type = other DNA
gtgtgggcttctgctgtggacaaatctatcagaataaactcttcttcttggtttccctatagtgagtcgtattacgcc

SEQ ID NO: 50 - DNA, 78 bp, synthetic construct
  misc_feature 1..78: note = Synthetic: AM120_Lbu_Liu_target_SM13
  source 1..78: mol_type = other DNA
gtgtgggcttctgctgtggacaaatctatctcaataaactcttcttcttggtttccctatagtgagtcgtattacgcc

SEQ ID NO: 51 - DNA, 78 bp, synthetic construct
  misc_feature 1..78: note = Synthetic: AM121_Lbu_Liu_target_SM14
  source 1..78: mol_type = other DNA
gtgtgggcttctgctgtggacaaatctatctgtataaactcttcttcttggtttccctatagtgagtcgtattacgcc

SEQ ID NO: 52 - DNA, 78 bp, synthetic construct
  misc_feature 1..78: note = Synthetic: AM122_Lbu_Liu_target_SM15
  source 1..78: mol_type = other DNA
gtgtgggcttctgctgtggacaaatctatctgattaaactcttcttcttggtttccctatagtgagtcgtattacgcc

SEQ ID NO: 53 - DNA, 78 bp, synthetic construct
  misc_feature 1..78: note = Synthetic: AM123_Lbu_Liu_target_SM16
  source 1..78: mol_type = other DNA
gtgtgggcttctgctgtggacaaatctatctgaaaaaactcttcttcttggtttccctatagtgagtcgtattacgcc

SEQ ID NO: 54 - DNA, 78 bp, synthetic construct
  misc_feature 1..78: note = Synthetic: AM124_Lbu_Liu_target_SM17
  source 1..78: mol_type = other DNA
gtgtgggcttctgctgtggacaaatctatctgaattaactcttcttcttggtttccctatagtgagtcgtattacgcc

SEQ ID NO: 55 - DNA, 78 bp, synthetic construct
  misc_feature 1..78: note = Synthetic: AM125_Lbu_Liu_target_SM18
  source 1..78: mol_type = other DNA
gtgtgggcttctgctgtggacaaatctatctgaatatactcttcttcttggtttccctatagtgagtcgtattacgcc

SEQ ID NO: 56 - DNA, 78 bp, synthetic construct
  misc_feature 1..78: note = Synthetic: AM126_Lbu_Liu_target_SM19
  source 1..78: mol_type = other DNA
gtgtgggcttctgctgtggacaaatctatctgaataatctcttcttcttggtttccctatagtgagtcgtattacgcc

SEQ ID NO: 57 - DNA, 78 bp, synthetic construct
  misc_feature 1..78: note = Synthetic: AM127_Lbu_Liu_target_SM20
  source 1..78: mol_type = other DNA
gtgtgggcttctgctgtggacaaatctatctgaataaagtcttcttcttggtttccctatagtgagtcgtattacgcc

SEQ ID NO: 58 - DNA, 78 bp, synthetic construct
  misc_feature 1..78: note = Synthetic: AM160_SARS_COV2_S_D80_WT
  source 1..78: mol_type = other DNA
cattaaatggtaggacagggttatcaaacctcttagtaccattggtcccagagaccctatagtgagtcgtattacgcc

SEQ ID NO: 59 - DNA, 78 bp, synthetic construct
  misc_feature 1..78: note = Synthetic: AM161_SARS_COV2_S_D80_BETA
  source 1..78: mol_type = other DNA
cattaaatggtaggacagggttagcaaacctcttagtaccattggtcccagagaccctatagtgagtcgtattacgcc

SEQ ID NO: 60 - DNA, 78 bp, synthetic construct
  misc_feature 1..78: note = Synthetic: AM164_SARS_COV2_S_L452_WT
  source 1..78: mol_type = other DNA
ttagacttcctaaacaatctatacaggtaattataattaccaccaaccttagaatcctatagtgagtcgtattacgcc

SEQ ID NO: 61 - DNA, 78 bp, synthetic construct
  misc_feature 1..78: note = Synthetic: AM165_SARS_COV2_S_L452_DELTA
  source 1..78: mol_type = other DNA
ttagacttcctaaacaatctataccggtaattataattaccaccaaccttagaatcctatagtgagtcgtattacgcc

SEQ ID NO: 62 - DNA, 78 bp, synthetic construct
  misc_feature 1..78: note = Synthetic: AM168_SARS_COV2_S_S477_WT
  source 1..78: mol_type = other DNA
aaaccttcaacaccattacaaggtgtgctaccggcctgatagatttcagttgaaacctatagtgagtcgtattacgcc

SEQ ID NO: 63 - DNA, 78 bp, synthetic construct
  misc_feature 1..78: note = Synthetic: AM169_SARS_COV2_S_S477_OMICRON
  source 1..78: mol_type = other DNA
aaaccttcaacaccattacaaggtttgttaccggcctgatagatttcagttgaaacctatagtgagtcgtattacgcc

SEQ ID NO: 64 - RNA, 59 nt, synthetic construct
  misc_feature 1..59: note = Synthetic: MOC-1264_Lbu_mat_crRNA_Liu_28nt
  source 1..59: mol_type = other RNA
ggaccaccccaaaaatgaaggggactaaaacacaaatctatctgaataaactcttcttc

SEQ ID NO: 65 - RNA, 51 nt, synthetic construct
  misc_feature 1..51: note = Synthetic: MOC-1265_Lbu_mat_crRNA_Liu_20nt
  source 1..51: mol_type = other RNA
ggaccaccccaaaaatgaaggggactaaaacacaaatctatctgaataaac

SEQ ID NO: 66 - RNA, 47 nt, synthetic construct
  misc_feature 1..47: note = Synthetic: MOC-1266_Lbu_mat_crRNA_Liu_16nt
  source 1..47: mol_type = other RNA
ggaccaccccaaaaatgaaggggactaaaacacaaatctatctgaat

SEQ ID NO: 67 - RNA, 58 nt, synthetic construct
  misc_feature 1..58: note = Synthetic: AM208_Liu_mut_DR_28
  source 1..58: mol_type = other RNA
ggccaccccaaaaatgaaggggactaaaacacaaatctatctgaataaactcttcttc

SEQ ID NO: 68 - RNA, 50 nt, synthetic construct
  misc_feature 1..50: note = Synthetic: AM211_Liu_mut_DR_Lbu_20
  source 1..50: mol_type = other RNA
ggccaccccaaaaatgaaggggactaaaacacaaatctatctgaataaac

SEQ ID NO: 69 - DNA, 56 bp, synthetic construct
  misc_feature 1..56: note = Synthetic: MOC-1284_Liu_long_target_IDT_RNA
  source 1..56: mol_type = other DNA
gggaaaccaagaagaagagtttattcagatagatttgtcacagcagaagcccacac

SEQ ID NO: 70 - DNA, 56 bp, synthetic construct
  misc_feature 1..56: note = Synthetic: MOC-1285_Liu_long_anti-target_IDT_RNA
  source 1..56: mol_type = other DNA
gggaaaccaagaagaagagtttattcagatagatttgtgttttagtaagcccacac

SEQ ID NO: 71 - RNA, 56 nt, synthetic construct
  misc_feature 1..56: note = Synthetic: Liu_Perfect_match_target_RNA
  source 1..56: mol_type = other RNA
gggaaaccaagaagaagagtttattcagatagatttgtcacagcagaagcccacac

SEQ ID NO: 72 - RNA, 56 nt, synthetic construct
  misc_feature 1..56: note = Synthetic: Liu_target_RNA_MM1-4
  source 1..56: mol_type = other RNA
gggaaaccaagaagaagagtttattcagatagataacacacagcagaagcccacac

SEQ ID NO: 73 - RNA, 56 nt, synthetic construct
  misc_feature 1..56: note = Synthetic: Liu_target_RNA_MM5-8
  source 1..56: mol_type = other RNA
gggaaaccaagaagaagagtttattcagattctattgtcacagcagaagcccacac

SEQ ID NO: 74 - RNA, 56 nt, synthetic construct
  misc_feature 1..56: note = Synthetic: Liu_target_RNA_MM9-12
  source 1..56: mol_type = other RNA
gggaaaccaagaagaagagtttattctctaagatttgtcacagcagaagcccacac

SEQ ID NO: 75 - RNA, 56 nt, synthetic construct
  misc_feature 1..56: note = Synthetic: Liu_target_RNA_MM13-16
  source 1..56: mol_type = other RNA
gggaaaccaagaagaagagttttaagagatagatttgtcacagcagaagcccacac

SEQ ID NO: 76 - RNA, 56 nt, synthetic construct
  misc_feature 1..56: note = Synthetic: Liu_target_RNA_MM17-20
  source 1..56: mol_type = other RNA
gggaaaccaagaagaagacaaaattcagatagatttgtcacagcagaagcccacac

SEQ ID NO: 77 - RNA, 56 nt, synthetic construct
  misc_feature 1..56: note = Synthetic: Liu_target_RNA_MM21-24
  source 1..56: mol_type = other RNA
gggaaaccaagaagttctgtttattcagatagatttgtcacagcagaagcccacac

SEQ ID NO: 78 - RNA, 56 nt, synthetic construct
  misc_feature 1..56: note = Synthetic: Liu_target_RNA_MM25-28
  source 1..56: mol_type = other RNA
gggaaaccaacttcaagagtttattcagatagatttgtcacagcagaagcccacac

SEQ ID NO: 79 - RNA, 56 nt, synthetic construct
  misc_feature 1..56: note = Synthetic: Liu_target_RNA_SM1
  source 1..56: mol_type = other RNA
gggaaaccaagaagaagagtttattcagatagatttgacacagcagaagcccacac

SEQ ID NO: 80 - RNA, 56 nt, synthetic construct
  misc_feature 1..56: note = Synthetic: Liu_target_RNA_SM2
  source 1..56: mol_type = other RNA
gggaaaccaagaagaagagtttattcagatagatttctcacagcagaagcccacac

SEQ ID NO: 81 - RNA, 56 nt, synthetic construct
  misc_feature 1..56: note = Synthetic: Liu_target_RNA_SM3
  source 1..56: mol_type = other RNA
gggaaaccaagaagaagagtttattcagatagattagtcacagcagaagcccacac

SEQ ID NO: 82 - RNA, 56 nt, synthetic construct
  misc_feature 1..56: note = Synthetic: Liu_target_RNA_SM4
  source 1..56: mol_type = other RNA
gggaaaccaagaagaagagtttattcagatagatatgtcacagcagaagcccacac

SEQ ID NO: 83 - RNA, 56 nt, synthetic construct
  misc_feature 1..56: note = Synthetic: Liu_target_RNA_SM5
  source 1..56: mol_type = other RNA
gggaaaccaagaagaagagtttattcagatagaattgtcacagcagaagcccacac

SEQ ID NO: 84 - RNA, 56 nt, synthetic construct
  misc_feature 1..56: note = Synthetic: Liu_target_RNA_SM6
  source 1..56: mol_type = other RNA
gggaaaccaagaagaagagtttattcagatagttttgtcacagcagaagcccacac

SEQ ID NO: 85 - RNA, 56 nt, synthetic construct
  misc_feature 1..56: note = Synthetic: Liu_target_RNA_SM7
  source 1..56: mol_type = other RNA
gggaaaccaagaagaagagtttattcagatacatttgtcacagcagaagcccacac

SEQ ID NO: 86 - RNA, 56 nt, synthetic construct
  misc_feature 1..56: note = Synthetic: Liu_target_RNA_SM8
  source 1..56: mol_type = other RNA
gggaaaccaagaagaagagtttattcagattgatttgtcacagcagaagcccacac

SEQ ID NO: 87 - RNA, 56 nt, synthetic construct
  misc_feature 1..56: note = Synthetic: Liu_target_RNA_SM9
  source 1..56: mol_type = other RNA
gggaaaccaagaagaagagtttattcagaaagatttgtcacagcagaagcccacac

SEQ ID NO: 88 - RNA, 56 nt, synthetic construct
  misc_feature 1..56: note = Synthetic: Liu_target_RNA_SM10
  source 1..56: mol_type = other RNA
gggaaaccaagaagaagagtttattcagttagatttgtcacagcagaagcccacac

SEQ ID NO: 89 - RNA, 56 nt, synthetic construct
  misc_feature 1..56: note = Synthetic: Liu_target_RNA_SM11
  source 1..56: mol_type = other RNA
gggaaaccaagaagaagagtttattcacatagatttgtcacagcagaagcccacac

SEQ ID NO: 90 - RNA, 56 nt, synthetic construct
  misc_feature 1..56: note = Synthetic: Liu_target_RNA_SM12
  source 1..56: mol_type = other RNA
gggaaaccaagaagaagagtttattctgatagatttgtcacagcagaagcccacac

SEQ ID NO: 91 - RNA, 56 nt, synthetic construct
  misc_feature 1..56: note = Synthetic: Liu_target_RNA_SM13
  source 1..56: mol_type = other RNA
gggaaaccaagaagaagagtttattgagatagatttgtcacagcagaagcccacac

SEQ ID NO: 92 - RNA, 56 nt, synthetic construct
  misc_feature 1..56: note = Synthetic: Liu_target_RNA_SM14
  source 1..56: mol_type = other RNA
gggaaaccaagaagaagagtttatacagatagatttgtcacagcagaagcccacac

SEQ ID NO: 93 - RNA, 56 nt, synthetic construct
  misc_feature 1..56: note = Synthetic: Liu_target_RNA_SM15
  source 1..56: mol_type = other RNA
gggaaaccaagaagaagagtttaatcagatagatttgtcacagcagaagcccacac

SEQ ID NO: 94 - RNA, 56 nt, synthetic construct
  misc_feature 1..56: note = Synthetic: Liu_target_RNA_SM16
  source 1..56: mol_type = other RNA
gggaaaccaagaagaagagttttttcagatagatttgtcacagcagaagcccacac

SEQ ID NO: 95 - RNA, 56 nt, synthetic construct
  misc_feature 1..56: note = Synthetic: Liu_target_RNA_SM17
  source 1..56: mol_type = other RNA
gggaaaccaagaagaagagttaattcagatagatttgtcacagcagaagcccacac

SEQ ID NO: 96 - RNA, 56 nt, synthetic construct
  misc_feature 1..56: note = Synthetic: Liu_target_RNA_SM18
  source 1..56: mol_type = other RNA
gggaaaccaagaagaagagtatattcagatagatttgtcacagcagaagcccacac

SEQ ID NO: 97 - RNA, 56 nt, synthetic construct
  misc_feature 1..56: note = Synthetic: Liu_target_RNA_SM19
  source 1..56: mol_type = other RNA
gggaaaccaagaagaagagattattcagatagatttgtcacagcagaagcccacac

SEQ ID NO: 98 - RNA, 56 nt, synthetic construct
  misc_feature 1..56: note = Synthetic: Liu_target_RNA_SM20
  source 1..56: mol_type = other RNA
gggaaaccaagaagaagactttattcagatagatttgtcacagcagaagcccacac

SEQ ID NO: 99 - DNA, 57 bp, synthetic construct
  misc_feature 1..57: note = Synthetic: AM160_SARS_COV2_S_D80_WT
  source 1..57: mol_type = other DNA
gggtctctgggaccaatggtactaagaggtttgataaccctgtcctaccatttaatg

SEQ ID NO: 100 - DNA, 57 bp, synthetic construct
  misc_feature 1..57: note = Synthetic: AM161_SARS_COV2_S_D80_BETA
  source 1..57: mol_type = other DNA
gggtctctgggaccaatggtactaagaggtttgctaaccctgtcctaccatttaatg

SEQ ID NO: 101 - DNA, 57 bp, synthetic construct
  misc_feature 1..57: note = Synthetic: AM164_SARS_COV2_S_L452_WT
  source 1..57: mol_type = other DNA
ggattctaaggttggtggtaattataattacctgtatagattgtttaggaagtctaa

SEQ ID NO: 102 - DNA, 57 bp, synthetic construct
  misc_feature 1..57: note = Synthetic: AM165_SARS_COV2_S_L452_DELTA
  source 1..57: mol_type = other DNA
ggattctaaggttggtggtaattataattaccggtatagattgtttaggaagtctaa

SEQ ID NO: 103 - DNA, 57 bp, synthetic construct
  misc_feature 1..57: note = Synthetic: AM168_SARS_COV2_S_S477_WT
  source 1..57: mol_type = other DNA
ggtttcaactgaaatctatcaggccggtagcacaccttgtaatggtgttgaaggttt

SEQ ID NO: 104 - DNA, 57 bp, synthetic construct
  misc_feature 1..57: note = Synthetic: AM169_SARS_COV2_S_S477_OMICRON
  source 1..57: mol_type = other DNA
ggtttcaactgaaatctatcaggccggtaacaaaccttgtaatggtgttgaaggttt

SEQ ID NO: 105 - RNA, 51 nt, synthetic construct
  misc_feature 1..51: note = Synthetic: AM162_WT_crRNA_S_D80_nt
  source 1..51: mol_type = other RNA
ggaccaccccaaaaatgaaggggactaaaacgggttatcaaacctcttagt

SEQ ID NO: 106 - RNA, 51 nt, synthetic construct
  misc_feature 1..51: note = Synthetic: AM163_beta_crRNA_S_D80_20nt
  source 1..51: mol_type = other RNA
ggaccaccccaaaaatgaaggggactaaaacgggttagcaaacctcttagt

SEQ ID NO: 107 - RNA, 51 nt, synthetic construct
  misc_feature 1..51: note = Synthetic: AM166_WT_crRNA_L452_S_20nt
  source 1..51: mol_type = other RNA
ggaccaccccaaaaatgaaggggactaaaacctatacaggtaattataatt

SEQ ID NO: 108 - RNA, 51 nt, synthetic construct
  misc_feature 1..51: note = Synthetic: AM167_Delta_crRNA_L452R_S_20nt
  source 1..51: mol_type = other RNA
ggaccaccccaaaaatgaaggggactaaaacctataccggtaattataatt

SEQ ID NO: 109 - RNA, 51 nt, synthetic construct
  misc_feature 1..51: note = Synthetic: AM170_WT_crRNA_S477_S_20nt
  source 1..51: mol_type = other RNA
ggaccaccccaaaaatgaaggggactaaaaccaaggtgtgctaccggcctg

SEQ ID NO: 110 - RNA, 51 nt, synthetic construct
  misc_feature 1..51: note = Synthetic: AM171_omicron_crRNA_477_478_S_20nt
  source 1..51: mol_type = other RNA
ggaccaccccaaaaatgaaggggactaaaaccaaggtttgttaccggcctg

SEQ ID NO: 111 - RNA, 51 nt, synthetic construct
  misc_feature 1..51: note = Synthetic: AM194_WT_crRNA_S_D80_20nt_MM19
  source 1..51: mol_type = other RNA
ggaccaccccaaaaatgaaggggactaaaacgggttatcaaacctcttact

SEQ ID NO: 112 - RNA, 51 nt, synthetic construct
  misc_feature 1..51: note = Synthetic: AM195_beta_crRNA_S_D80_20nt_MM7_19
  source 1..51: mol_type = other RNA
ggaccaccccaaaaatgaaggggactaaaacgggttagcaaacctcttact

SEQ ID NO: 113 - RNA, 51 nt, synthetic construct
  misc_feature 1..51: note = Synthetic: AM196_WT_crRNA_L452_S_20nt_MM19
  source 1..51: mol_type = other RNA
ggaccaccccaaaaatgaaggggactaaaacctatacaggtaattataaat

SEQ ID NO: 114 - RNA, 51 nt, synthetic construct
  misc_feature 1..51: note = Synthetic: AM197_Delta_crRNA_L452R_S_20nt_MM7_19
  source 1..51: mol_type = other RNA
ggaccaccccaaaaatgaaggggactaaaacctataccggtaattataaat

SEQ ID NO: 115 - RNA, 51 nt, synthetic construct
  misc_feature 1..51: note = Synthetic: AM198_WT_crRNA_S477_S_20nt_MM19
  source 1..51: mol_type = other RNA
ggaccaccccaaaaatgaaggggactaaaaccaaggtgtgctaccggccag

SEQ ID NO: 116 - RNA, 51 nt, synthetic construct
  misc_feature 1..51: note = Synthetic: AM199_omicron_crRNA_477_478_S_20nt_MM7_19
  source 1..51: mol_type = other RNA
ggaccaccccaaaaatgaaggggactaaaaccaaggtttgttaccggccag

SEQ ID NO: 117 - RNA, 51 nt, synthetic construct
  misc_feature 1..51: note = Synthetic: AM202_wt_crRNA_D80_v2
  source 1..51: mol_type = other RNA
ggaccaccccaaaaatgaaggggactaaaacaatggtaggacagggttatc

SEQ ID NO: 118 - RNA, 51 nt, synthetic construct
  misc_feature 1..51: note = Synthetic: AM203_beta_crRNA_D80_v2_MM19
  source 1..51: mol_type = other RNA
ggaccaccccaaaaatgaaggggactaaaacaatggtaggacagggttagc

SEQ ID NO: 119 - RNA, 51 nt, synthetic construct
  misc_feature 1..51: note = Synthetic: AM204_wt_crRNA_L452_v2
  source 1..51: mol_type = other RNA
ggaccaccccaaaaatgaaggggactaaaacttcctaaacaatctatacag

SEQ ID NO: 120 - RNA, 51 nt, synthetic construct
  misc_feature 1..51: note = Synthetic: AM205_delta_crRNA_L452_v2_MM19
  source 1..51: mol_type = other RNA
ggaccaccccaaaaatgaaggggactaaaacttcctaaacaatctataccg

SEQ ID NO: 121 - RNA, 51 nt, synthetic construct
  misc_feature 1..51: note = Synthetic: AM206_wt_crRNA_S477_v2
  source 1..51: mol_type = other RNA
ggaccaccccaaaaatgaaggggactaaaacacaccattacaaggtgtgct

SEQ ID NO: 122 - RNA, 51 nt, synthetic construct
  misc_feature 1..51: note = Synthetic: AM207_omicron_crRNA_S477_v2_MM19
  source 1..51: mol_type = other RNA
ggaccaccccaaaaatgaaggggactaaaacacaccattacaaggtttgtt

SEQ ID NO: 123 - RNA, 50 nt, synthetic construct
  misc_feature 1..50: note = Synthetic: AM212_WT_del_crRNA_S_D80_nt
  source 1..50: mol_type = other RNA
ggccaccccaaaaatgaaggggactaaaacgggttatcaaacctcttagt

SEQ ID NO: 124 - RNA, 50 nt, synthetic construct
  misc_feature 1..50: note = Synthetic: AM213_beta_del_crRNA_S_D80_20nt
  source 1..50: mol_type = other RNA
ggccaccccaaaaatgaaggggactaaaacgggttagcaaacctcttagt

SEQ ID NO: 125 - RNA, 50 nt, synthetic construct
  misc_feature 1..50: note = Synthetic: AM214_WT_del_crRNA_L452_S_20nt
  source 1..50: mol_type = other RNA
ggccaccccaaaaatgaaggggactaaaacctatacaggtaattataatt

SEQ ID NO: 126 - RNA, 50 nt, synthetic construct
  misc_feature 1..50: note = Synthetic: AM215_Delta_del_crRNA_L452R_S_20nt
  source 1..50: mol_type = other RNA
ggccaccccaaaaatgaaggggactaaaacctataccggtaattataatt

SEQ ID NO: 127 - RNA, 50 nt, synthetic construct
  misc_feature 1..50: note = Synthetic: AM216_WT_del_crRNA_S477_S_20nt
  source 1..50: mol_type = other RNA
ggccaccccaaaaatgaaggggactaaaaccaaggtgtgctaccggcctg

SEQ ID NO: 128 - RNA, 50 nt, synthetic construct
  misc_feature 1..50: note = Synthetic: AM217_omicron_del_crRNA_477_478_S_20nt
  source 1..50: mol_type = other RNA
ggccaccccaaaaatgaaggggactaaaaccaaggtttgttaccggcctg

SEQ ID NO: 129 - RNA, 50 nt, synthetic construct
  misc_feature 1..50: note = Synthetic: AM222_crRNA_S_D80_20nt_MM19_mut_DR
  source 1..50: mol_type = other RNA
ggccaccccaaaaatgaaggggactaaaacgggttatcaaacctcttact

SEQ ID NO: 130 - RNA, 50 nt, synthetic construct
  misc_feature 1..50: note = Synthetic: AM223_beta_crRNA_S_D80_20nt_MM7_19_mut_DR
  source 1..50: mol_type = other RNA
ggccaccccaaaaatgaaggggactaaaacgggttagcaaacctcttact

SEQ ID NO: 131 - RNA, 50 nt, synthetic construct
  misc_feature 1..50: note = Synthetic: AM224_WT_crRNA_L452_S_20nt_MM19_mut_DR
  source 1..50: mol_type = other RNA
ggccaccccaaaaatgaaggggactaaaacctatacaggtaattataaat

SEQ ID NO: 132 - RNA, 50 nt, synthetic construct
  misc_feature 1..50: note = Synthetic: AM225_Delta_crRNA_L452R_S_20nt_MM7_19_mut_DR
  source 1..50: mol_type = other RNA
ggccaccccaaaaatgaaggggactaaaacctataccggtaattataaat

SEQ ID NO: 133 - RNA, 50 nt, synthetic construct
  misc_feature 1..50: note = Synthetic: AM226_WT_crRNA_S477_S_20nt_MM19_mut_DR
  source 1..50: mol_type = other RNA
ggccaccccaaaaatgaaggggactaaaaccaaggtgtgctaccggccag

SEQ ID NO: 134 - RNA, 50 nt, synthetic construct
  misc_feature 1..50: note = Synthetic: AM227_omicron_crRNA_477_478_S_20nt_MM7_19_mut_DR
  source 1..50: mol_type = other RNA
ggccaccccaaaaatgaaggggactaaaaccaaggtttgttaccggccag

SEQ ID NO: 135 - RNA, 50 nt, synthetic construct
  misc_feature 1..50: note = Synthetic: AM228_Liu_mut_DR_Lbu_20_pos7_G
  source 1..50: mol_type = other RNA
ggccaccccaaaaatgaaggggactaaaacacaaatgtatctgaataaac

SEQ ID NO: 136 - RNA, 50 nt, synthetic construct
  misc_feature 1..50: note = Synthetic: AM229_Liu_mut_DR_Lbu_20_pos7_A
  source 1..50: mol_type = other RNA
ggccaccccaaaaatgaaggggactaaaacacaaatatatctgaataaac

SEQ ID NO: 137 - RNA, 50 nt, synthetic construct
  misc_feature 1..50: note = Synthetic: AM230_Liu_mut_DR_Lbu_20_pos7_U
  source 1..50: mol_type = other RNA
ggccaccccaaaaatgaaggggactaaaacacaaatttatctgaataaac

SEQ ID NO: 138 - RNA, 50 nt, synthetic construct
  misc_feature 1..50: note = Synthetic: AM243_WT_del_crRNA_S_D80_20nt_MM6
  source 1..50: mol_type = other RNA
ggccaccccaaaaatgaaggggactaaaacgggttttcaaacctcttagt

SEQ ID NO: 139 - RNA, 50 nt, synthetic construct
  misc_feature 1..50: note = Synthetic: AM244_beta_del_crRNA_S_D80_20nt_MM6
  source 1..50: mol_type = other RNA
ggccaccccaaaaatgaaggggactaaaacgggtttgcaaacctcttagt

SEQ ID NO: 140 - RNA, 50 nt, synthetic construct
  misc_feature 1..50: note = Synthetic: AM245_WT_del_crRNA_L452_S_20nt_MM6
  source 1..50: mol_type = other RNA
ggccaccccaaaaatgaaggggactaaaacctatagaggtaattataatt

SEQ ID NO: 141 - RNA, 50 nt, synthetic construct
  misc_feature 1..50: note = Synthetic: AM246_Delta_del_crRNA_L452R_S_20nt_MM6
  source 1..50: mol_type = other RNA
ggccaccccaaaaatgaaggggactaaaacctatagcggtaattataatt

SEQ ID NO: 142 - RNA, 50 nt, synthetic construct
  misc_feature 1..50: note = Synthetic: AM247_WT_del_crRNA_S477_S_20nt_MM6
  source 1..50: mol_type = other RNA
ggccaccccaaaaatgaaggggactaaaaccaaggagtgctaccggcctg

SEQ ID NO: 143 - RNA, 50 nt, synthetic construct
  misc_feature 1..50: note = Synthetic: AM248_omicron_del_crRNA_477_478_S_20nt_MM6
  source 1..50: mol_type = other RNA
ggccaccccaaaaatgaaggggactaaaaccaaggattgttaccggcctg

SEQ ID NO: 144 - RNA, 51 nt, synthetic construct
  misc_feature 1..51: note = Synthetic: AM249_WT_crRNA_S_D80_20nt_MM6_19
  source 1..51: mol_type = other RNA
ggaccaccccaaaaatgaaggggactaaaacgggttttcaaacctcttact

SEQ ID NO: 145 - RNA, 56 nt, synthetic construct
  misc_feature 1..56: note = Synthetic: Liu_modified_localGC_3_G
  source 1..56: mol_type = other RNA
gggaaaccaagaataagagctcactcggatacatttgccacagcagaagcccacac

SEQ ID NO: 146 - RNA, 56 nt, synthetic construct
  misc_feature 1..56: note = Synthetic: Liu_modified_localGC_3_C_MM
  source 1..56: mol_type = other RNA
gggaaaccaagaataagagctcactcggatagatttgccacagcagaagcccacac

SEQ ID NO: 147 - RNA, 56 nt, synthetic construct
  misc_feature 1..56: note = Synthetic: Liu_modified_totalGC_50_G
  source 1..56: mol_type = other RNA
gggaaaccaagaagaagaatttatttagatgcgcttatcacagcagaagcccacac

SEQ ID NO: 148 - RNA, 56 nt, synthetic construct
  misc_feature 1..56: note = Synthetic: Liu_modified_totalGC_50_C_MM
  source 1..56: mol_type = other RNA
gggaaaccaagaagaagaatttatttagatgggcttatcacagcagaagcccacac

SEQ ID NO: 149 - RNA, 50 nt, synthetic construct; identical to SEQ ID NO: 10
  misc_feature 1..50: note = Synthetic: AM254_Lbu_mut_DR_modified_localGC_3_C
  source 1..50: mol_type = other RNA

SEQ ID NO: 150 - RNA, 50 nt, synthetic construct
  misc_feature 1..50: note = Synthetic: AM255_Lbu_mut_DR_modified_localGC_3_G
  source 1..50: mol_type = other RNA
ggccaccccaaaaatgaaggggactaaaacataagcgcatctaaataaat

SEQ ID NO: 151 - RNA, 50 nt, synthetic construct; identical to SEQ ID NO: 12
  misc_feature 1..50: note = Synthetic: AM256_Lbu_mut_DR_modified_totalGC_50_C
  source 1..50: mol_type = other RNA

SEQ ID NO: 152 - RNA, 50 nt, synthetic construct
  misc_feature 1..50: note = Synthetic: AM257_Lbu_mut_DR_modified_totalGC_50_G
  source 1..50: mol_type = other RNA
ggccaccccaaaaatgaaggggactaaaacgcaaatgtatccgagtgagc

SEQ ID NO: 153 - RNA, 50 nt, synthetic construct
  misc_feature 1..50: note = Synthetic: AM261_WT_del_crRNA_L452R_S_20nt_MM19
  source 1..50: mol_type = other RNA
ggccaccccaaaaatgaaggggactaaaacttcctaaacaatctatacag

SEQ ID NO: 154 - RNA, 50 nt, synthetic construct
  misc_feature 1..50: note = Synthetic: AM262_Delta_del_crRNA_L452R_S_20nt_MM19
  source 1..50: mol_type = other RNA
ggccaccccaaaaatgaaggggactaaaacttcctaaacaatctataccg

SEQ ID NO: 155 - DNA, 57 bp, synthetic construct
  misc_feature 1..57: note = Synthetic: AM238_SARS_CoV2_D80A_fwd_Arizti-Sanz
  source 1..57: mol_type = other DNA
gaaattaatacgactcactatagggcaactcaggacttgttcttacctttcttttcc

SEQ ID NO: 156 - DNA, 26 bp, synthetic construct
  misc_feature 1..26: note = Synthetic: AM239_SARS_CoV2_D80A _rev_Arizti-Sanz
  source 1..26: mol_type = other DNA
aagcaaaataaacaccatcattaaat

SEQ ID NO: 157 - DNA, 55 bp, synthetic construct
  misc_feature 1..55: note = Synthetic: AM263_RPA_fwd_Yang_SARS_S452_T7
  source 1..55: mol_type = other DNA
gaaattaatacgactcactatagggcttgattctaaggttggtggtaattataat

SEQ ID NO: 158 - DNA, 30 bp, synthetic construct
  misc_feature 1..30: note = Synthetic: AM264_RPA_rev_Yang_SARS_S452
  source 1..30: mol_type = other DNA
aaggtttgagattagacttcctaaacaatc

SEQ ID NO: 159 - DNA, 55 bp, synthetic construct
  misc_feature 1..55: note = Synthetic: AM220_SARS_CoV2_T478K_fwd_Yang
  source 1..55: mol_type = other DNA
gaaattaatacgactcactatagggttgagagagatatttcaactgaaatctatc

SEQ ID NO: 160 - DNA, 30 bp, synthetic construct
  misc_feature 1..30: note = Synthetic: AM221_ SARS_CoV2_T478K_rev_Yang
  source 1..30: mol_type = other DNA
agtgggttggaaaccatatgattgtaaagg

SEQ ID NO: 161 - RNA, 59 nt, synthetic construct; identical to SEQ ID NO: 64
  misc_feature 1..59: note = Synthetic: crRNA-target RNA (tgRNA)
  source 1..59: mol_type = other RNA

SEQ ID NO: 162 - RNA, 36 nt, synthetic construct
  misc_feature 1..36: note = Synthetic: crRNA-anti-tag RNA (A)
  source 1..36: mol_type = other RNA
gaagaagagtttattcagatagatttgtcacagcag

SEQ ID NO: 163 - RNA, 36 nt, synthetic construct
  misc_feature 1..36: note = Synthetic: crRNA-anti-tag RNA (B)
  source 1..36: mol_type = other RNA
gaagaagagtttattcagatagatttgtgttttagt

(19) United States
(12) Patent Application Publication
(10) Pub. No.: US 2025/0368975 A1
(43) Pub. Date: Dec. 4, 2025

(54) COMPOSITIONS, METHODS AND SYSTEMS FOR HIGH-FIDELITY CAS13A VARIANTS WITH IMPROVED SPECIFICITY

(71) Applicants: University of Rochester, Rochester, NY (US); University of California, Riverside, Riverside, CA (US)

(72) Inventors: Mitchell O'CONNELL, Rochester, NY (US); Adrian Moises Molina VARGAS, Rochester, NY (US); Giulia PALERMO, Riverside, CA (US); Souvik SINHA, Riverside, CA (US); Pablo R. ARANTES, Riverside, CA (US); Amun PATEL, Riverside, CA (US)

(73) Assignees: University of Rochester, Rochester, NY (US); University of California, Riverside, Riverside, CA (US)

(21) Appl. No.: 19/107,377

(22) PCT Filed: Oct. 24, 2023
(86) PCT No.: PCT/US2023/077588

§ 371 Date: Feb. 27, 2025

Related U.S. Application Data
(60) Provisional application No. 63/381,165, filed on Oct. 27, 2022.

(51) Int. Cl. C12N 9/22; C12N 15/11; C12Q 1/6823

(52) U.S. Cl. CPC C12N 9/226; C12N 15/11; C12Q 1/6823; C12N 2310/20

(57) ABSTRACT

Cas protein variants with improved specificity against mismatches between the guide RNA and an RNA target of interest are described. Also described are novel in-silico strategies for designing high specificity Cas variants, a method of detecting a target RNA in a sample with the Cas protein variant, and a kit for detecting a target RNA with the method.

This application claims priority from U.S. Provisional Patent Application No. 63/381,165, filed Oct. 27, 2022, which is incorporated herein by reference.

This invention was made with government support under GM133462 and GM141329 awarded by National Institutes of Health, and 2144823 awarded by National Science Foundation. The government has certain rights in the invention.

### FIELD

The present disclosure relates to Cas protein variants having site mutations that modulate a specificity of the Cas protein against mismatches between a guide RNA and a target RNA and methods of use thereof.

### BACKGROUND

CRISPR (Clustered Regulatory Interspaced Short Palindromic Repeats) and their associated (Cas) proteins are RNA-guided prokaryotic adaptive immune systems that protect bacteria and archaea against invading genetic elements, and when optimally programmed, certain CRISPR-Cas complexes can act as an exceptional genome editing tool. Cas13a (formerly known as C2c2) is a recently discovered Cas protein that binds and cleaves RNA, a property crucial for devising RNA detection based diagnostic applications. Upon binding the target RNA complementary to the guide crRNA, Cas13a effector activates the two Higher Eukaryotes and Prokaryotes Nucleotide (HEPN) catalytic domains, which are characteristic of RNA nucleases, to cleave RNA non-specifically (cis-/trans-). This non-specific nuclease activity of Cas13a has been exploited for the development of a range of ultrasensitive RNA detection tools including but not limited to: SHERLOCK, CARMEN, SPRINT, etc. However, these Cas13-based technologies still exhibit high tolerance for mismatches between the guide-RNA and target RNA of interest, limiting their use for the detection of mutations (e.g. single nucleotide polymorphisms [SNPs]), that could be harnessed for genetic testing, detection of aberrant gene expression, cancer detection, or epidemiological surveillance of pathogen's strains of concern. In this regard, rational design of Cas13 variants with improved specificity is pivotal to Cas13 based programmable RNA detection and diagnostic applications.

### SUMMARY

One aspect of the present application relates to a variant of a Cas protein. The variant comprises one or more mutations at the HEPN1 and HEPN2 interface of the Cas protein, wherein the one or more mutations modulate a specificity of the Cas protein against mismatches between a guide RNA and a target RNA.

Another aspect of the present application relates to a protein-RNA complex that comprises a Cas protein variant of the present application, a guide RNA, and optionally a target RNA.

Another aspect of the present application relates to computationally finding functional hotspots for mutations at the HEPN1 and HEPN2 interface of the Cas protein via investigating allosteric communication relevant to functionality.

Another aspect of the present application relates to a host cell genetically modified with an expression vector of the present application.

Another aspect of the present application relates to a method of detecting a single stranded target RNA in a sample. The method comprises the steps of (1) contacting the sample with: (i) a Cas guide RNA that hybridizes with a single stranded target RNA, and (ii) a Cas protein variant of the present application, and (2) measuring a detectable signal produced by Cas-mediated RNA cleavage.

Another aspect of the present application relates to a target RNA detection kit that comprises a Cas protein variant of the present application, a guide RNA that hybridizes with the target RNA, and instructions for the use of the kit components in diagnostic tests.

### BRIEF DESCRIPTION OF DRAWINGS

FIG. 1 shows an overview of the Leptotrichia buccalis (Lbu) Cas13a bound to a crRNA. Panel A shows the protein domains of a Cas 13 protein in different colors. The HEPN1 (mauve) and HEPN2 (green) catalytic domains are shown in molecular surface. The crRNA (orange) is shown as ribbons. The crRNA (also referred to as the guide RNA or gRNA) comprises a spacer region that is complementary to a target region in the target RNA, and a conserved handle region that contains a hairpin structure that interacts with the Cas protein. Panel B is a schematic diagram of the crRNA (orange) and target RNA (tgRNA, magenta) in the ternary complex of Cas13a (i.e., tgRNA-Cas13a). Panel Cis a schematic diagram of the nucleic acids in the ternary complex of Cas13a including an extended tag-antitag (blue) complementarity between the crRNA and tgRNA (atgRNA-Cas13). PDB codes of the cryo-EM and X-ray structures used as a reference for molecular simulations are reported in brackets. Panel D. Protein sequence and domain color code.

FIG. 2, Panel A shows a schematic representation of the allosteric signals from the crRNA “seed” (nt. 9-14) and “switch” (nt. 5-8) regions to the HEPN1-2 catalytic core residues (R472, H1053, H477, R1048) over the noise, computed as the communication between all pairs of crRNA bases and Cas13a residues. Two black arrows are used to indicate the signal standing up over the noise, depicted using grey arrows. The Signal-to-Noise Ratio (SNR) is computed as the mean-variance ratio (E[ ]/var[ ]) of the signal over the noise (details in the Material and Methods). Panel B shows distribution of the signals from the crRNA “seed” (green) and “switch” (blue) regions to the catalytic core residues, plotted on the background of noise (grey) for crRNA-Cas13a, tgRNA-Cas13a, and atgRNA-Cas13a complexes.

FIG. 3, Panel A shows signalling pathways connecting the crRNA “switch” region to the HEPN1-2 catalytic residues (R472, H1053, H477, R1048) in the tgRNA-Cas13a complex. FIG. 3, Panel B shows signalling pathways connecting the crRNA “seed” region to the HEPN1-2 catalytic residues (R472, H1053, H477, R1048) in the tgRNA-Cas13a complex. Residues occurring in the top five optimal pathways of communication are plotted on the three-dimensional structure of CRISPR-Cas13a (upper panel). Occurrence of residues in the top 5 optimal paths are shown in the barplot (lower panel). Residues are plotted using spheres of different colors according to the region of interest: “switch” (blue), “seed” (green), catalytic residues (pink), and remaining path residues (red).

FIG. 4 shows an overview of the locations of crRNA “seed” and “switch” regions with respect to the HEPN1(I)-2 interface, and the catalytic core. Conformation of the A(−3) base (green) of the crRNA repeat region, in proximity to the HEPN1(I)-2 interface of the three RNA-bound complexes.

FIG. 5 shows: Panel A. Sankey plots reporting the frequency, f, of formed contacts between residues of the HEPN1(I) and HEPN2 domains, and the crRNA, forming for ≥10% of the simulation time (f>0.1) in the tgRNA-Cas13a. Residue pairs of HEPN1(I) (left), HEPN2 (right), and the crRNA bases (centre) are connected by edges whose width is proportional to f. Contact edges that involve A(−3) are shown in blue, to highlight them with respect to the remaining interactions (grey). Panel B. Close-up view of the HEPN1(I)-2 interface in the tgRNA-bound Cas13a. Panel C. Sankey plot reporting residue pairs at the HEPN1(I)-2 interface, which gain contact stability in either the crRNA-Cas13a (red) or the tgRNA-Cas13 (blue) to an extent of more than 10% with respect to the other. Panel D. Sankey plot reporting residue pairs of the HEPN1(I) (left), HEPN2 (right), and the crRNA bases (center), which gain stability in either the crRNA-Cas13a (red) or the tgRNA-Cas13 (blue) to an extent of more than 10% with respect to the other. Panel E. Sankey plots reporting the f, of formed contacts between residues of the HEPN1(I) and HEPN2 domains, and the crRNA where A(−3) is mutated to C(−3).

FIG. 6 shows: Panel A-Panel B. crRNA-target RNA (tgRNA) pair (Panel A) and crRNA-anti-tag RNA (atgRNA) pair (Panel B). Panel C. One-hour time course of background-corrected fluorescence measurements from RNA cleavage experiments by LbuCas13a WT and variants initiated by the addition of 100 pM tgRNA or atgRNA shown in (Panel A) and (Panel B). Panel D. Data from (Panel C) normalized as percent cleavage product generated relative to WT LbuCas13a with tgRNA.

FIG. 7 shows ratio between the Signal-to-Noise Ratio (SNRₘₐₓ) in the Cas13a variants and the WT Cas13a (SNRᵣₐₜᵢₒ=SNRᵥₐᵣᵢₐₙₜ/SNR_{wt}), computed for the tgRNA-(violet) and atgRNA-(mauve) bound systems.

FIG. 8 shows: Panel A. Close-up view of the HEPN1(I)-HEPN2 interface in the WT tgRNA-Cas13a (left panel) and its R963A mutant (right panel), showing the extrusion of A(−3) from the protein in the R963A mutant. Panel B. Polar plot of (i) the distance d between the Ca atom at position 963 and the center of mass of the N1-C6 ring (polar coordinate, in Å), and (ii) the dihedral angle θ between the C3′@A(−4), C3′@A(−3), C8@A(−4) and C2@A(−4) atoms (angular coordinate, in degrees), computed from the simulated ensembles of the WT tgRNA-bound Cas13a and its mutants. The ‘d’ distance and ‘θ’ angle are shown on the right.

FIG. 9 shows that LbuCas13a mutants show higher nuclease specificity with four mismatches between the crRNA and the target RNA. Panel A. Illustration of Cas13 RNA detection approach that harnesses the enzyme's trans-ssRNA cleavage activity for the cleavage of quenched fluorescent RNA reporters. Panel B. Experimental design of the regions for which four consecutive mismatches were introduced between the crRNA and the target RNA. Panel C. Heatmap that recapitulates the end-point fluorescent signal after 1 hour of Cas13a variants using 10 nM of target RNA, either with no mismatches (PM) or containing 4 consecutive mismatches in different regions as indicated. Results are background subtracted and normalized to values from WT Cas13a in the presence of PM RNA.

FIG. 10 shows heatmaps of normalized fluorescent signal after 1 hour of Cas13a variants using 10 nM of target RNA. Panel A. Relative cleavage efficiencies for each variant in the presence of a perfect match RNA (PM) or a single mismatch (SM) at different positions relative to the crRNA. Normalized to activity of wild-type Cas13a in the presence of PM RNA. Panel B-Panel D. Relative cleavage efficiencies for each Cas13a variant when activated with a SARS-COV-2 RNA fragment. crRNA and target were either designed for the ancestral/Wuhan strain or to a given VOC strain as follows: target A: beta, target B: delta, target C: omicron. The different designs are: Panel B. SNP occurs at position 7 relative to crRNA; Panel C. SNP occurs at position 19 relative to crRNA; Panel D. enzyme is primed with a mismatch at position 19 in all cases and SNP occurs at position 7 relative to crRNA.

FIG. 11 shows a comparison of cleavage performance of especially relevant variants and mismatch combinations across different SARS-COV-2 targets, depicted as the fluorescence ratio of that sample relative to the same assay conditions, crRNA and target using WT Cas13a enzyme. crRNA and target were either designed for the ancestral/Wuhan strain or to a given VOC strain as follows: target A: beta, target B: delta, target C: omicron. Panel A. Relative cleavage ratios for LbuCas13a^{R377A} where the position 7 of the crRNA is used for SNP detection. Panel B. Relative cleavage ratios for LbuCas13a^{R377A} where the position 19 of the crRNA is used for SNP detection. Panel C. Relative cleavage ratios for LbuCas13a^{N378A} where an initial synthetic mismatch is introduced at position 19 of the crRNA and position 7 is used for SNP detection.

FIG. 12 shows that Cas13 exhibits differential sensitivity to mismatches in a position-dependent manner and it is modulated by crRNA spacer length. Panel A: Schematic of a Cas13 RNA detection approach that harnesses the enzyme's trans-ssRNA (collateral) cleavage activity for the cleavage of quenched fluorescent RNA reporters. Panel B: Three different crRNA spacer lengths were used in the study of LbuCas13a nuclease activation: 16, 20 and 28 nucleotides. Panel C: LbuCas13a reporter cleavage time-course with different spacer lengths against the same target RNA with 100 PM target concentration. Panel D: Experimental design of the regions for which four consecutive mismatches (MM) were introduced between the crRNA and the target RNA. A perfect matched RNA is also used for reference (PM) Panel E: LbuCas13a relative reporter cleavage efficiency after 1 hour using different crRNA spacer lengths and mismatched target-RNAs at 100 pM (4 consecutive mismatches) Panel F: LbuCas13a relative reporter cleavage efficiency after 1 hour using different crRNA spacer lengths and mismatched target-RNAs at 10 nM (4 consecutive mismatches).

FIG. 13 shows mismatch-sensitive hotspots in Cas13. End-point (1 hour) LbuCas13a cleavage efficiencies of mismatch target-RNAs tiling at single nucleotide resolution across the target RNA compared to a perfect-matched (PM) ssRNA at two different target-RNA concentrations (10 nM and 100 pM) as follows: Panel A: a 28 nucleotide spacer and 10 nM target; Panel B: a 20 nucleotide spacer and 10 nM target; Panel C: a 28 nucleotide spacer and 100 PM target; Panel D: a 20 nucleotide spacer and 100 pM target.

FIG. 14 shows: Panel A. Distribution of the signals from the crRNA spacer regions to the catalytic core residues, plotted on the background of noise (grey), for perfectly matched (PM) and singly mismatched systems. Panel B. The ratio between the Signal-to-Noise Ratio (SNR) in the Cas13a: crRNA: target-RNA systems containing a PM target RNA and single mismatches at positions 4, 7, or 11; computed for spacer nt 1-4 and nt 5-8.

FIG. 15 shows LbuCas13a variants with higher reporter assay specificity against mismatches between the crRNA-spacer and the target RNA. Panel A: Heatmap of end-point fluorescence signal after 1 hour of LbuCas13a wild-type (WT) vs. variants using 10 nM of target RNA, either with no mismatches (PM) or 4 consecutive mismatches in the indicated regions. 28-nt. crRNA spacers were used. Results are background subtracted and normalized to values from WT LbuCas13a in the presence of PM RNA. Panels B-D: Relative cleavage efficiencies for each LbuCas13a variant in the presence of a perfect match RNA (PM) or a single-nucleotide mismatched RNA (SM) at different positions relative to the crRNA and at 10 nM concentration and 20-nt. crRNA-spacer. Cleavage efficiency was normalized to wild-type (WT) LbuCas13a in the presence of PM RNA for each of the studied variants as follows: Panel B, LbuCas13aR377A; Panel C, LbuCas13aN378A; and Panel D, LbuCas13aR973A.

FIG. 16 shows deletion in the crRNA direct repeat contributes to the partial inhibition by anti-tag containing RNAs and result in better discrimination of mismatch-containing RNAs. Panel A: Schematic of LbuCas13a crRNA sequence and structure. In red and pointed with an arrow, the adenine nucleotide that was deleted in Meeske and Marraffini (2018) and denoted as del crRNA. Panel B: Schematic of LbuCas13a crRNA structure when pairing with an anti-tag containing RNA. The extended 5′ 8-nucleotide complementarity with the direct repeat results in partial inhibition of LbuCas13a nuclease activity. In red and pointed with an arrow, the adenine nucleotide that was deleted in Meeske and Marraffini (2018) and denoted as del crRNA. Panel C: LbuCas13a reporter cleavage time-course with 20-nt. spacer of full-length (WT crRNA) or truncated crRNA (del crRNA) against the same target that contains an anti-tag (atgRNA) or not (tgRNA) with 100 pM or 10 nM final RNA target concentration as indicated. Panels D-F. Relative cleavage efficiencies for each LbuCas13a variant in the presence of a perfect match RNA (PM) or a single-nucleotide mismatched RNA (SM) at different positions relative to the crRNA and at 10 nM concentration. The crRNA used contain the adenine deletion in the direct repeat and the spacer is 20 nucleotides long. Cleavage efficiency was normalized to wild-type (WT) LbuCas13a in the presence of PM RNA for each of the studied variants as follows: Panel D. LbuCas13aR377A; Panel E. LbuCas13aN378A; and Panel F. LbuCas13aR973A

FIG. 17 shows that combining highly specific Cas13a variants and rational crRNA design strategies can be deployed for SNP detection of SARS-COV-2 strains. Panel A: SARS-COV-2 variants of concern and mutations relative to the ancestral strain assessed in this study. Panel B: Schematic of Cas13a-based detection of SNP mutations in SARS-COV-2 using a fluorescent readout with tailored crRNA designs; anc, ancestral; der, derived. Panel C: Schematic of a Cas13a RNA detection coupled to nucleic acid amplification. RNA is reverse transcribed into cDNA and T7 RNAP promoters are added during amplification. This amplified cDNA is used as template for T7 RNA polymerase transcription. The generated RNA products are detected by Cas13, and trans-cleavage of RNA reporters can be detected by either fluorescence or lateral flow, depending on the reporter design. Panel D: Comparison of one-hour end-point fluorescence signal from WT and LbuCas13a^{R973A} when detecting a SARS-COV-2 spike: D80S strain SNP (VOC target) or ancestral strain (anc. target) with a crRNA specific for the viral variant (VOC crRNA) or the ancestral virus (anc. crRNA). Panel E: Comparison of one-hour end-point fluorescence signal from WT when detecting a SARS-COV-2 spike: L452R strain SNP (VOC target) or ancestral strain (anc. target) with a crRNA specific for the viral variant (VOC crRNA) or the ancestral virus (anc. crRNA). Panel F: Comparison of one-hour end-point fluorescence signal from WT and LbuCas13a^{N378A} when detecting a SARS-COV-2 spike: S477N+T478K strain SNP (VOC target) or ancestral strain (anc. target) with a crRNA specific for the viral variant (VOC crRNA) or the ancestral virus (anc. crRNA).

FIG. 18 shows that the type of mismatch and local sequence context modulates Cas13-mismatch tolerance. Panel A: Schematic of crRNAs and target-RNAs used to study the specificity of LbuCas13a activation with all combinations of nucleotide base pairs at position 7 of the crRNA: target-RNA. Panel B: Heatmap showing the ratio of cleaved products after one hour incubation with 100 pM of a target with a given nucleotide base pair combination at position 7 compared to its corresponding canonical base pair. Panel C: Schematic of crRNA and target-RNA by increasing the GC content around position 7 of the crRNA: target-RNA but compensating across the duplex to maintain the original 25% GC overall content. Panel D: Schematic of crRNA and target-RNA by increasing the total GC content from 25 to 50% but maintaining the original sequence context around position 7 of the crRNA: target-RNA. Panel E: Comparison of one hour end-point fluorescence signal from LbuCas13a with 100 PM target, for the derived RNA sequences with different GC content, “high local GC content” corresponding to C and “high total GC content” corresponding to D. These measurements are performed with a perfectly-matched RNA target (PM) or containing a C-C mismatch at this position (MM).

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” are also disclosed, as appropriately understood by the skilled artisan. For example, if the value “10” is disclosed, the “less than or equal to 10” and “greater or equal to 10” is also disclosed. When two or more value are disclosed, all possible ranges between any two values are disclosed.

### I. Definitions

As used herein, the term “Cas protein” refers to a protein encoded by a Clustered Regularly Interspaced Short Palindromic Repeat-associated Protein (CRISPR) gene. Examples of Cas proteins include, but are not limited to, Cas3 proteins, Cas 5 proteins, Cas7 proteins, Cas8 proteins, Cas9 proteins, Cas10 proteins, Cas 12 proteins and Cas13 proteins. A Cas protein, when in complex with a suitable polynucleotide component, has endonuclease activity and is capable of recognizing, binding to, and optionally nicking, cleaving, or covalently attaching to all or part of a specific DNA or RNA target sequence.

As used herein, the terms “guide RNA, gRNA and crRNA” are used interchangeably and refer to an RNA molecule that a Cas protein binds and uses to identify a complementary RNA (the “target RNA” or DNA sequence).

As used herein, the term “Cas variant” (also “modified Cas protein” or “mutant Cas protein”) refers to Cas protein; such as, in some embodiments, a mammalian Cas13a protein created by human intervention. The Cas variant is a polypeptide having an altered amino acid sequence, relative to an unmodified or wild-type Cas protein. In some embodiments, the Cas variant is a polypeptide which differs from a wild-type Cas13a sequence by one or more amino acid substitutions, deletions, additions, or combinations thereof.

The term “wild-type” or “natural” or “native” as used herein is used in connection with biological materials such as nucleic acid molecules, proteins (e.g., Cas proteins) that are found in nature and not modified by human intervention.

The term “mismatch” refers to a nucleotide of a first polynucleotide that is not capable of pairing with a nucleotide at a corresponding position of a second polynucleotide, when the first and second polynucleotide are aligned.

The term “hybridization” refers to the pairing of complementary oligomeric compounds (e.g., an antisense oligonucleotide and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

The term “specifically hybridizes” refers to the ability of an oligomeric compound to hybridize to one nucleic acid site with greater affinity than it hybridizes to another nucleic acid site. In certain embodiments, an antisense oligonucleotide specifically hybridizes to more than one target site.

When in reference to nucleobases, the terms “nucleobase complementarity” and “complementarity” refer to a nucleobase that is capable of base pairing with another nucleobase. For example, in DNA, adenine (A) is complementary to thymine (T). For example, in RNA, adenine (A) is complementary to uracil (U). When used in reference to an oligonucleotide or portion thereof, the term “fully complementary” means that each nucleobase of the oligonucleotide or portion thereof is capable of pairing with a nucleobase of a complementary nucleic acid or contiguous portion thereof. Thus, a fully complementary region comprises no mismatches or unhybridized nucleobases in either strand. The term “partially complementary” means that one or more nucleobase of the oligonucleotide or portion thereof is not capable of pairing with the nucleobase(s) at the corresponding position(s) of a complementary nucleic acid or contiguous portion thereof.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “nucleotide sequence” includes all nucleotide sequences that are degenerate versions of each other and that encode the same amino acid sequence. Nucleotide sequences that encode proteins and RNA may include introns.

As used herein, the term “regulatory sequence” means a nucleic acid sequence which is required for expression of a coding sequence (either for protein or RNA) operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner. The term “promoter” as used herein is defined as a DNA sequence recognized by the synthetic machinery of the cell, or introduced synthetic machinery, required to initiate the specific transcription of a polynucleotide sequence. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which corresponds to the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

As used herein, the term “expression” is defined as the transcription and/or translation of a particular nucleotide sequence driven by a regulatory sequence such as a promoter and/or an enhancer.

As used herein, the term “expression vector” refers to a composition of matter which comprises a nucleotide sequence encoding a protein and/or an RNA and which can be used to deliver the nucleic acid sequence to the interior of a cell and express the encoded protein and/or RNA inside the cell. An expression vector typically comprises a regulatory sequence for expression of the protein or RNA encoded by the nucleotide sequence, wherein the regulatory sequence is operably linked to the nucleotide sequence. Expression vectors include non-viral vectors, such as plasmids, phagemids, and cosmids, and viral vectors, such as adenovirus vectors, adeno-associated virus (AAV) vectors, and retrovirus vectors.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

### II. Variants of Cas Proteins, Guide RNAs and Complexes Thereof

One aspect of the application relates to a variant of a Cas protein, comprising one or more mutations at the HEPN1 and HEPN2 interface of the Cas protein, wherein the one or more mutations modulate a specificity of the Cas protein against mismatches between a guide RNA and a target RNA.

The Cas protein can be any Cas protein that, when in complex with a suitable polynucleotide component, has endonuclease activity and is capable of recognizing, binding to, and cleaving a specific DNA or RNA target sequence. In some embodiments, the Cas protein is selected from the group consisting of Cas3 proteins, Cas 5 proteins, Cas7 proteins, Cas8 proteins, Cas9 proteins, Cas10 proteins, Cas 12 proteins and Cas13 proteins. In some embodiments, the Cas protein is a Cas13a protein. In some embodiments, the Cas protein is a Cas13a protein from Leptotrichia buccalis %% (LbuCas 13a). %

In some embodiments, the variant of the Cas protein of the present application has improved specificity, or reduced tolerance, to mismatches in a complementary region between a guide RNA and a target RNA, comparing to the corresponding wild-type Cas protein. Such improved specificity may be measured by methods well known in the art, such as the fluorescent ssRNA nuclease assays described in the present application, wherein improved specificity to a mismatch between the guide RNA and a target RNA is reflected by reduced trans-cleavage nuclease activity of the variant Cas protein/guide RNA/target RNA complex in the presence of the mismatch between the guide RNA and the target RNA. In some embodiments, the variant of the Cas protein of the present application has significantly improved specificity against a single nucleotide mismatch between the guide RNA and the target RNA, as compared to the corresponding wild-type Cas protein, wherein the significantly improved specificity is indicated by a decrease of at least 20%, 30%, 50%, 60%, 70%, 80%, or 90% of the trans-cleavage nuclease activity in a fluorescent ssRNA nuclease assays.

In some embodiments, the Cas protein variant of the present application is a variant of a Cas 13a protein, wherein the Cas protein variant comprises one or more amino acid mutations at positions corresponding to positions R377, N378, R963 and R973 of LbuCas 13a (SEQ ID NO:14). In some embodiments, the one or more mutations are substitutions. In some embodiments, the one or more mutations comprise an amino acid substitution correspond to the amino acid substitution of R377A in SEQ ID NO: 14. In some embodiments, the one or more mutations comprise an amino acid substitution correspond to the amino acid substitution of N378A in SEQ ID NO: 14. In some embodiments, the one or more mutations comprise an amino acid substitution correspond to the amino acid substitution of R963A in SEQ ID NO: 14. In some embodiments, the one or more mutations comprise an amino acid substitution correspond to the amino acid substitution of R973A in SEQ ID NO: 14.

As used hereinafter, the term “a position corresponding to position X of LbuCas 13a” refers to a position in the amino acid sequence of another Cas protein, wherein the amino acid residue at this position serves the same function in the three dimensional structure of the another Cas protein as the amino acid residue in position X of LbuCas 13a. Such determination is well known in the art and can be achieved with amino acid sequence alignment and three-dimensional structure modeling. For example, Cas13a from Leptotrichia Wadei (Lwa), a commonly used Cas13 ortholog, the amino acids R379, N380, R961 and R971 are conserved between and correspond to LbuCas13a residues R377, N378, R963, and R973, respectively.

In some embodiments, the Cas protein variant of the present application is a variant of the LbuCas 13a protein, wherein the Cas protein variant comprises one or more amino acid substitutions selected from the group consisting of R377A, N378A, R963A and R973A of SEQ ID NO:14.

In certain embodiments, the Cas protein variant has a protein sequence that is at least 80%, 85%, 90%, 95% or 98% homologous to SEQ ID NO:15.

In some embodiments, the Cas protein variant is LbuCas13aR377A (SEQ ID NO: 15).

In certain embodiments, the Cas protein variant has a protein sequence that is at least 80%, 85%, 90%, 95% or 98% homologous to SEQ ID NO:16.

In certain embodiments, the Cas protein variant is LbuCas13aN378A (SEQ ID NO: 16).

In certain embodiments, the Cas protein variant has a protein sequence that is at least 80%, 85%, 90%, 95% or 98% homologous to SEQ ID NO:17.

In certain embodiments, the Cas protein variant is LbuCas13aR963A (SEQ ID NO: 17).

In certain embodiments, the Cas protein variant has a protein sequence that is at least 80%, 85%, 90%, 95% or 98% homologous to SEQ ID NO:18.

In certain embodiments, the Cas protein variant is LbuCas13aR973A (SEQ ID NO: 18).

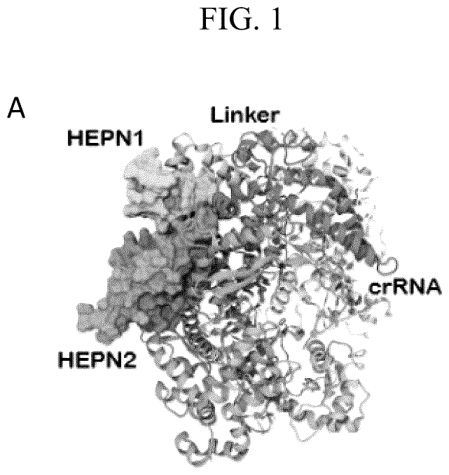

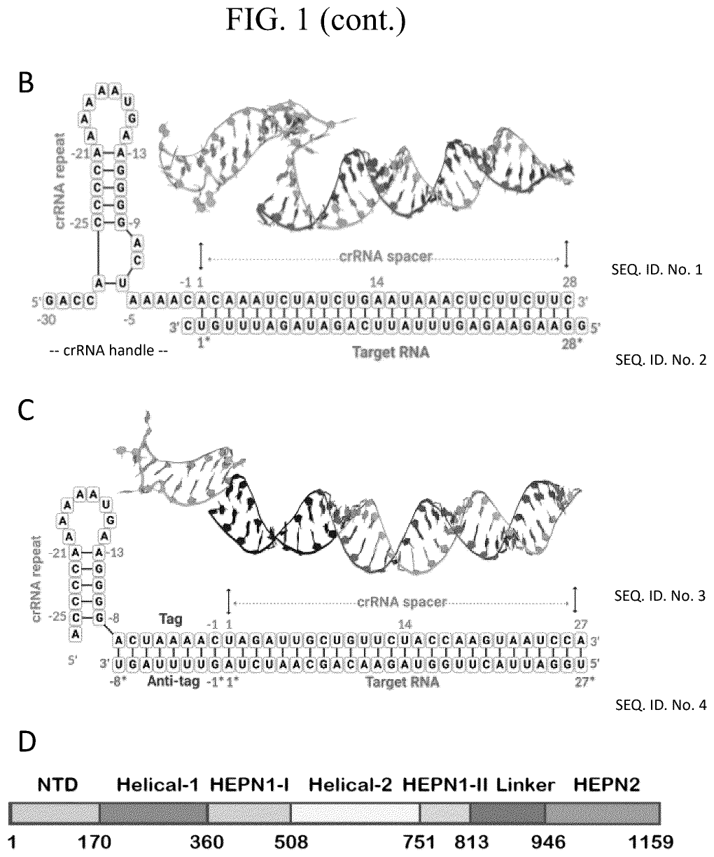

Another aspect of the present application relates to a guide RNA molecule that comprises a handle region and a spacer region. As shown in FIG. 1, Panel B, the handle region comprises a conserved hairpin structure that interacts with the Cas protein and the spacer region is complementary to a sequence in the target RNA. The handle region is directly linked to the spacer region, which is located at the 3′ end of the guide RNA.

In some embodiments, the spacer region has a length in the range of 10-50, 10-40, 10-30, 10-25, 10-20, 10-15, 12-50, 12-40, 12-30, 12-25, 12-20, 12-15, 15-50, 15-40, 15-30, 15-25, or 15-20 nt. In some embodiments, the spacer region has a length of 15-20 nt. In some embodiments, the spacer region has a length of about 20 nt.

In some embodiments, the spacer region has a length of 15-20 nt and contains one or more mismatches to a target RNA sequence. In some embodiments, the one or more mismatches are located in the last five nucleotides of the spacer region.

In some embodiments, the conserved hairpin structure has the sequence of SEQ ID NO:19 (5′-GGACCACCCCAAAAAUGAAGGGGACUAAAAC-3′, wild-type hairpin). In some embodiments, the handle region comprises one or more mutations in the conserved hairpin structure. In some embodiments, the conserved hairpin structure has the sequence of SEQ ID NO:20 (5′-GGCCACCCCAAAAAUGAAGGGGACUAAAAC-3′, mutated hairpin).

Another aspect of the application is a protein-RNA complex, comprising: a Cas protein variant as described herein; a guide RNA; and optionally a target RNA.

### III. Nucleotides, Expression Vectors and Host Cells

Another aspect of the present application relates to a nucleotide encoding a Cas protein variant as described herein and/or a guide RNA as described herein.

Another aspect of the present application relates to expression vector comprising a nucleic acid sequence encoding a Cas protein variant as described herein and/or a guide RNA as described herein; and a regulatory sequence operably linked to the nucleic acid sequence. Examples of regulatory sequence include, but are not limited to, promoters, enhancers, initiation sequences, transcription and translation terminators useful for regulation of the expression of the desired nucleic acid sequence.

The expression vectors can be any vector suitable for expression of the Cas protein variant and/or the guide RNA of the present application in eukaryotes. In some embodiments, the expression vector is a non-viral expression vector. In some embodiments, the non-viral expression vector is a plasmid vector, a cosmid vector or a phagemid vector.

In some embodiments, the expression vector is a viral expression vector. The term “viral expression vector” is used herein with reference to a virus that has been genetically altered, e.g., by the addition or insertion of a heterologous nucleic acid construct into a virus particle. Viral expression vectors may be derived from, e.g., adenoviruses, adeno-associated viruses (AAV), retroviruses (including lentiviruses, such as HIV-1 and HIV-2), vaccinia viruses and other poxviruses, herpesviruses (e.g., herpes simplex virus Types 1 and 2), polioviruses, Sindbis and other RNA viruses, alphaviruses, astroviruses, coronaviruses, orthomyxoviruses, papovaviruses, paramyxoviruses, parvoviruses, picornaviruses, togaviruses and others.

In some embodiments, the viral expression vectors may be engineered to target specific cells by using the targeting characteristics inherent to the virus vector or engineered into the viral expression vector. Specific cells may be “targeted” for delivery and expression of polynucleotides. Thus, “targeting,” in this case, relates to the use of endogenous or heterologous binding agents in the form of capsids, envelope proteins, antibodies for delivery to specific cells, the use of tissue-specific regulatory elements for restricting expression to specific subset(s) of cells, or both.

Another aspect of the application is a host cell that comprises an expression vector as described herein. In certain embodiments, the host cell is a eukaryotic cell. In certain embodiments, the host cell is a prokaryotic cell. In some embodiments, the expression vector is present in the host cell in an episomal form. In some embodiments, the expression vector is integrated into the hot cell genome.

### IV. Method of Making

Another aspect of the present application relates to a method of making the variant Cas protein of the present application. In some embodiments, the method comprises the step of introducing an expression vector comprising a nucleotide sequence encoding the variant Cas protein of the present application into a host cell, culturing the host cell for a desired period of time to allow expression of the variant Cas protein, and isolating the variant Cas protein from the host cell. In some embodiments, the method further comprises the step of generating an expression vector comprising a nucleotide sequence encoding the variant Cas protein of the present application.

In some embodiments, the nucleotide sequence encoding the variant Cas protein is generated from a nucleotide sequence encoding the corresponding wild-type Cas protein via site-directed mutagenesis. In some embodiments, the nucleotide sequence encoding the variant Cas protein is codon-optimized for more efficient expression.

### V. Method of Using

Another aspect of the present application relates to a method of detecting a target RNA in a sample.

In some embodiments, the method comprises the step of (A) contacting the sample with (i) a Cas guide RNA that comprises a spacer region that is complementary to a target region in the target RNA and (ii) a Cas protein variant as described herein; and (B) detecting a signal produced by Cas protein-mediated RNA cleavage. In some embodiments, the signal in step (B) is detected with a technology selected from the group consisting of gold nanoparticle based detection, fluorescence polarization, colloid phase transition/dispersion, electrochemical detection, and semiconductor-based sensing. In some embodiments, the target RNA is a SARS virus RNA.

Another aspect of the present application relates to a method of detecting a single stranded target RNA in a sample, wherein the target RNA contains a single nucleotide polymorphism (SNP) in a target region. The method comprises the steps of (1) contacting the sample with (i) a guide RNA comprising a handle region and a spacer region consisting of 15-40 nucleotides, wherein the spacer region is complementary to the target region of the target RNA, (ii) a Cas protein variant of the present application, and (iii) a reporting construct capable of producing a signal upon interacting with a Cas protein-RNA complex that has Cas nuclease activity, wherein the Cas protein-RNA complex comprises the guide RNA, the Cas protein variant and the target RNA; and (2) measuring the signal from the reporting construct, wherein detection of the signal from the reporting construct indicates the presence of the target RNA in the sample.

In some embodiments, the spacer region consists of 15-28 nucleotides.

In some embodiments, the spacer region consists of 15-20 nucleotides.

In some embodiments, the spacer region has a length determined based on the GC content of nucleotide sequences around the SNP.

In some embodiments, the handle region comprises the nucleotide sequence of SEQ ID NO: 19.

In some embodiments, the handle region comprises the nucleotide sequence of SEQ ID NO: 19 with one or more mutations.

In some embodiments, the handle region comprises the nucleotide sequence of SEQ ID NO:20.

In some embodiments, the target RNA is a SARS virus RNA.

### VI. Kits

Another aspect of the application is a target RNA detection kit comprising: a Cas protein variant as described herein; a guide RNA that hybridizes with the target RNA; and instructions for the use of said kit components in diagnostic tests.

In some embodiments, the kit further comprising a reporter construct or a reporter device that is capable of producing a detectable signal indicating the trans-cleavage nuclease activity from a Cas protein complex comprising the Cas protein variant the guide RNA and the target RNA.

The present application is further illustrated by the following examples that should not be construed as limiting. The contents of all references, patents, and published patent applications cited throughout this application, as well as the Figures and Tables, are incorporated herein by reference.

### EXAMPLES

### Example 1. Material and Methods

### Cas13a Protein Expression and Purification

For expression of wild-type LbuCas13a we used a plasmid that contains a codon-optimized Cas13a sequence which is N-terminally tagged with a His6-MBP-TEV-protease cleavage site sequence. (Addgene Plasmid #83482, East-Seletsky et al. (Nature, 538, 270-273 (2016))). LbuCas13a variants were generated from the wild-type vector via site-directed mutagenesis using the primers indicated in Table 1 below.

TABLE 1
Oligonucleotides used for site-directed mutagenesis (SEQ ID
NOS: 21-27)

| No. | Name | Sequence | Use |
|---|---|---|---|
| 21 | AM78_Lbu_R377A_ATH_fwd | AAGCGTTTCTTGCCAACATCATTGGGGT | Site- |
| | | | directed |
| | | | mutagenesis |
| 22 | AM79_Lbu_N378A_ATH_fwd | AAGCGTTTCTTCGCGCCATCATTGGGGT | Site- |
| | | | directed |
| | | | mutagenesis |
| 23 | AM80_Lbu_377_378_ATH_rev | CATTCTGACGGTTGCGGGCGAT | Site- |
| | | | directed |
| | | | mutagenesis- |
| | | | rev for |
| | | | both AM78 |
| | | | and AM79 |
| 24 | AM89_Lbu_R973A_ATH_rev | GAAGCGCAGATCGGCTTCCCAAATTG | Site- |
| | | | directed |
| | | | mutagenesis |
| 25 | AM90_Lbu_R973A_ATH_fwd | CGCCTTAAAGGTGAGTTCCCAGAAAACCAAT | Site- |
| | | | directed |
| | | | mutagenesis |
| 26 | AM85_Lbu_973_seq_fwd | AGGACTATGAAAGTTACAAGCAAGCT | Sanger |
| | | | sequencing |
| | | | primer |
| 27 | AM86_Lbu_377_378_seq_fwd | TTGACACGTACGTCCGTAATTGT | Sanger |
| | | | sequencing |
| | | | primer |

Purification of all constructs was carried out as previously described, with some modifications (Mol Cell, 66, 373-383 e373 (2017); Nature, 538, 270-273 (2016)). Briefly, expression vectors were transformed into Rosetta2 DE3 grown in LB media supplemented with 0.5% w/v glucose at 37° C. Protein expression was induced at mid-log phase (OD600˜0.6) with 0.5 mM IPTG, followed by incubation at 16° C. overnight. Cell pellets were resuspended in lysis buffer (50 mM HEPES [pH 7.0], 1 M NaCl, 5 mM imidazole, 5% (v/v) glycerol, 1 mM DTT, 0.5 mM PMSF, EDTA-free protease inhibitor [Roche]), lysed by sonication, and clarified by centrifugation at 15,000 g. Soluble His6-MBP-TEV-Cas13a was isolated over metal ion affinity chromatography, and in order to cleave off the His6-MBP tag, the protein-containing eluate was incubated with TEV protease at 4° C. overnight while dialyzing into ion exchange buffer (50 mM HEPES [pH 7.0], 250 mM NaCl, 5% (v/v) glycerol, 1 mM DTT). Cleaved protein was loaded onto a HiTrap SP column (GE Healthcare) and eluted over a linear KCl (0.25-1 M) gradient. LbuCas13a containing fractions were pooled, concentrated, and further purified via size-exclusion chromatography on a S200 column (GE Healthcare) in gel filtration buffer (20 mM HEPES [pH 7.0], 200 mM KCl, 5% glycerol (v/v), 1 mM DTT), snap-frozen in liquid N2 and were subsequently stored at −80° C.

### In-Vitro RNA Transcription

Mature crRNAs were synthetically made by IDT. All RNA targets were transcribed in vitro using previously described methods (Nature, 538, 270-273 (2016); RNA, 18, 661-672 (2012)). Briefly, all targets were transcribed off a single-stranded DNA oligonucleotide template (IDT) using T7 polymerase. were annealed to a 1.5-fold molar excess of an oligonucleotide corresponding to the T7 promoter sequence (5′-GGCGTAATACGACTCACTATAGG-3′ (SEQ ID NO:28)). Transcription reactions were incubated at 37° C. for 3 h and contained 1 μM template DNA, 100 μg/mL T7 polymerase, 1 μg/mL pyrophosphatase (Roche), 5 mM NTPs, 30 mM Tris-Cl (pHRT 8.1), 25 mM MgCl2, 10 mM dithiothreitol (DTT), 2 mM spermidine, and 0.01% Triton X-100. Reactions were then treated with 5 units of DNase (Promega) and incubated for an additional 30 min at 37° C. before being loaded on a 15% urea-polyacrylamide gel. Transcribed RNAs were purified using 15% Urea-PAGE. RNAs were excised from the gel and eluted into DEPC water overnight at 4° C. followed by ethanol precipitation. RNAs were resuspended in DEPC water and stored at −80° C. All sequences can be found in the following Table 2.

TABLE 2
Oligonucleotides used for in-vitro RNA transcription (SEQ ID
NOS: 28-63)

| No. | Name | Sequence |
|---|---|---|
| 28 | MOC-626_T7_opt | GGCGTAATACGACTCACTATAGG |
| 29 | MOC- | GTGTGGGCTTCTGCTGTGACAAATCTATCTGAATAAACTCTTC |
| | 1226_longer_AM_Liu_Lbu_ | TTCTTGGTTTCCCtatagtgagtcgtattacgcc |
| | target_IVTtemp | |
| 30 | MOC- | GTGTGGGCTTACTAAAACACAAATCTATCTGAATAAACTCTTC |
| | 1227_longer_AM_Liu_Lbu_ | TTCTTGGTTTCCCtatagtgagtegtattacgcc |
| | antitag_IVTtemp | |
| 31 | AM91_Lbu_Liu_target_ | GTGTGGGCTTCTGCTGTGG |
| | MM25:28 | ACAAATCTATCTGAATAAACTCTTGAAG |
| | | TTGGTTTCCCtatagtgagtcgtattacgcc |
| 32 | AM92_Lbu_Liu_target_ | GTGTGGGCTTCTGCTGTGG |
| | MM21:24 | ACAAATCTATCTGAATAAACAGAACTTC |
| | | TTGGTTTCCCtatagtgagtcgtattacgcc |
| 33 | AM93_Lbu_Liu_target_ | GTGTGGGCTTCTGCTGTGG |
| | MM17:20 | ACAAATCTATCTGAATTTTGTCTTCTTC |
| | | TTGGTTTCCCtatagtgagtcgtattacgcc |
| 34 | AM94_Lbu_Liu_target_ | GTGTGGGCTTCTGCTGTGG |
| | MM13:16 | ACAAATCTATCTCTTAAAACTCTTCTTC |
| | | TTGGTTTCCCtatagtgagtcgtattacgcc |
| 35 | AM95_Lbu_Liu_target_ | GTGTGGGCTTCTGCTGTGG |
| | MM9:12 | ACAAATCTTAGAGAATAAACTCTTCTTC |
| | | TTGGTTTCCCtatagtgagtcgtattacgcc |
| 36 | AM96_Lbu_Liu_target_ | GTGTGGGCTTCTGCTGTGG |
| | MM5:8 | ACAATAGAATCTGAATAAACTCTTCTTC |
| | | TTGGTTTCCCtatagtgagtcgtattacgcc |
| 37 | AM97_Lbu_Liu_target_ | GTGTGGGCTTCTGCTGTGG |
| | MM1:4 | TGTTATCTATCTGAATAAACTCTTCTTC |
| | | TTGGTTTCCCtatagtgagtegtattacgcc |
| 38 | AM108_Lbu_Liu_target_ | GTGTGGGCTTCTGCTGTGG |
| | SM1 | tCAAATCTATCTGAATAAACTCTTCTTC |
| | | TTGGTTTCCCtatagtgagtcgtattacgcc |
| 39 | AM109_Lbu_Liu_target_ | GTGTGGGCTTCTGCTGTGG |
| | SM2 | AgAAATCTATCTGAATAAACTCTTCTTC |
| | | TTGGTTTCCCtatagtgagtegtattacgcc |
| 40 | AM110_Lbu_Liu_target_ | GTGTGGGCTTCTGCTGTGG |
| | SM3 | ACtAATCTATCTGAATAAACTCTTCTTC |
| | | TTGGTTTCCCtatagtgagtcgtattacgcc |
| 41 | AM111_Lbu_Liu_target_ | GTGTGGGCTTCTGCTGTGG |
| | SM4 | ACAtATCTATCTGAATAAACTCTTCTTC |
| | | TTGGTTTCCCtatagtgagtcgtattacgcc |
| 42 | AM112_Lbu_Liu_target_ | GTGTGGGCTTCTGCTGTGG |
| | SM5 | ACAAtTCTATCTGAATAAACTCTTCTTC |
| | | TTGGTTTCCCtatagtgagtcgtattacgcc |
| 43 | AM113_Lbu_Liu_target_ | GTGTGGGCTTCTGCTGTGG |
| | SM6 | ACAAAaCTATCTGAATAAACTCTTCTTC |
| | | TTGGTTTCCCtatagtgagtcgtattacgcc |
| 44 | AM114_Lbu_Liu_target_S | GTGTGGGCTTCTGCTGTGG |
| | M7 | ACAAATgTATCTGAATAAACTCTTCTTC |
| | | TTGGTTTCCCtatagtgagtcgtattacgcc |
| 45 | AM115_Lbu_Liu_target_ | GTGTGGGCTTCTGCTGTGG |
| | SM8 | ACAAATCaATCTGAATAAACTCTTCTTC |
| | | TTGGTTTCCCtatagtgagtcgtattacgcc |
| 46 | AM116_Lbu_Liu_target_ | GTGTGGGCTTCTGCTGTGG |
| | SM9 | ACAAATCTTCTGAATAAACTCTTCTTC |
| | | TTGGTTTCCCtatagtgagtegtattacgcc |
| 47 | AM117_Lbu_Liu_target_ | GTGTGGGCTTCTGCTGTGG |
| | SM10 | ACAAATCTAaCTGAATAAACTCTTCTTC |
| | | TTGGTTTCCCtatagtgagtcgtattacgcc |
| 48 | AM118_Lbu_Liu_target_ | GTGTGGGCTTCTGCTGTGG |
| | SM11 | ACAAATCTATgTGAATAAACTCTTCTTC |
| | | TTGGTTTCCCtatagtgagtcgtattacgcc |
| 49 | AM119_Lbu_Liu_target_ | GTGTGGGCTTCTGCTGTGG |
| | SM12 | ACAAATCTATCaGAATAAACTCTTCTTC |
| | | TTGGTTTCCCtatagtgagtcgtattacgcc |
| 50 | AM120_Lbu_Liu_target_ | GTGTGGGCTTCTGCTGTGG |
| | SM13 | ACAAATCTATCTcAATAAACTCTTCTTC |
| | | TTGGTTTCCCtatagtgagtcgtattacgcc |
| 51 | AM121_Lbu_Liu_target_ | GTGTGGGCTTCTGCTGTGG |
| | SM14 | ACAAATCTATCTGtATAAACTCTTCTTC |
| | | TTGGTTTCCCtatagtgagtegtattacgcc |
| 52 | AM122_Lbu_Liu_target_ | GTGTGGGCTTCTGCTGTGG |
| | SM15 | ACAAATCTATCTGAtTAAACTCTTCTTC |
| | | TTGGTTTCCCtatagtgagtcgtattacgcc |
| 53 | AM123_Lbu_Liu_target_ | GTGTGGGCTTCTGCTGTGG |
| | SM16 | ACAAATCTATCTGAAaAAACTCTTCTTC |
| | | TTGGTTTCCCtatagtgagtcgtattacgcc |
| 54 | AM124_Lbu_Liu_target_ | GTGTGGGCTTCTGCTGTGG |
| | SM17 | ACAAATCTATCTGAATtAACTCTTCTTC |
| | | TTGGTTTCCCtatagtgagtcgtattacgcc |
| 55 | AM125_Lbu_Liu_target_ | GTGTGGGCTTCTGCTGTGG |
| | SM18 | ACAAATCTATCTGAATAtACTCTTCTTC |
| | | TTGGTTTCCCtatagtgagtcgtattacgcc |
| 56 | AM126_Lbu_Liu_target_ | GTGTGGGCTTCTGCTGTGG |
| | SM19 | ACAAATCTATCTGAATAAtCTCTTCTTC |
| | | TTGGTTTCCCtatagtgagtcgtattacgcc |
| 57 | AM127_Lbu_Liu_target_ | GTGTGGGCTTCTGCTGTGG |
| | SM20 | ACAAATCTATCTGAATAAAgTCTTCTTC |
| | | TTGGTTTCCCtatagtgagtegtattacgcc |
| 58 | AM160_SARS_COV2_S_ | CATTAAATGGTAGGACAGGGTTATCAAACCTCTTAGTACCATT |
| | D80_WT | GGTCCCAGAGACCCtatagtgagtcgtattacgcc |
| 59 | AM161_SARS_COV2_S_ | CATTAAATGGTAGGACAGGGTTAGCAAACCTCTTAGTACCATT |
| | D80_BETA | GGTCCCAGAGACCCtatagtgagtcgtattacgcc |
| 60 | AM164_SARS_COV2_S_ | TTAGACTTCCTAAACAATCTATACAGGTAATTATAATTACCAC |
| | L452_WT | CAACCTTAGAATCCtatagtgagtegtattacgcc |
| 61 | AM165_SARS_COV2_S_ | TTAGACTTCCTAAACAATCTATACCGGTAATTATAATTACCAC |
| | L452_DELTA | CAACCTTAGAATCCtatagtgagtcgtattacgcc |
| 62 | AM168_SARS_COV2_S_ | AAACCTTCAACACCATTACAAGGTGTGCTACCGGCCTGATAGA |
| | S477_WT | TTTCAGTTGAAACCtatagtgagtcgtattacgcc |
| 63 | AM169_SARS_COV2_S_ | AAACCTTCAACACCATTACAAGGTTTGTTACCGGCCTGATAGA |
| | S477_OMICRON | TTTCAGTTGAAACCtatagtgagtgtattacgcc |

Fluorescent ssRNA Nuclease Assays

Cas13 trans-cleavage nuclease activity assays were performed as previously described with some modifications (East-Seletsky et al., 2017). Briefly, 100 nM LbuCas13a: crRNA complexes were assembled in cleavage buffer (20 mM HEPES-Na pH 6.8, 50 mM KCl, 5 mM MgCl2, 10 μg/mL BSA, 100 μg/mL tRNA, 0.01% Igepal CA-630 and 5% glycerol), for 30 min 37° C. 100 nM of RNase Alert reporter (IDT) and various final concentrations of ssRNA-target were added to initiate the reaction. These reactions were incubated in a fluorescence plate reader (Tecan Spark) for up to 120 min at 37° C. with fluorescence measurements taken every 5 min (λex: 485 nm; λem: 535 nm). Time-course and end-point values at 1 hour were background-subtracted, normalized, and analyzed with their associated standard errors using Prism9 (GraphPad) and R version 4.1.1. Target RNAs and crRNAs used in the study can be found in Table 3 below.

TABLE 3
RNAs used in the ssRNA nuclease assays (SEQ ID NOS: 64-154)

| No. | Name | Sequence | Type |
|---|---|---|---|
| 64 | MOC- | GGACCACCCCAAAAAUGAAGGGGACUAAAAC | crRNA |
| | 1264_Lbu_mat_crRNA_Liu_28 nt | ACAAAUCUAUCUGAAUAAACUCUUCUUC | |
| 65 | MOC- | GGACCACCCCAAAAAUGAAGGGGACUAAAAC | crRNA |
| | 1265_Lbu_mat_crRNA_Liu_20 nt | ACAAAUCUAUCUGAAUAAAC | |
| 66 | MOC- | GGACCACCCCAAAAAUGAAGGGGACUAAAAC | crRNA |
| | 1266_Lbu_mat_crRNA_Liu_16 nt | ACAAAUCUAUCUGAAU | |
| 67 | AM208_Liu_mut_DR_28 | GGCCACCCCAAAAAUGAAGGGGACUAAAACA | crRNA |
| | | CAAAUCUAUCUGAAUAAACUCUUCUUC | |
| 68 | AM211_Liu_mut_DR_Lbu_20 | GGCCACCCCAAAAAUGAAGGGGACUAAAACA | crRNA |
| | | CAAAUCUAUCUGAAUAAAC | |
| 69 | MOC- | GGGAAACCAAGAAGAAGAGTTTATTCAGATAG | target |
| | 1284_Liu_long_target_IDT_RNA | ATTTGTCACAGCAGAAGCCCACAC | |
| 70 | MOC-1285_Liu_long_anti- | GGGAAACCAAGAAGAAGAGTTTATTCAGATAG | target |
| | target_IDT_RNA | ATTTGTGTTTTAGTAAGCCCACAC | |
| 71 | Liu_Perfect_match_target_RNA | GGGAAACCAA | target |
| | | GAAGAAGAGUUUAUUCAGAUAGAUUUGU | |
| | | CACAGCAGAAGCCCACAC | |
| 72 | Liu_target_RNA_MM1-4 | GGGAAACCAA | target |
| | | GAAGAAGAGUUUAUUCAGAUAGAUAACA | |
| | | CACAGCAGAAGCCCACAC | |
| 73 | Liu_target_RNA_MM5-8 | GGGAAACCAA | target |
| | | GAAGAAGAGUUUAUUCAGAUUCUAUUGU | |
| | | CACAGCAGAAGCCCACAC | |
| 74 | Liu_target_RNA_MM9-12 | GGGAAACCAA | target |
| | | GAAGAAGAGUUUAUUCUCUAAGAUUUGU | |
| | | CACAGCAGAAGCCCACAC | |
| 75 | Liu_target_RNA_MM13-16 | GGGAAACCAA | target |
| | | GAAGAAGAGUUUUAAGAGAUAGAUUUGU | |
| | | CACAGCAGAAGCCCACAC | |
| 76 | Liu_target_RNA_MM17-20 | GGGAAACCAA | target |
| | | GAAGAAGACAAAAUUCAGAUAGAUUUGU | |
| | | CACAGCAGAAGCCCACAC | |
| 77 | Liu_target_RNA_MM21-24 | GGGAAACCAA | target |
| | | GAAGUUCUGUUUAUUCAGAUAGAUUUGU | |
| | | CACAGCAGAAGCCCACAC | |
| 78 | Liu_target_RNA_MM25-28 | GGGAAACCAA | target |
| | | CUUCAAGAGUUUAUUCAGAUAGAUUUGU | |
| | | CACAGCAGAAGCCCACAC | |
| 79 | Liu_target_RNA_SM1 | GGGAAACCAA | target |
| | | GAAGAAGAGUUUAUUCAGAUAGAUUUGa | |
| | | CACAGCAGAAGCCCACAC | |
| 80 | Liu_target_RNA_SM2 | GGGAAACCAA | target |
| | | GAAGAAGAGUUUAUUCAGAUAGAUUUU | |
| | | CACAGCAGAAGCCCACAC | |
| 81 | Liu_target_RNA_SM3 | GGGAAACCAA | target |
| | | GAAGAAGAGUUUAUUCAGAUAGAUUaGU | |
| | | CACAGCAGAAGCCCACAC | |
| 82 | Liu_target_RNA_SM4 | GGGAAACCAA | target |
| | | GAAGAAGAGUUUAUUCAGAUAGAUaUGU | |
| | | CACAGCAGAAGCCCACAC | |
| 83 | Liu_target_RNA_SM5 | GGGAAACCAA | target |
| | | GAAGAAGAGUUUAUUCAGAUAGAaUUGU | |
| | | CACAGCAGAAGCCCACAC | |
| 84 | Liu_target_RNA_SM6 | GGGAAACCAA | target |
| | | GAAGAAGAGUUUAUUCAGAUAGUUUUGU | |
| | | CACAGCAGAAGCCCACAC | |
| 85 | Liu_target_RNA_SM7 | GGGAAACCAA | target |
| | | GAAGAAGAGUUUAUUCAGAUAcAUUUGU | |
| | | CACAGCAGAAGCCCACAC | |
| 86 | Liu_target_RNA_SM8 | GGGAAACCAA | target |
| | | GAAGAAGAGUUUAUUCAGAUUGAUUUGU | |
| | | CACAGCAGAAGCCCACAC | |
| 87 | Liu_target_RNA_SM9 | GGGAAACCAA | target |
| | | GAAGAAGAGUUUAUUCAGAaAGAUUUGU | |
| | | CACAGCAGAAGCCCACAC | |
| 88 | Liu_target_RNA_SM10 | GGGAAACCAA | target |
| | | GAAGAAGAGUUUAUUCAGUUAGAUUUGU | |
| | | CACAGCAGAAGCCCACAC | |
| 89 | Liu_target_RNA_SM11 | GGGAAACCAA | target |
| | | GAAGAAGAGUUUAUUCACAUAGAUUUGU | |
| | | CACAGCAGAAGCCCACAC | |
| 90 | Liu_target_RNA_SM12 | GGGAAACCAA | target |
| | | GAAGAAGAGUUUAUUCUGAUAGAUUUGU | |
| | | CACAGCAGAAGCCCACAC | |
| 91 | Liu_target_RNA_SM13 | GGGAAACCAA | target |
| | | GAAGAAGAGUUUAUUgAGAUAGAUUUGU | |
| | | CACAGCAGAAGCCCACAC | |
| 92 | Liu_target_RNA_SM14 | GGGAAACCAA | target |
| | | GAAGAAGAGUUUAUaCAGAUAGAUUUGU | |
| | | CACAGCAGAAGCCCACAC | |
| 93 | Liu_target_RNA_SM15 | GGGAAACCAA | target |
| | | GAAGAAGAGUUUAaUCAGAUAGAUUUGU | |
| | | CACAGCAGAAGCCCACAC | |
| 94 | Liu_target_RNA_SM16 | GGGAAACCAA | target |
| | | GAAGAAGAGUUUUUUCAGAUAGAUUUGU | |
| | | CACAGCAGAAGCCCACAC | |
| 95 | Liu_target_RNA_SM17 | GGGAAACCAA | target |
| | | GAAGAAGAGUUaAUUCAGAUAGAUUUGU | |
| | | CACAGCAGAAGCCCACAC | |
| 96 | Liu_target_RNA_SM18 | GGGAAACCAA | target |
| | | GAAGAAGAGUaUAUUCAGAUAGAUUUGU | |
| | | CACAGCAGAAGCCCACAC | |
| 97 | Liu_target_RNA_SM19 | GGGAAACCAA | target |
| | | GAAGAAGAGaUUAUUCAGAUAGAUUUGU | |
| | | CACAGCAGAAGCCCACAC | |
| 98 | Liu_target_RNA_SM20 | GGGAAACCAA | target |
| | | GAAGAAGAcUUUAUUCAGAUAGAUUUGU | |
| | | CACAGCAGAAGCCCACAC | |
| 99 | AM160_SARS_COV2_S_D80_WT | GGGTCTCTGGGACCAATGGTACTAAGAGGTTT [symbol] | target |
| | | ATAACCCTGTCCTACCATTTAATG | |
| 100 | AM161_SARS_COV2_S_D80_ | GGGTCTCTGGGACCAATGGTACTAAGAGGTTT [symbol] | target |
| | BETA | CTAACCCTGTCCTACCATTTAATG | |
| 101 | AM164_SARS_COV2_S_L452_ | GGATTCTAAGGTTGGTGGTAATTATAATTACCT | target |
| | WT | GTATAGATTGTTTAGGAAGTCTAA | |
| 102 | AM165_SARS_COV2_S_L452_ | GGATTCTAAGGTTGGTGGTAATTATAATTACCG | target |
| | DELTA | GTATAGATTGTTTAGGAAGTCTAA | |
| 103 | AM168_SARS_COV2_S_S477_ | GGTTTCAACTGAAATCTATCAGGCCGGTAGCA [symbol] | target |
| | WT | ACCTTGTAATGGTGTTGAAGGTTT | |
| 104 | AM169_SARS_COV2_S_S477_ | GGTTTCAACTGAAATCTATCAGGCCGGTAACAA | target |
| | OMICRON | ACCTTGTAATGGTGTTGAAGGTTT | |
| 105 | AM162_WT_crRNA_S_D80_nt | GGACCACCCCAAAAAUGAAGGGGACUAAAAC | target |
| | | GGGUUAUCAAACCUCUUAGU | |
| 106 | AM163_beta_crRNA_S_D80_20 nt | GGACCACCCCAAAAAUGAAGGGGACUAAAAC | crRNA |
| | | GGGUUAGCAAACCUCUUAGU | |
| 107 | AM166_WT_crRNA_L452_S_20 nt | GGACCACCCCAAAAAUGAAGGGGACUAAAAC | crRNA |
| | | CUAUACAGGUAAUUAUAAUU | |
| 108 | AM167_Delta_crRNA_L452R_S_ | GGACCACCCCAAAAAUGAAGGGGACUAAAAC | crRNA |
| | 20 nt | CUAUACCGGUAAUUAUAAUU | |
| 109 | AM170_WT_crRNA_S477_S_20 nt | GGACCACCCCAAAAAUGAAGGGGACUAAAAC | crRNA |
| | | CAAGGUGUGCUACCGGCCUG | |
| 110 | AM171_omicron_crRNA_477_ | GGACCACCCCAAAAAUGAAGGGGACUAAAAC | crRNA |
| | 478S_20 nt | CAAGGUUUGUUACCGGCCUG | |
| 111 | AM194_WT_crRNA_S_D80_20 nt_ | GGACCACCCCAAAAAUGAAGGGGACUAAAAC | crRNA |
| | MM19 | GGGUUAUCAAACCUCUUACU | |
| 112 | AM195_beta_crRNA_S_D80_20 nt_ | GGACCACCCCAAAAAUGAAGGGGACUAAAAC | crRNA |
| | MM7_19 | GGGUUAGCAAACCUCUUACU | |
| 113 | AM196_WT_crRNA_L452_S_20 nt_ | GGACCACCCCAAAAAUGAAGGGGACUAAAAC | crRNA |
| | MM19 | CUAUACAGGUAAUUAUAAAU | |
| 114 | AM197_Delta_crRNA_L452R_S_ | GGACCACCCCAAAAAUGAAGGGGACUAAAAC | crRNA |
| | 20 nt_MM7_19 | CUAUACCGGUAAUUAUAAAU | |
| 115 | AM198_WT_crRNA_S477_S_20 nt_ | GGACCACCCCAAAAAUGAAGGGGACUAAAAC | crRNA |
| | MM19 | CAAGGUGUGCUACCGGCCAG | |
| 116 | AM199_omicron_crRNA_477_478_ | GGACCACCCCAAAAAUGAAGGGGACUAAAAC | crRNA |
| | S_20 nt_MM7_19 | CAAGGUUUGUUACCGGCCAG | |
| 117 | AM202_wt_crRNA_D80_v2 | GGACCACCCCAAAAAUGAAGGGGACUAAAAC | crRNA |
| | | AAUGGUAGGACAGGGUUAUC | |
| 118 | AM203_beta_crRNA_D80_v2_ | GGACCACCCCAAAAAUGAAGGGGACUAAAAC | crRNA |
| | MM19 | AAUGGUAGGACAGGGUUAGC | |
| 118 | AM204_wt_crRNA_L452_v2 | GGACCACCCCAAAAAUGAAGGGGACUAAAAC | crRNA |
| | | UUCCUAAACAAUCUAUACAG | |
| 120 | AM205_delta_crRNA_L452_v2_ | GGACCACCCCAAAAAUGAAGGGGACUAAAAC | crRNA |
| | MM19 | UUCCUAAACAAUCUAUACCG | |
| 121 | AM206_wt_crRNA_S477_v2 | GGACCACCCCAAAAAUGAAGGGGACUAAAAC | crRNA |
| | | ACACCAUUACAAGGUGUGCU | |
| 122 | AM207_omicron_crRNA_S477_v2_ | GGACCACCCCAAAAAUGAAGGGGACUAAAAC | crRNA |
| | MM19 | ACACCAUUACAAGGUUUGUU | |
| 123 | AM212_WT_del_crRNA_S_D80_ | GGCCACCCCAAAAAUGAAGGGGACUAAAACG | crRNA |
| | nt | GGUUAUCAAACCUCUUAGU | |
| 124 | AM213_beta_del_crRNA_S_D80_ | GGCCACCCCAAAAAUGAAGGGGACUAAAACG | crRNA |
| | 20 nt | GGUUAGCAAACCUCUUAGU | |
| 125 | AM214_WT_del_crRNA_L452_S_ | GGCCACCCCAAAAAUGAAGGGGACUAAAACC | crRNA |
| | 20 nt | UAUACAGGUAAUUAUAAUU | |
| 126 | AM215_Delta_del_crRNA_L452R_ | GGCCACCCCAAAAAUGAAGGGGACUAAAACC | crRNA |
| | S_20 nt | UAUACCGGUAAUUAUAAUU | |
| 127 | AM216_WT_del_crRNA_S477_S | GGCCACCCCAAAAAUGAAGGGGACUAAAACC | crRNA |
| | 20nt | AAGGUGUGCUACCGGCCUG | |
| 128 | AM217_omicron_del_crRNA_477_ | GGCCACCCCAAAAAUGAAGGGGACUAAAACC | crRNA |
| | 478_S_20 nt | AAGGUUUGUUACCGGCCUG | |
| 129 | AM222_crRNA_S_D80_20 nt_ | GGCCACCCCAAAAAUGAAGGGGACUAAAACG | crRNA |
| | MM19_mut_DR | GGUUAUCAAACCUCUUACU | |
| 130 | AM223_beta_crRNA_S_D80_20 nt_ | GGCCACCCCAAAAAUGAAGGGGACUAAAACG | crRNA |
| | MM7_19_mut_DR | GGUUAGCAAACCUCUUACU | |
| 131 | AM224_WT_crRNA_L452_S_20 nt_ | GGCCACCCCAAAAAUGAAGGGGACUAAAACC | crRNA |
| | MM19_mut_DR | UAUACAGGUAAUUAUAAAU | |
| 132 | AM225_Delta_crRNA_L452R_S_ | GGCCACCCCAAAAAUGAAGGGGACUAAAACC | crRNA |
| | 20 nt_MM7_19_mut_DR | UAUACCGGUAAUUAUAAAU | |
| 133 | AM226_WT_crRNA_S477_S_20 nt_ | GGCCACCCCAAAAAUGAAGGGGACUAAAACC | crRNA |
| | MM19_mut_DR | AAGGUGUGCUACCGGCCAG | |
| 134 | AM227_omicron_crRNA_477_478_ | GGCCACCCCAAAAAUGAAGGGGACUAAAACC | crRNA |
| | S_20 nt_MM7_19_mut_DR | AAGGUUUGUUACCGGCCAG | |
| 135 | AM228_Liu_mut_DR_Lbu_20_ | GGCCACCCCAAAAAUGAAGGGGACUAAAACA | crRNA |
| | pos7_G | CAAAUGUAUCUGAAUAAAC | |
| 136 | AM229_Liu_mut_DR_Lbu_20_ | GGCCACCCCAAAAAUGAAGGGGACUAAAACA | crRNA |
| | pos7_A | CAAAUAUAUCUGAAUAAAC | |
| 137 | AM230_Liu_mut_DR_Lbu_20_ | GGCCACCCCAAAAAUGAAGGGGACUAAAACA | crRNA |
| | pos7_U | CAAAUUUAUCUGAAUAAAC | |
| 138 | AM243_WT_del_crRNA_S_D80_ | GGCCACCCCAAAAAUGAAGGGGACUAAAACG | crRNA |
| | 20 nt_MM6 | GGUUUUCAAACCUCUUAGU | |
| 139 | AM244_beta_del_crRNA_S_D80_ | GGCCACCCCAAAAAUGAAGGGGACUAAAACG | crRNA |
| | 20 nt_MM6 | GGUUUGCAAACCUCUUAGU | |
| 140 | AM245_WT_del_crRNA_L452_S_ | GGCCACCCCAAAAAUGAAGGGGACUAAAACC | crRNA |
| | 20 nt_MM6 | UAUAGAGGUAAUUAUAAUU | |
| 141 | AM246_Delta_del_crRNA_L452R_ | GGCCACCCCAAAAAUGAAGGGGACUAAAACC | crRNA |
| | S_20 nt_MM6 | UAUAGCGGUAAUUAUAAUU | |
| 142 | AM247_WT_del_crRNA_S477_S_ | GGCCACCCCAAAAAUGAAGGGGACUAAAACC | crRNA |
| | 20 nt_MM6 | AAGGAGUGCUACCGGCCUG | |
| 143 | AM248_omicron_del_crRNA_477_ | GGCCACCCCAAAAAUGAAGGGGACUAAAACC | crRNA |
| | 478_S_20 nt_MM6 | AAGGAUUGUUACCGGCCUG | |
| 144 | AM249_WT_crRNA_S_D80_20 nt | GGACCACCCCAAAAAUGAAGGGGACUAAAAC | crRNA |
| | MM6_19 | GGGUUUUCAAACCUCUUACU | |
| 145 | Liu_modified_localGC_3_G | GGGAAACCAA | target |
| | | GAAUAAGAGCUCACUCGGAUACAUUUGC | |
| | | CACAGCAGAAGCCCACAC | |
| 146 | Liu_modified_localGC_3_C_MM | GGGAAACCAA | target |
| | | GAAUAAGAGCUCACUCGGAUAgAUUUGC | |
| | | CACAGCAGAAGCCCACAC | |
| 147 | Liu_modified_totalGC_50_G | GGGAAACCAA | target |
| | | GAAGAAGAAUUUAUUUAGAUGCGCUUAU | |
| | | CACAGCAGAAGCCCACAC | |
| 148 | Liu_modified_totalGC_50_C_MM | GGGAAACCAA | target |
| | | GAAGAAGAAUUUAUUUAGAUGgGCUUAU | |
| | | CACAGCAGAAGCCCACAC | |
| 149 | AM254_Lbu_mut_DR_modified_ | GGCCACCCCAAAAAUGAAGGGGACUAAAACA | crRNA |
| | localGC_3_C | UAAGCCCAUCUAAAUAAAU | |
| 150 | AM255_Lbu_mut_DR_modified_ | GGCCACCCCAAAAAUGAAGGGGACUAAAACA | crRNA |
| | localGC_3_G | UAAGCGCAUCUAAAUAAAU | |
| 151 | AM256_Lbu_mut_DR_modified_ | GGCCACCCCAAAAAUGAAGGGGACUAAAACG | crRNA |
| | totalGC_50_C | CAAAUCUAUCCGAGUGAGC | |
| 152 | AM257_Lbu_mut_DR_modified_ | GGCCACCCCAAAAAUGAAGGGGACUAAAACG | crRNA |
| | totalGC_50_G | CAAAUGUAUCCGAGUGAGC | |
| 153 | AM261_WT_del_crRNA_L452R_ | GGCCACCCCAAAAAUGAAGGGGACUAAAACU | crRNA |
| | S_20 nt_MM19 | UCCUAAACAAUCUAUACAG | |
| 154 | AM262_Delta_del_crRNA_L452R_ | GGCCACCCCAAAAAUGAAGGGGACUAAAACU | crRNA |
| | S_20 nt_MM19 | UCCUAAACAAUCUAUACCG | |

[symbol]

indicates data missing or illegible when filed

For lateral flow based detection, we generated the reaction mix as described above, except the study used a biotinylated FAM reporter at a final concentration of 1 μM rather than the RNAse Alert substrate. After 30 minutes of incubation at 37° C., the detection reaction was diluted 1:4 in Milenia HybriDetect Assay Buffer, and the Milenia HybriDetect 1 (TwistDx) lateral flow strip was added. Sample images were collected 5 min following incubation of the strip.

### Structural Models

Molecular simulations were based on the structure of the Leptotrichia buccalis (Lbu) Cas13a bound to a crRNA (PDB: 5XWY, at 3.2 Å resolution (Cell, 2017, 168, 121-134.e12)), obtained via cryo-EM, and on the structure of the LbuCas13a in complex with a crRNA and tgRNA (PDB: 5XWP), obtained by single-wavelength anomalous diffraction at 3.08 Å resolution. The LbuCas13a bound to an extended tag-anti-tag RNA (atgRNA) was built by including a longer duplex with eight base-pair extended atgRNA, obtained from the cryo-EM structure of the Leptotrichia shahii Cas13a bound to atgRNA (PDB: 7DMQ, at 3.06 Å resolution (Cell, 2021, 81, 1100-1115)). Additionally, we have also considered the four variants (R377A, N378A, R963A, and R973A) of Cas13a bound to a tgRNA, including an atgRNA, similar to the wild-type (WT) Cas13a complexes. To study the impact of mismatches, we have also modeled four Cas13a systems with guide: target complementarity length of 20 nucleotides: crRNA: including either a perfectly matched crRNA: target-RNA duplex, or crRNA: target-RNA duplexes that contain a single mismatch at either spacer nucleotide position 4, 7, or 11. These systems are made of guide RNA sequence length of 20 nt. The sequences of spacer crRNA and target RNAs (tgRNA) that are used for the complexes are the same as those used for their corresponding cleavage assays. In all systems, we reinstated the catalytic H1053 and R1048 in the HEPN domains, which were mutated in alanine in the experimental structures (see Cell, 2017, 168, 121-134.e12). The protonation states of histidine residues have been computed using the H++ software (NAR.2005, 33, W368-W371) reporting singly protonated neutral states (protonated on the ε position). This allows histidine residues to act as a base within the HEPN1-2 cleft, as recently shown for a type III CRISPR-Cas system holding the HEPN1-2 RNase activity (see Garcia-Doval et al. Nat. Commun. 2022, 11, 1596). All systems were solvated and neutralized by the addition of an adequate number of Na+ ions.

### Molecular Dynamics Simulations

MD simulations were performed by employing a simulation protocol tailored for protein/nucleic acid complexes. The study employed the Amber ff19SB force field (J. Chem. Theory Comput., 2020, 16, 528-552) with the χOL3 corrections for RNA (see J. Chem. Theory Comput., 2010, 6, 3836-3849; J. Chem. Theory Comput., 2011, 7, 2886-29020). The TIP3P model was used for explicit water molecules (The Journal of Chemical Physics, 1983, 79, 926-935). As a first step, all systems were subjected to energy minimization to overcome the potential inter- and intra-molecular steric clashes. Then, the systems were heated from 0 to 100 K in two consecutive NVT simulations (representing the canonical ensemble) of 5 ps each, imposing positional restraints of 100 kcal/mol Å2 on the protein-RNA complex. The temperature was further increased up to 200 K in a subsequent ˜100 ps MD run in the isothermal-isobaric ensemble (NPT), in which the restraint was reduced to 25 kcal/mol Å². Finally, all restraints were released, and the systems were heated up to 300 K in a single NPT simulation of 500 ps. These simulations were performed using a 1 fs time step. The simulation time step was subsequently increased to 2 fs for further equilibration and production simulations. All bond lengths involving hydrogen atoms were constrained using the SHAKE algorithm (Journal of Computational Physics, 1977, 23 (3): 327-341). After ˜600 ps of equilibration, ˜10 ns of NPT simulation were carried out, allowing the systems' density to stabilize around 1.01 g/cm⁻³. The temperature was kept constant at 300 K via Langevin dynamics (J. Chem. Phys., 1977, 66, 3039), with a collision frequency γ=1 ps⁻¹. The pressure was controlled in the NPT simulations by coupling the system to a Berendsen barostat (The Journal of Chemical Physics, 1984, 81, 3684-3690) at a reference pressure of 1 atm and with a relaxation time of 2 ps. Finally, production runs were carried out for all RNA-bound WT systems (i.e., crRNA-Cas13a, tgRNA-Cas13a, and atgRNA-Cas13a) in the NVT ensemble, reaching ˜5 μs and in three replicates, accumulating ˜45 μs of total sampling.

Following the same strategy, the study also performed a ˜5 μs long simulation of a target RNA-bound complex substituting the A(−3) base of the crRNA with cytosine C(−3). Subsequently, the study also considered four mutated variants (R377A, N378A, R963A, and R973A) of LbuCas13a bound to a tgRNA, and including a tag-anti-tag RNA (i.e., atgRNA bound), similar and starting from the wild-type (WT) LbuCas13a complexes. These systems were simulated in three replicates of ˜5 μs each, totaling ˜120 μs of accumulated sampling for the variant-bound complexes. The 20 nucleotide guide: target duplex containing systems were also simulated in three replicates, reaching ˜1 μs for each replica, accumulating ˜12 μs of total sampling. Overall, here the study reports the results from ˜180 μs of accumulated sampling. All simulations were performed using the GPU-empowered version of the AMBER 20 simulation package (Case et al., (2020) AMBER 2020. University of California, San Francisco). Analyses were performed over the aggregated multi-μs sampling collected for each of the studied complexes, offering a robust solid ensemble for the purposes of the analysis. Enhanced sampling simulations were also performed to compute the free energy profiles associated with the flipping of single nucleotide mismatches in the tgRNA.

### Dynamic Network Analysis and Signal-to-Noise Ratio

To characterize the allosteric pathways of communication, network analysis was applied (Proc. Natl. Acad. Sci. U.S.A., 2009, 106, 6620-6625). In dynamical networks, Ca atoms of proteins and backbone P atoms of nucleotides, as well as N1 atoms in purines, and N9 in pyrimidines, are represented as nodes. To determine whether nodes are involved in effective contacts, two criteria are used: (i) a distance cut-off of 4.5 Å between any two heavy atoms of two residues, and (ii) a frequency cut-off of 0.75, such that contacts are considered if formed for at least the 75% of the simulation time. Nodes are connected by edges weighted by the generalized correlations GCᵢⱼ according to:

$${W}_{ij} = - log ( {GC}_{ij} ) \qquad ( 1 )$$

From the dynamical network, the study estimated the efficiency of crosstalk between the crRNA spacer regions (i.e., “seed” (nt. 9-14); “switch” (nt. 5-8)) and the catalytic residues (R472, H477, R1048, H1053) by introducing a novel Signal-to-Noise Ratio (SNR) measure. SNR measures the preference of communication between predefined distant sites—i.e., the signal—over the remaining pathways in the network—i.e., the noise, estimating how allosteric pathways stand out (i.e., are favorable) over the entire communication network. The study computed the pathways for all source-sink pairs (i.e., considering all source nucleotides and all sink residues), leading to a total number of pathways, Nₚₐₜₕ:

$${N}_{path} = {N}_{path - source - sink} \times {N}_{source - nt} \times {N}_{sink - res} \qquad ( 2 )$$

where N_{path-source-sink} is the number of optimal and suboptimal pathways for each source-sink pair, N_{source-nt} is the number of source nucleotides, while Nₛᵢₙₖ₋ᵣₑₛ is the number of catalytic residues. The study then considered Nₚₐₜₕ for the calculation of the SNR and occurrence of residues in the “seed”/“switch”—catalytic core communication.

For the SNR calculation, the study first computed the optimal (i.e., the shortest) and top five sub-optimal pathways (with longer lengths, ranked compared to the optimal path length) between all crRNA bases and the Cas13a residues, using well-established algorithms (vide infra). Then, the cumulative betweennesses of each pathway (Sₖ) was calculated as the sum of the betweennesses of all the edges in that specific pathway:

$${S}_{k} = {\sum }_{i = 1}^{n - 1} {b}_{i} \qquad ( 3 )$$

where bᵢ is the edge betweenness (i.e., the number of shortest pathways that cross the edge, measuring the “traffic” passing through them) between node i and i+1, and n is the number of edges in the kth pathway. The distribution of Sₖ between the crRNA bases and all protein residues was defined as the noise, whereas the distribution of Sₖ between the crRNA nucleotide regions of interest (e.g., “seed”) and the HEPN1-2 catalytic residues were considered as signals. Notably, while the optimal path corresponds to the most likely mode of communication, suboptimal paths can also be crucial routes for communication transfer (see J. Chem. Phys., 2020, 153, 134104; J Am Chem Soc, 2020. 142 (3): p. 1348-1358). Hence, in addition to the optimal path, the study also considered the top five sub-optimal pathways for the SNR analysis. Well-established algorithms were employed for shortest-path analysis. The Floyd-Warshall algorithm was utilized to compute the optimal paths between the network nodes. The five sub-optimal paths were computed in rank from the shortest to the longest, using Yen's algorithm, which computes single-source K-shortest loop-less paths (i.e., without repeated nodes) for a graph with non-negative edge weights (see Manage. Sci., 1971, 17, 712-716). To identify residues important for allosteric communication, the study computed the occurrence of each residue appearing in at least one of the pathways (i.e., optimal and sub-optimal). This analysis also reports on the conservation of allosteric pathways, as pathways characterized by a lesser number of residues with higher occurrence are likely to be more conserved than those exhibiting a greater number of residues with lower occurrence.

Finally, the corresponds to signals from each crRNA region (i.e., “seed” (nt. 9-14); “switch” (nt. 5-8) to the HEPN1-2 catalytic core residues (R472, H477, R1048, H1053) was computed as:

$$S N R = \frac{E [ S ] / Var ( S )}{E [ N ] / Var ( N )} \qquad ( 4 )$$

where E(S) and Var(S) correspond to the expectation and variance of the signal distribution respectively; and E(N)/Var(N) is the expectation/variance of the noise distribution. All networks were built using the Dynetan Python library (see J. Chem. Phys., 2020, 153, 134104). Path-based analyses were performed using NetworkX Python library (see Hagberg, A., Schult, D. and Swart, P. Exploring Network Structure, Dynamics, and Function using NetworkX in Proceedings of the 7th Python in Science conference (SciPy 2008). G Varoquaux, T Vaught, J Millman (Eds.), 11-15) and the in-house Python scripts.

### SARS-COV-2 Propagation

The following reagents were deposited by the Centers for Disease Control and Prevention and obtained through BEI Resources, NIAID, NIH: SARS-Related Coronavirus 2, isolate Hong Kong/VM20001061/2020, NR-52282; Isolate South Africa/KRISP-EC-K005321/2020 (B.1.351 lineage), NR-54008; isolate South Africa/KRISP-K005325/2020 (B.1.351 lineage), NR-54009; isolate USA/PHC658/2021 (Lineage B.1.617.2; Delta variant, NR-55611; and isolate USA/MD-HP20874/2021 (Lineage B.1.1.529; Omicron variant), NR-56461. SARS-COV-2 was propagated (MOI of 0.1) and titered using 80% confluent African green monkey kidney epithelial Vero E6 cells (American Type Culture Collection, CRL-1586) or Vero-hACE2-TMPRSS2 cells (Vero AT) (BEI NR-54970) in Eagle's Minimum Essential Medium (Lonza, 12-125Q) supplemented with 2% fetal bovine serum (FBS) (Atlanta Biologicals), 2 mM 1-glutamine (Lonza, BE17-605E), and 1% penicillin (100 U/ml) and streptomycin (100 μg/ml) or puromycin (10 ug/ml) (Thermo Fisher, A11138-03). All isolates except Omicron were propagated and titered in Vero EG cells and using penicillin and streptomycin. The Omicron variant was propagated and titered in Vero AT cells using puromycin. Virus stock was stored at −80° C. All work involving infectious SARS-COV-2 was performed in the Biosafety Level 3 (BSL-3) core facility of the University of Rochester, with institutional biosafety committee (IBC) oversight.

### Tissue Culture Infectious Dose Assay, Viral Inactivation, and RNA Extraction

Viral titers were determined using the tissue culture infectious dose (TCID) assay on triplicate wells of an 80% confluent monolayer of Vero E6 cells in a 96-well microtiter plate format using a 1:3 dilution factor; virus infection was assessed following 3-5 days of incubation at 37° C. in a CO2 incubator by microscopic examination of cytopathic effects (CPE). The infectious dose (log 10 TCID50/ml) was calculated using the Spearman-Kärber method (65,66). TCID50 of approximately seven logarithms per milliliter were used for RNA extractions. Infectious viral stocks were inactivated by a 1:3 dilution with TRI Reagent® (Zymo, R2050-1-200) immediately prior to RNA extraction. RNA was extracted using the Direct-zol RNA Miniprep Plus (Zymo, R2073) according to the manufacturer's protocol, including on-column DNase 1 treatment.

### Viral and Extracted Sample Preparation and RT-qPCR Testing

To assess quality and relative quantity of viral RNA, RT-qPCR was performed using the Luna® SARS-COV-2 RT-qPCR Multiplex Assay Kit (NEB) with the CDC-derived primers for N1 and N2 gene targets and the reaction was performed using the QuantStudio™ 5 System (ThermoFisher).

For Cas13-cleavage assays, viral RNA was reversed transcribed using the High-Capacity cDNA Reverse Transcription Kit (Applied Biosystems). cDNA was amplified for the regions of interest using the primers listed in Table 4 below.

TABLE 4
Oligonucleotides used for SARS-CoV-2 amplification (SEQ ID
NOS: 155-160)

| No. | Name | Sequence 5′-3′ |
|---|---|---|
| 155 | AM238_SARS_CoV2_D80A_fwd_ | GAAATTAATACGACTCACTATAGGG |
| | Arizti-Sanz | CAACTCAGGACTTGTTCTTACCTTTCTTTT |
| | | CC |
| 156 | AM239_SARS_CoV2_D80A_ | AAGCAAAATAAACACCATCATTAAAT |
| | rev_Arizti-Sanz | |
| 157 | AM263_RPA_fwd_Yang_SARS_ | GAAATTAATACGACTCACTATAGGG |
| | S452_T7 | CTTGATTCTAAGGTTGGTGGTAATTATAAT |
| 158 | AM264_RPA_rev_Yang_SARS_ | AAGGTTTGAGATTAGACTTCCTAAACAAT |
| | S452 | C |
| 159 | AM220_SARS_CoV2_T478K_fwd_ | GAAATTAATACGACTCACTATAGGG |
| | Yang | TTGAGAGAGATATTTCAACTGAAATCTAT |
| | | C |
| 160 | AM221 | AGTGGGTTGGAAACCATATGATTGTAAAG |
| | SARS_CoV2_T478K_rev_Yang | G |

The forward primers introduced a T7 RNAP promoter. PCR amplification was carried out using Q5® High-Fidelity DNA Polymerase (NEB) for at least 35 cycles, and annealing temperature of 60° C. Reaction products were visualized on a 2% agarose gel with 0.05% (v/v) Ethidium Bromide and visualized with a Gel Doc XR+ imager (Bio-Rad Laboratories). PCR amplicons were column purified with the Monarch® PCR & DNA Cleanup Kit and eluted in 25 μL of Monarch® DNA Elution Buffer.

For the detection step, 1 μL of purified amplification product was added to 19 μL detection master mix (100 nM LbuCas13a: crRNA in cleavage buffer (20 mM HEPES-Na pH 6.8, 50 mM KCl, 5 mM MgCl2, 10 μg/mL BSA, 100 μg/mL tRNA, 0.01% Igepal CA-630 and 5% glycerol) with 1 U/μL murine RNase inhibitor (NEB), 0.1 μg/μL T7 RNA polymerase (purified in-house) and 1 mM of rNTP mix.

### Example 2: Computational Design of High Fidelity Cas13a Variants

To understand the biophysical mechanisms underlying the function and specificity of Cas13a, and to gain insights at the molecular level for biomolecular engineering, the study performed extensive molecular dynamics simulations and analyzed through graph theory. Starting from the cryo-EM and X-ray structures of Cas13a bound to a crRNA (PDB: 5XWP) and a target RNA (PDB: 5XWY and 7DMQ), the study performed all-atom molecular dynamics (MD) simulations of the inactive Cas 13a protein bound to a guide crRNA (viz., crRNA-Cas 13a; FIG. 1, Panel A) and two ternary complexes bound to a target RNA. In the first ternary complex (viz., tgRNA-Cas 13a), the target RNA binds to the crRNA with 28 nt complementarity (FIG. 1, Panel B). The second ternary complex (viz., atgRNA-Cas 13a, FIG. 1, Panel C) displays an extended complementarity of 36 base pairs between the crRNA and the target RNA. Such extended pairing is called tag (segment from the crRNA)-antitag (segment from the tgRNA) pairing. Recent experimental studies have reported that extending the target-guide complementarity has severe impact on the RNA degrading ability of Cas 13a. This indicated for a competitive allosteric regulation, dependent on the status of complementarity between guide the crRNA and the tgRNA. Hence, comparing dynamics and mechanism of the binary and the ternary complexes of Cas 13a at the atomic level should be insightful to understand the nuclease activation and to perform rational design of improved function. The study therefore performed extensive molecular simulations of the complexes, collecting an aggregate sampling of ˜15 μs for each system, resulting in a total ensemble of ˜45 μs.

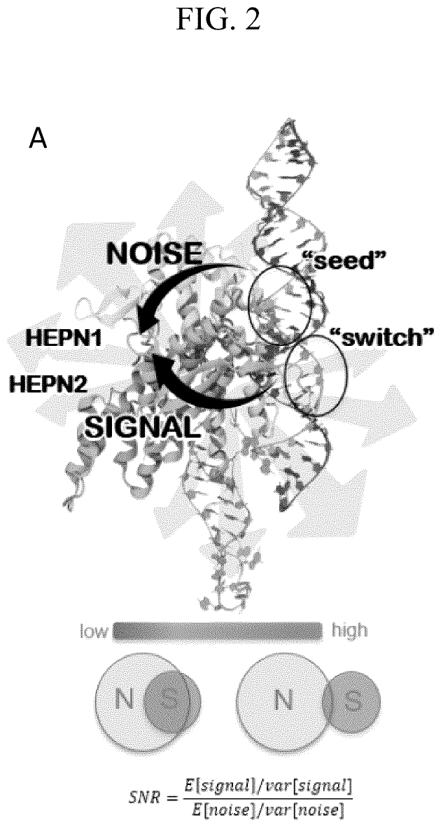

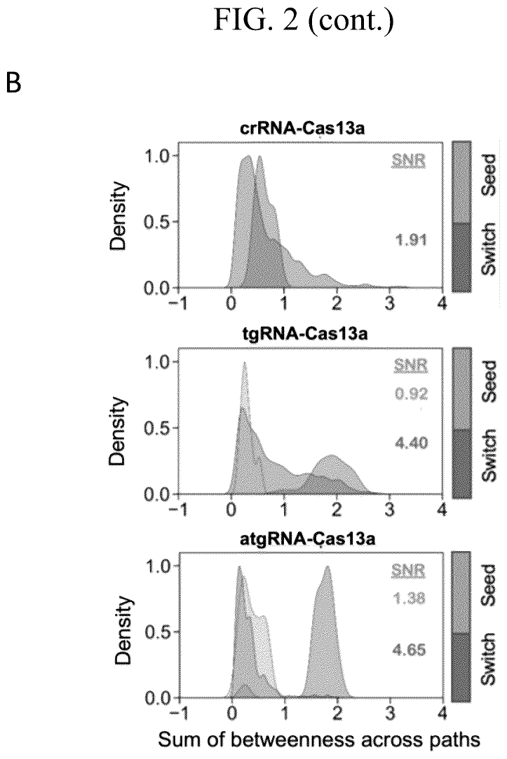

The target RNA binds Cas13a by matching the crRNA, while target RNA cleavage occurs at distal sites, within the HEPN1-2 catalytic cleft (FIG. 1). To understand the mechanism of information transfer and how dynamical differences arising from target RNA binding impact allosteric signaling, the study estimated the communication efficiency between the crRNA spacer regions (i.e., the sites of target RNA binding) and the catalytic cleft using graph theory-based network analysis. This has been shown to efficiently describe allosteric effects and to guide the engineering of improved CRISPR-Cas9 genome editing tools (see J Am Chem Soc, 2020. 142 (3): p. 1348-1358; J Am Chem Soc, 2017. 139 (45): p. 16028-16031). Applying this approach, the study represented the CRISPR-Cas13a system as a network of residue nodes and edges. The study then calculated the shortest pathways for information transfer between the target RNA binding sites and the catalytic residues (R472, H477, R1048, H1053) (FIG. 2, Panel A). The study then introduced a Signal-to-Noise Ratio (SNR) of communication efficiency to estimate the preference of the communication between predefined distant sites—i.e., the signal—over the remaining pathways in the network—i.e., the noise (see Materials and Methods). High SNR values indicate a preference of the network to communicate through the signal (i.e., communication between a specific source-sink pair) over other noisy routes (i.e., communications between all other node/residue pairs) (FIG. 2, Panel B).

For path analysis, the study computed the optimal (i.e., the shortest) and top five sub-optimal pathways (i.e., longer than the shortest path) between all crRNA bases and the Cas13a residues, obtaining a distribution of communication efficiency in terms of the sum of edge betweennesses (i.e., the number of shortest pathways that cross the edge, measuring the “traffic” passing through them). Path lengths were defined as the number of nodes/residues connecting the intended pair. This provided a comprehensive overview of all communications, constituting the crosstalk noise between crRNA and protein. Then, the study computed the optimal and sub-optimal pathways communicating two regions of the crRNA spacer (i.e., “seed” and “switch”), which have been reported to be critical for Cas13a activation (see Cell Rep., 2018, 24, 1025-1036), with the HEPN1-2 catalytic residues (R472, H477, R1048, H1053). This represents the signal of the interest (FIG. 2, Panel B). The result shows that in the crRNA-Cas13a (FIG. 2, Panel B, upper panel), broad noise distributions overlap with the signals, dampening the SNR compared to the tgRNA-Cas13a (FIG. 2, Panel B, middle panel). In the tgRNA-Cas13a, amplification of signals from the “switch” region indicates that tgRNA binding improves the crosstalk efficiency between the crRNA spacer and the HEPN1-2 catalytic residues. This observation agrees well with previously reported biochemical data, suggesting that the complementarity at the “switch” region could trigger the allosteric activation of HEPN1-2, with mismatches in this region preventing LbuCas13a activation. In the atgRNA-Cas13a (FIG. 2, Panel B, lower panel), there is also an improvement in communication for signals sourcing from the “switch” region.

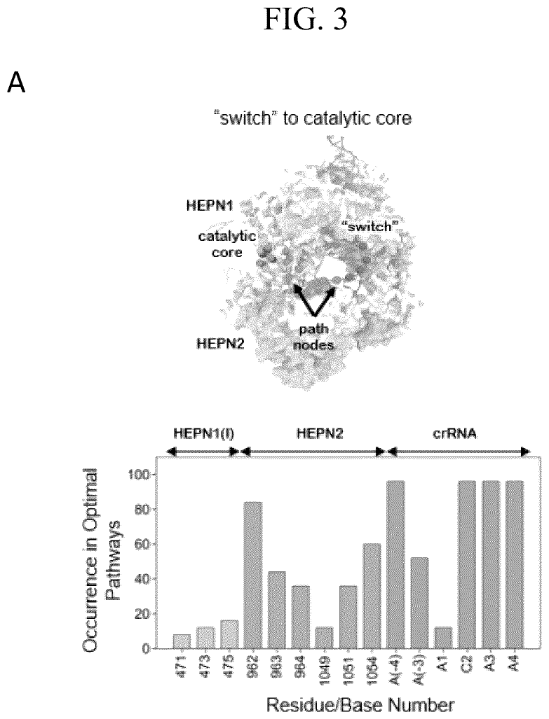

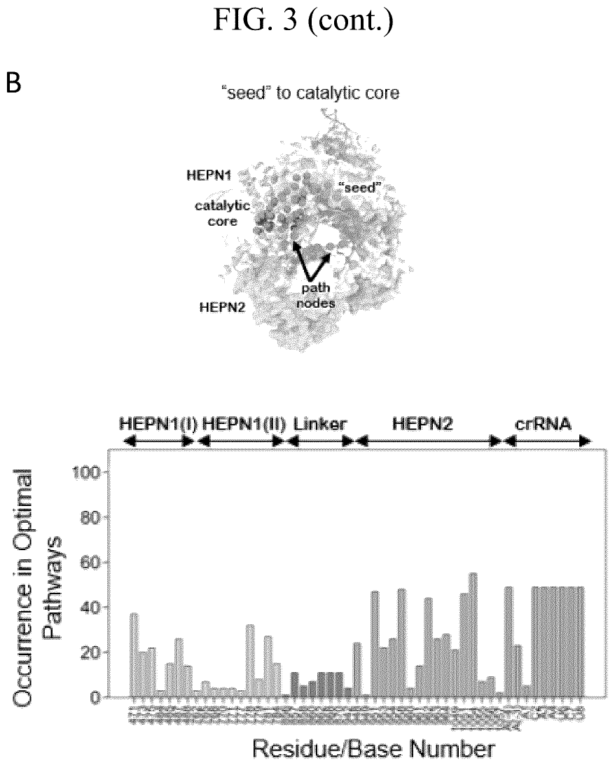

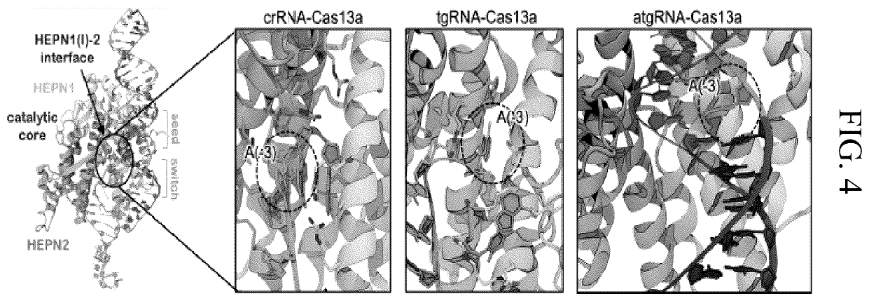

To further understand the signal transduction mechanism, the study computed the signaling pathways (i.e., the optimal and top five suboptimal paths) connecting the “switch” and “seed” regions of the crRNA to the catalytic core residues of the tgRNA-bound complex (FIG. 3). The pathways connecting the “switch” region to the catalytic residues exclusively follow a route that directly connects the crRNA bases of the repeat region (A(−5)-C(−1)) to the catalytic core through the HEPN1(I)-2 interface (FIG. 3, Panel A). On the other hand, the “seed” region communicates with the catalytic core through multiple routes, involving the Linker-HEPN2 interface, the HEPN1(II) residues, as well as the HEPN1(I)-2 interfacial residues through the crRNA repeat, like the communication observed for the “switch” (FIG. 3, Panel B). Hence, the pathways connecting the “switch” nucleotides to the catalytic residues display a lower number of residues with increased occurrences, tracing a more efficient communication path, compared to the pathways connecting the “seed”. This observation further affirms the critical role of the “switch” in the allosteric activation of HEPN1-2.

To better understand the observations, the study analyzed the interactions between the crRNA repeat region (A(−5)-(C-1)) and the proximal HEPN1(I)-2 interface (residues: 371-383; 963-975). Notably, this interface region is located distally with respect to the catalytic cleft (FIG. 4). The observed that in the tgRNA-bound system, the A(−3) base of the crRNA repeat region penetrates the interface, hampering interactions between HEPN1(I) and HEPN2 residues. On the other hand, in the crRNA-Cas13a, the A(−3) base is flipped out of the junction A(−3) base allowing increased interactions between HEPN1(I) and HEPN2 residues, with respect to the tgRNA-bound system. In the atgRNA-Cas13a, the crRNA repeat region is sequestered due to the extended tag-anti-tag complementarity.

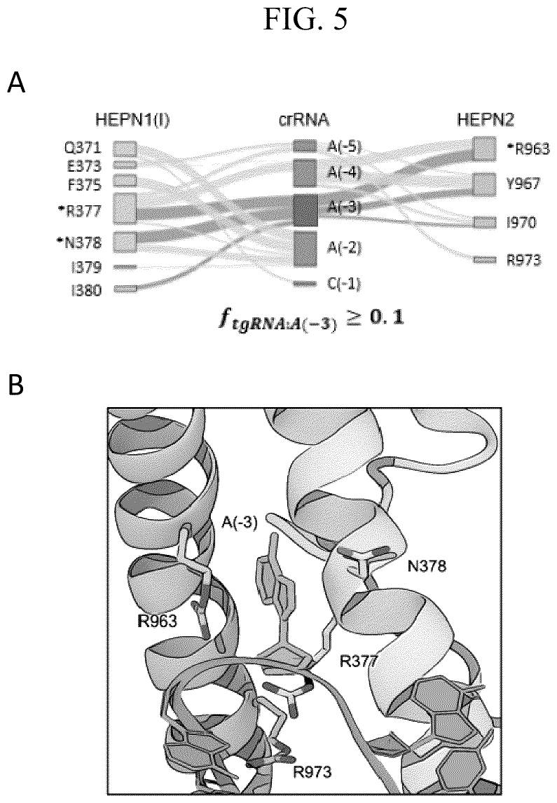

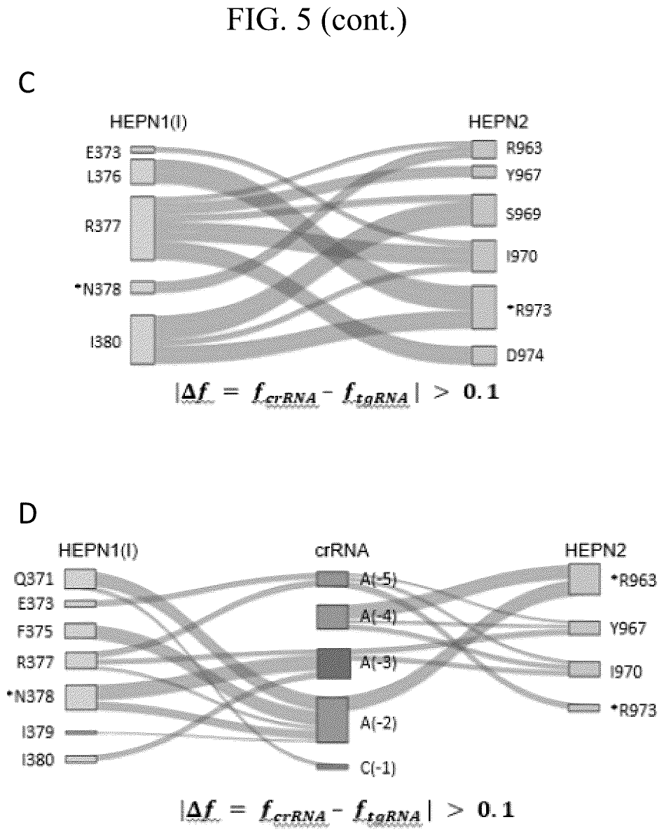

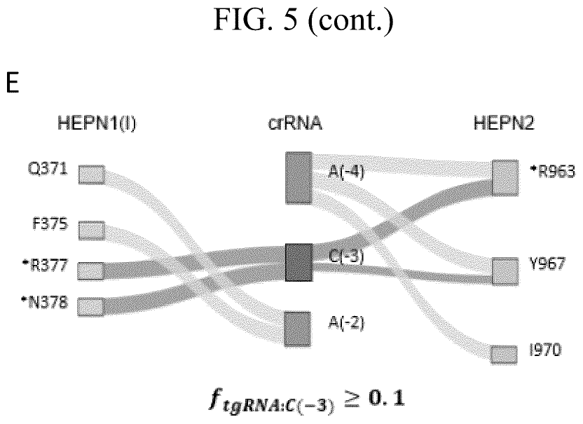

To detail the interactions of the A(−3) base in the tgRNA-bound Cas13a, the study conducted an in-depth contact analysis at the HEPN1(I)-2 interface (FIG. 5). A Sankey plot was used to report the frequency, f, of stable contacts between residues of the HEPN1(I) and HEPN2 domains, and the crRNA, forming for ≥10% of the simulation time (f≥0.1) in the tgRNA-Cas13a (FIG. 5, Panel A). In this plot, residues are connected through edges, whose width is proportional to f. The study observes that A(−3) substantially interacts with polar/positively charged residues, mainly R377, N378, and R963 (FIG. 5, Panel A, Panel B). The study also computed the differential contact stability to detail the gain or loss of contact stability moving from one system to another (e.g., crRNA-Cas13a vs. tgRNA-Cas13a). The difference in stability of contacts (Δf_{A-B}) in systems A and B, is computed as Δf_{A-B}=f_{A}−f_{B}, where the stability of contacts in systems A and B is represented by their frequencies, f_{A} and f_{B}, respectively. As f varies from 0 (contacts never formed) to 1 (contacts accounted in all frames), Δf_{A-B} varies from −1 to +1, where a Δf_{A-B}<0 corresponds to contacts relatively more stable in system B and Δf_{A-B}>0 corresponds to contacts relatively more stable in system A. Sankey plots were used to report Δf_{A-B} to characterize interactions that gain stability in tgRNA-Cas13a, compared to the crRNA-Cas13a. As expected, a loss of stable contacts at the HEPN1(I)-2 interface is observed in the tgRNA-Cas13a, compared to the crRNA-bound crRNA system (FIG. 5, Panel C). Upon tgRNA binding, R377 and N378 of HEPN1(I) gain interactions with A(−3); and R963 and R973 of HEPN2 increase their contacts with the neighboring bases, compared to the crRNA-Cas13a (FIG. 5, Panel D). To test whether this observation is sequence-dependent, the study substituted the A(−3) base with a smaller cytosine in the tgRNA-Cas13a and carried out an additional ˜5 μs-long simulation. In this system, the C(−3) base is stably located at the HEPN1(I)-2 interface and interacts with R377, N378, and R963 (FIG. 5, Panel E). Taken together, these observations suggest that these rearranged interactions involving charged/polar residues between HEPN1(I)-2 could be critical for allosteric signaling from the crRNA spacer to the catalytic core.

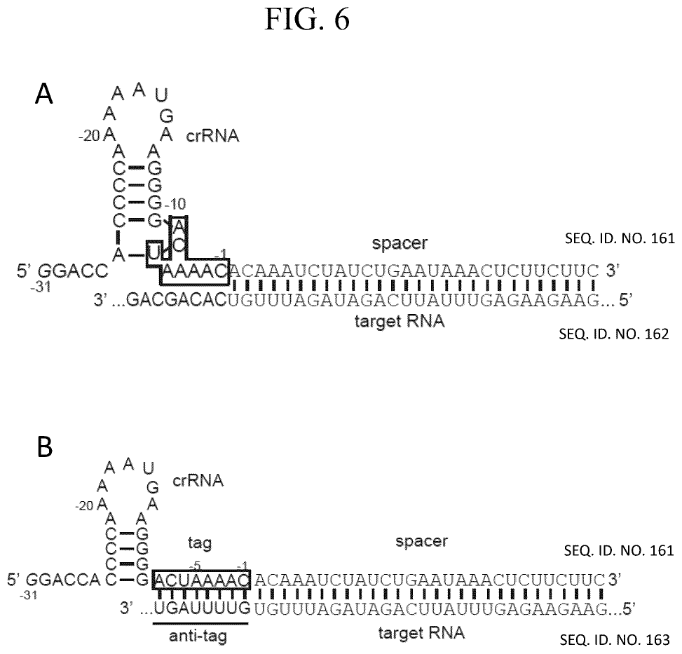

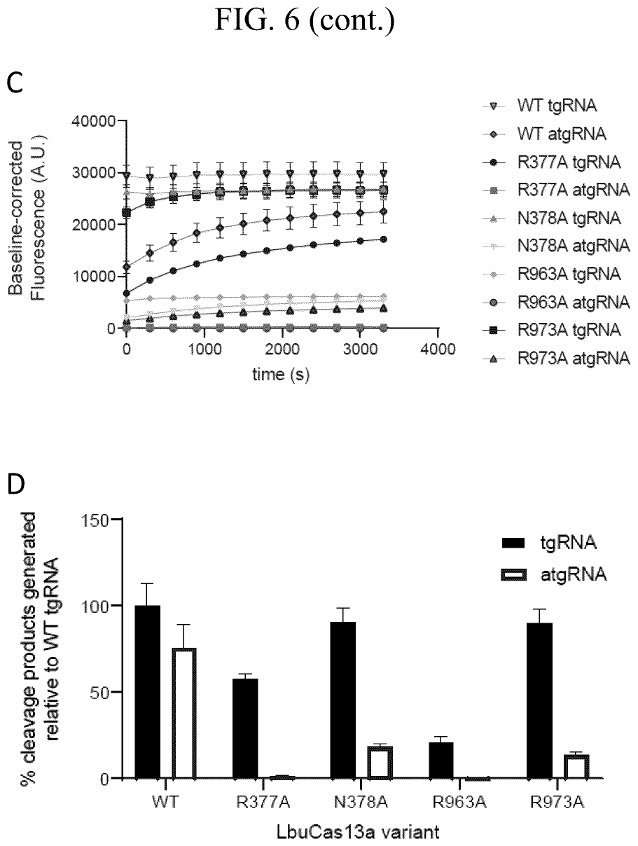

To experimentally verify the observations, the study generated and purified the wild-type (WT) Cas13a and four variants, mutating charged/polar residues to alanine at the HEPN1(I)-2 interface (i.e., R377A, N378A, R963A, and R973A, FIG. 5, Panel B). The study designed and generated tgRNAs and crRNAs containing the spacer used by Liu et al. (Cell, 2017, 170, 714-726) (PDB: 5XWP, FIG. 6, Panel A) and an anti-tag containing target RNA (atgRNA), holding an extended 8-nt. sequence with complementarity to the crRNA direct repeat (FIG. 6, Panel B). The study performed fluorescent RNA trans-cleavage assays with WT LbuCa13a and the variants bound to the tgRNA and atgRNA sequences. In the WT LbuCas13a, there is robust activation of the nuclease activity with a tgRNA, while the presence of an atgRNA results in a small decrease in the apparent cleavage rates over time (FIG. 6, Panel C), albeit the amount of end-point cleavage product was only slightly reduced relative to the tgRNA (FIG. 6d). In the LbuCas13a variants, N378A and R973A maintained a robust activity for the tgRNA, the R377A variant suffered a reduction in cleavage efficiency and R963A was not active at all (FIG. 6, Panel D). In the presence of an atgRNA, none of the variants displayed any significant nuclease activation, suggesting that these variants are more sensitive to anti-tag containing RNAs than the WT LbuCas13a (FIG. 6, Panel C, Panel D).

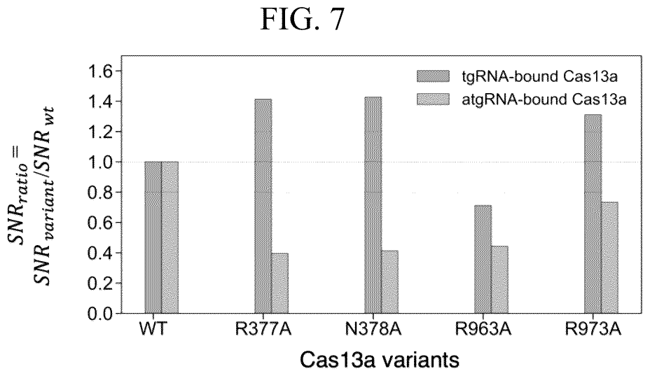

To evaluate how the signaling transfer is affected by the mutations the study made, the study collected a ˜15 μs ensemble for each of the four Cas13a variants bound to a tgRNA and atgRNA and compared it to the WT Cas13a. The study then analyzed the signaling transfer by comparing the SNR detected in the variants with that of the WT Cas13a, considering all signals sourcing from the critical “switch” region, and establishing a consistent scale for comparison (FIG. 7). The study thereby computed the ratio between the SNR in the variants and in the WT Cas13a (SNRᵣₐₜᵢₒ=SNRᵥₐᵣᵢₐₙₜ/SNR_{wt}). This comparison indicates whether point mutations at the HEPN1(I)-2 interface impact the strength of the communication between the crRNA “switch” and the catalytic residues compared to the WT upon tgRNA or atgRNA binding. The study observed that in the tgRNA-bound systems, the R377A, N378A, and R973A variants maintain a high SNRᵣₐₜᵢₒ, as evidence of efficient communication compared to the WT Cas13a (FIG. 7). On the other hand, R963A reduces the signal with respect to the WT, indicating altered communication. Upon atgRNA binding, the SNRᵣₐₜᵢₒ reaches 40-60% of perturbations in all variants with respect to the WT, with R973A maintaining a SNRᵣₐₜᵢₒ approximately within 30% of the WT. This suggests that, in the atgRNA-bound variants, the signaling from the “switch” to the HEPN1-2 catalytic core is reduced. This is in line with the experimental activity, showing that none of the variants displays a significant nuclease activation in the presence of an atgRNA (FIG. 6, Panel C, Panel D).

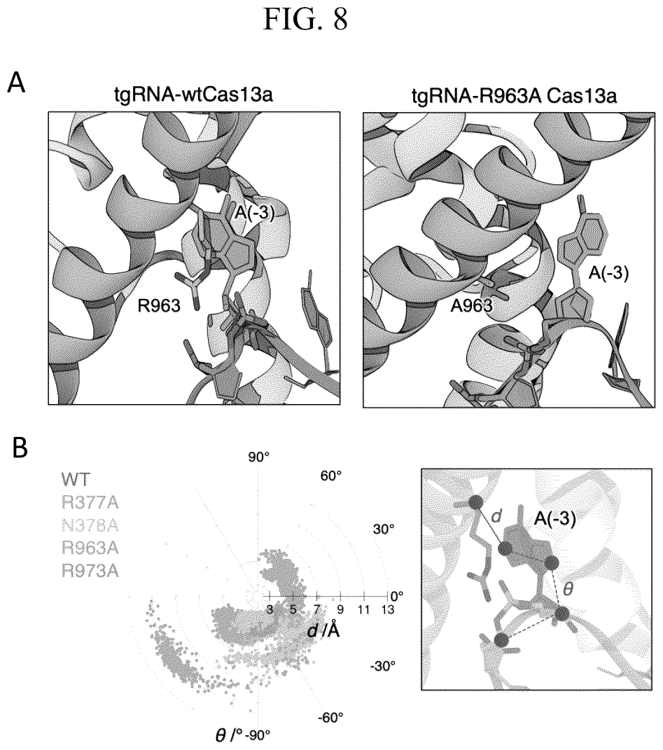

To further understand the signaling transfer in the tgRNA-bound variants, and the observation of a reduced SNRᵣₐₜᵢₒ in the inactive R963A mutant, the study analyzed the interactions of the crRNA repeat bases and the proximal HEPN1(I)-2 interface. In the WT tgRNA-Cas13a, the A(−3) base forms stable contacts with R377, N378, and R963, along with several other HEPN2 residues (FIG. 5, Panel A). These interactions are preserved in the tgRNA-bound mutants except, as expected, for the mutated residue. Nevertheless, in the tgRNA-bound R963A mutant, the A(−3) base is more flexible and is frequently extruded from the HEPN1(I)-2 interface (FIG. 8, Panel A). The A(−3) conformations are monitored on a polar plot reporting the distance d, which describes the displacement of A(−3) with respect to the Ca atom at position 963, and the dihedral angle θ, reporting the rotation of the A(−3) purine base with respect to the crRNA backbone (FIG. 8, Panel B). The polar plot provides evidence of increased flexibility of A(−3) in the R963A mutant, compared to the remaining systems, and its extrusion from the HEPN1(I)-2 interface. This observation can be ascribed to the loss of interaction between the R963 guanidinium side chain and the crRNA phosphate backbone that, on the other hand, is maintained in the other systems. As observed, the R963A substitution hampers the tgRNA-Cas13a activity (FIG. 6). This indicates that the positioning of the A(−3) base, and the dynamics of the crRNA repeat region at the HEPN1(I)-2 interface, critically affects the transmission of the signal from the “switch” to the catalytic core, as evidenced by lower SNR with respect to the WT.

In summary, the computational analysis characterized the allosteric activation mechanism of Cas13a and disclosed critical point mutations able to promote tgRNA-mediated over an extended tag-anti-tag-mediated complementarity, for RNA cleavage activation. It has been shown that the binding of a tgRNA acts as an allosteric effector of the spatially distant HEPN1-2 catalytic cleft, by amplifying the allosteric signals that connect the sites of tgRNA binding with the HEPN1-2 catalytic site. By introducing a novel graph theory-based analysis—Signal-to-Noise Ratio (SNR) metric of communication efficiency—the analysis show that the allosteric signal stands out over the dynamical noise when passing through the crRNA repeat region. Critical residues at this interface (R377, N378, and R973) rearrange their interactions upon tgRNA binding and are shown experimentally to select tgRNA, over an extended atgRNA, for RNA cleavages. Considering this selectivity, the study shows that the alanine mutation of R377, N378, and R973 can improve (or alter) the selectivity of Cas13a.

### Example 2: LbuCas13a Mutants have Higher Nuclease Specificity with Four Mismatches Between the crRNA and the Target RNA

Based on these observations, the study hypothesized that altering the allosteric communication pathway via mutational analysis could alter the mismatch tolerance profile of LbuCas13a, which could in turn facilitate the development of high-fidelity Cas13 enzymes. Structure-guided engineering of CRISPR enzymes has been successful in many cases which has yielded enzyme versions with high on-target cleavage and minimal off-target effects that could preclude its use as editing tools or therapeutic applications.

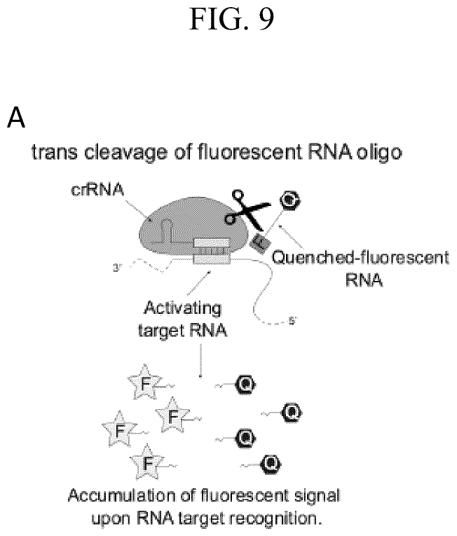

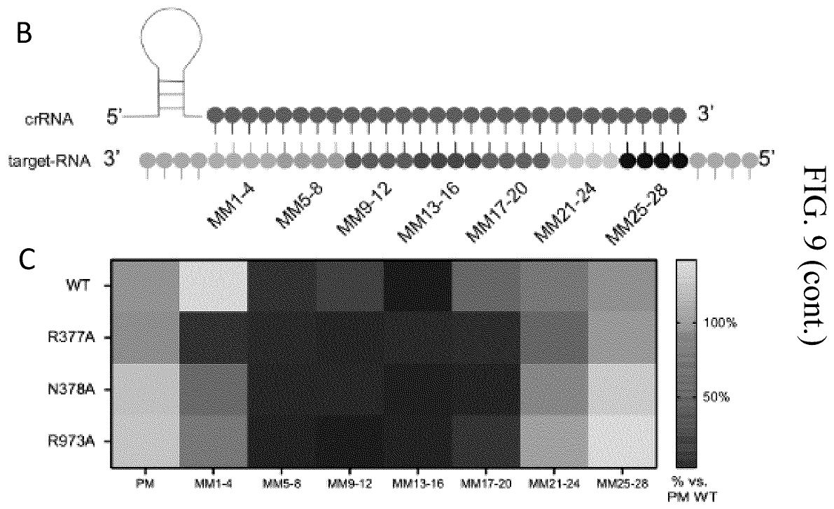

To test this hypothesis, the study performed biochemical experiments harnessing Cas13's trans-cleavage activity as a readout of Cas13 RNA-mediated HEPN-nuclease activation. Upon activation with a sufficiently complementary target RNA, Cas13 can non-specifically cleave quenched fluorescent reporters, resulting in increased fluorescent signal over time (FIG. 9, Panel A). This assay and similar strategies have been employed for the use of Cas13 as a very sensitive RNA-detection tool.

Cas13 shows differential activity and binding sensitivity to mismatches between the crRNA: target-RNA duplex in a position dependent manner. The study generated mutants via site-directed mutagenesis for each one of the selected residues as follows: LbuCas13aR377A, LbuCas13aN378A, LbuCas13aR973A. The study overexpressed and purified these proteins as described in [East-Seletsky, A., et al., Two distinct RNase activities of CRISPR-C2c2 enable guide-RNA processing and RNA detection. Nature, 2016. 538 (7624): p. 270-273]. To explore the contributions of each crRNA: target region for each of the Cas13a variants the study generated, the study performed trans-cleavage assays as previously reported in the presence of ssRNA molecules that are a perfect match to the crRNA, or ssRNAs with four consecutive mismatches tiling across the crRNA: target-RNA (FIG. 4, Panel B). The end-point fluorescence measurements after 1 hour showed that each one of these Cas13a variants are more sensitive to mismatches compared to the wildtype Cas13a protein (FIG. 9, Panel C). For example, while wild-type Cas13a produced robust and high nuclease activation with mismatches at positions 1-4 (relative to the crRNA) or 17-20, where several Cas13a variants show reduction or inhibition of nuclease activity if a mismatch occurs at these positions. These results suggest that the Cas13a variants have higher sensitivity to mismatches and therefore they might make excellent candidates for the development of high-fidelity Cas13 enzymes for RNA-detection applications.

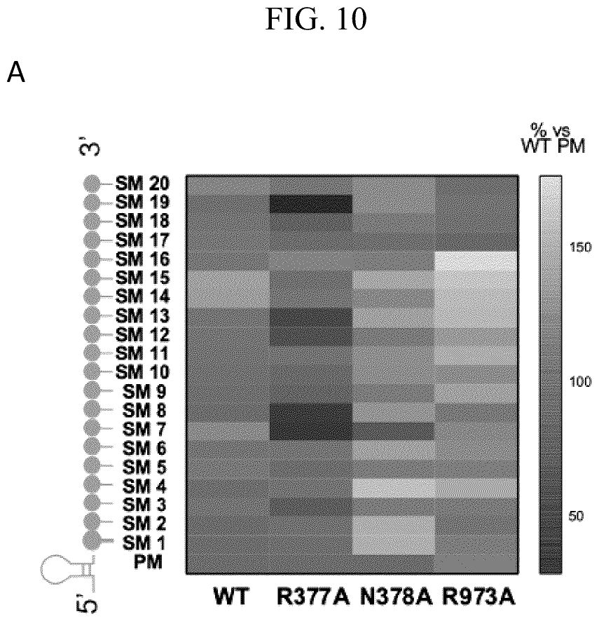

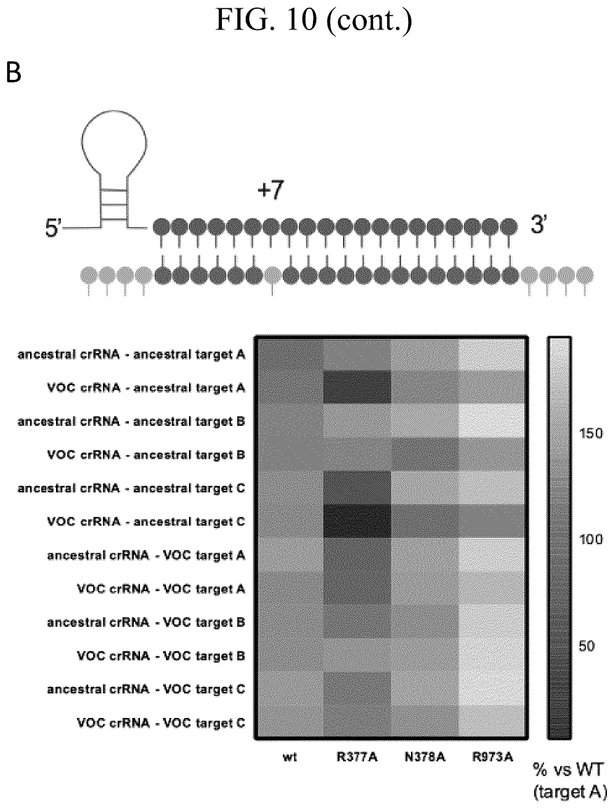

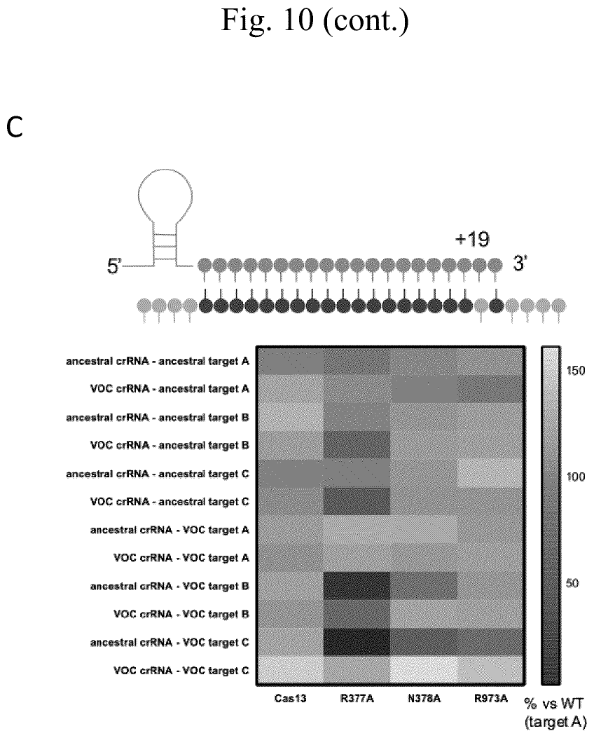

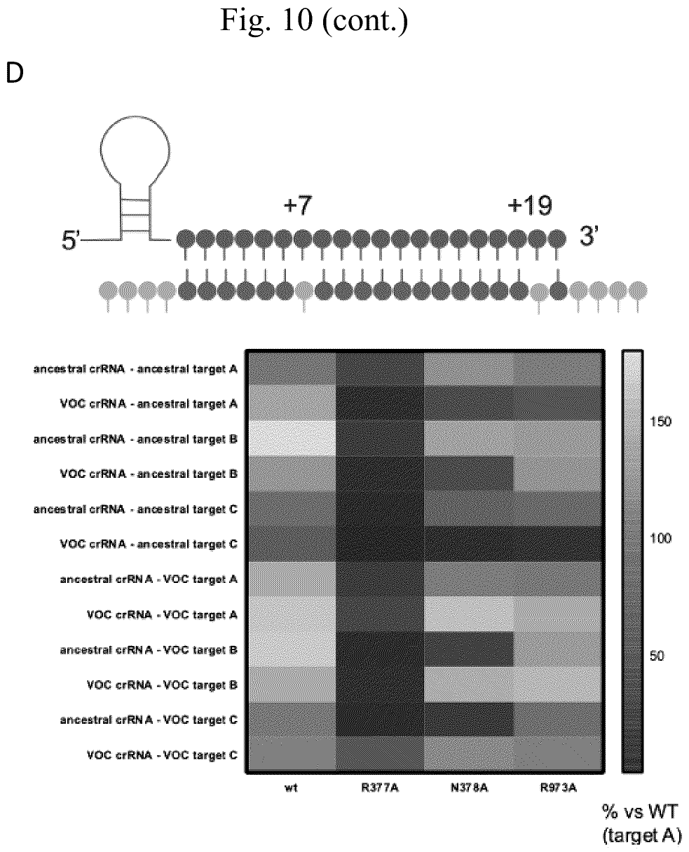

To further understand the contributions of each single position, the study synthesized RNAs with mismatches at each nucleotide position for every single position in the crRNA: target duplex. The study used 20 nucleotide crRNA-spacer lengths, as data in FIG. 9, Panel B show that this length is the most ideal for single nucleotide RNA discrimination. End-point normalized background-subtracted fluorescent values indicate that while the wild-type LbuCas13a shows no significant decrease in activity with ssRNAs with only single nucleotide mismatches at any position of the crRNA: target, the Cas13a variants show higher sensitivity (FIG. 10, Panel A). Mismatches in positions 7, 8, 12, 13, 19 are particularly sensitive and result in partial or complete inhibition of nuclease activity for LbuCas 13a^{R377A} and LbuCas13a^{N378A}, while retaining WT activity for a perfect complementary RNA (PM). LbuCas13a^{R973A} does not exhibit any significant decrease in activity with single mismatches, on the contrary, single mismatches are some positions seem to yield stronger activation.

Given that these variants are in fact more sensitive to mismatches—in one direction or another—the study sought to test their performance as single nucleotide polymorphism (SNP) detection assays, which could be deployed as a powerful diagnostic tool that can be used at point-of-care sites. This could include genetic testing, detection of aberrant gene expression, cancer detection, or epidemiological surveillance of pathogen's strains of concern. As a proof-of-concept, the study synthesized 57 nucleotide long RNAs that contain SARS-COV-2 sequences corresponding to regions in the Spike(S) protein transcript, for which mutations have resulted in the spread of new highly contagious and virulent SARS-COV-2 strains. The study generated both the ancestral/Wuhan strain transcripts and S transcripts that correspond several variants-of-concern (VOC): Beta (D80A), Delta (L452R) and Omicron (S477N+T478K) strains. The study used crRNAs that were tailored to the ancestral strain or the VOC, such that mismatch(es) between the ancestral RNA and a VOC-specific crRNA (or vice versa) would result in discrimination by the study Cas13a variants, as measured by HEPN nuclease activation.

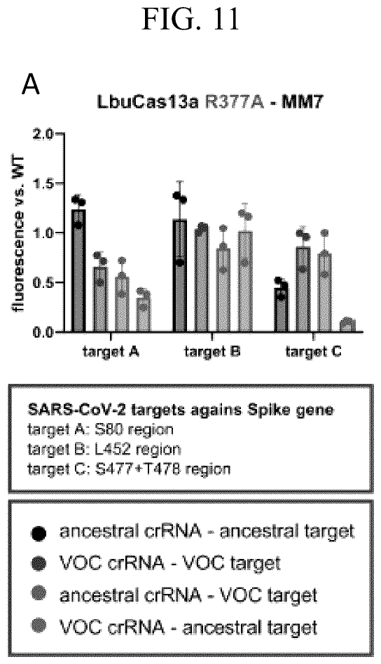

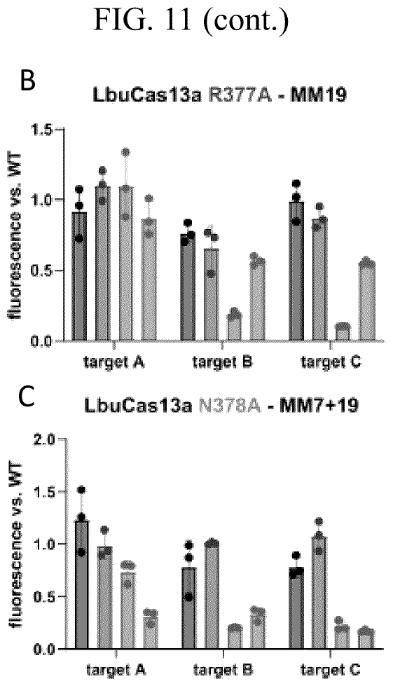

Given that mismatches at positions 7 and 19—relative to the crRNA—yielded significant decrease in nuclease activation, the study designed crRNA combinations where the SNP would occur at these positions. Using a mismatch at position 7 only resulted in limited discrimination for LbuCas13a^{R377A} and was not generalizable for every sample combination tested (FIG. 10, Panel B and FIG. 11, Panel A); other variants did not yield discrimination with this design (FIG. 10, Panel B). The presence of the SNP-detecting mismatch at position 19 resulted in about 50% decrease activity in LbuCas13a^{R377A} and in many samples it resulted in complete activation inhibition (FIG. 10, Panel C and FIG. 11, Panel B), whereas effects on LbuCas13a^{N378A} discrimination ability were modest (FIG. 10, Panel C). Additionally, the study tested an approach in which the study primed the enzyme with an initial synthetic mismatch at position 19 for all cases and then use position 7 for SNP detection. This approach resulted in strong inhibition for LbuCas13a^{R377A} (FIG. 10, Panel D). Importantly, introducing this synthetic mismatch yielded substantial discrimination in LbuCas13a^{N378A}, as shown by strong reduction in activity for most of the mismatched samples (FIG. 10, Panel D and FIG. 11, Panel C). This is particularly exciting as a combined use of variant LbuCas13a^{N378A} primed with an initial synthetic mismatch can readily be deployed for SNP detection. These results suggest this dual mismatch approach is the most promising for the development of novel high-fidelity Cas13 SNP diagnostics.

The study investigation on structure-guided engineering of Cas13a proteins has yielded enzymes that can be more versatile and easier to customize than any other Cas13 protein described to this date. Particularly, for SNP detection the study Cas13a variants are poised to achieve high levels of discrimination at the single-nucleotide level with straightforward crRNA design principles, which is unprecedented in the Cas13 diagnostics space.

Particularly, these enzymes display sensitivities at specific critical crRNA: target-RNA regions that the study have exploited for fine discrimination of virtually any set of RNA samples where SNP detection is the end-goal. Currently there are no user-friendly crRNA design principles that guarantee SNP detection, and any potential design must be determined using a case-by-case basis, which can complicate the use of this technology for SNP diagnostics, especially when rapid customization is required (e.g. during outbreaks).

In sum, here the study presents compelling evidence that the Cas13 variants the study generated by studying the RNA mediated allosteric activation of Cas13a are excellent candidates for highly specific detection tools, particularly for the detection of SNPs. While LbuCas13a^{R377A} and LbuCas13a^{N378A} could be applied for detection of SNPs, LbuCas13aR973A could be useful for assays in which you want relaxed specificity with higher than WT activity to detect several mutations at once. For example, one could use LbuCas 13a^{R973A} to detect any and all VOCs that may be present (i.e., able to detect across VOCs as a “pan” detection device).

### Example 3: Mismatch-Sensitive Hotspots in the Guide RNA

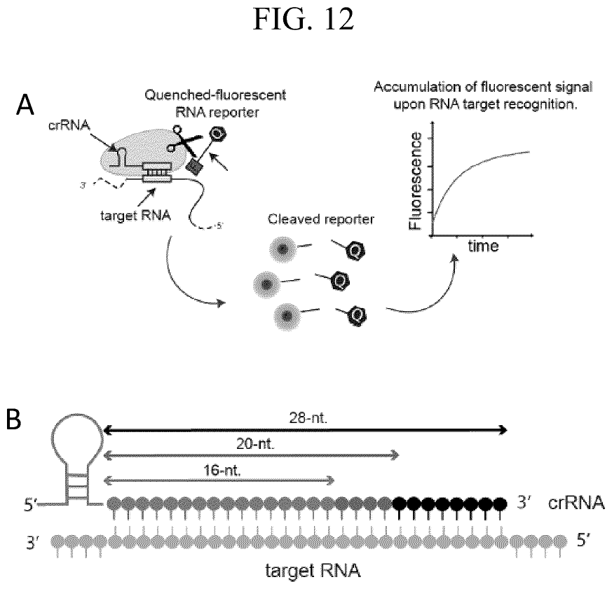

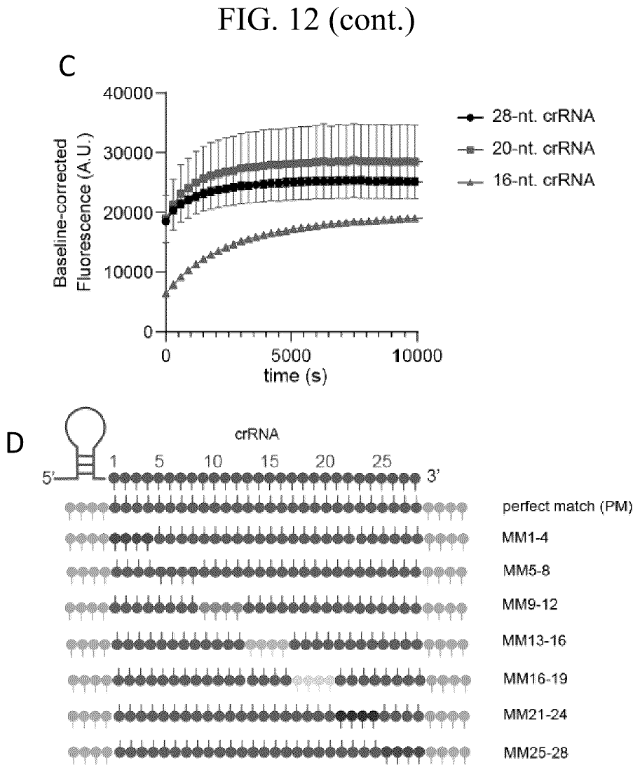

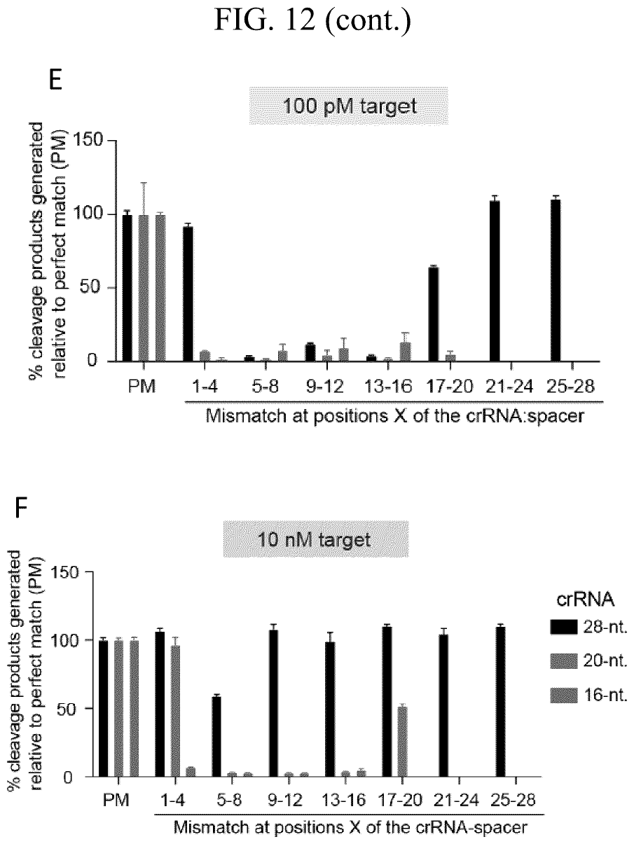

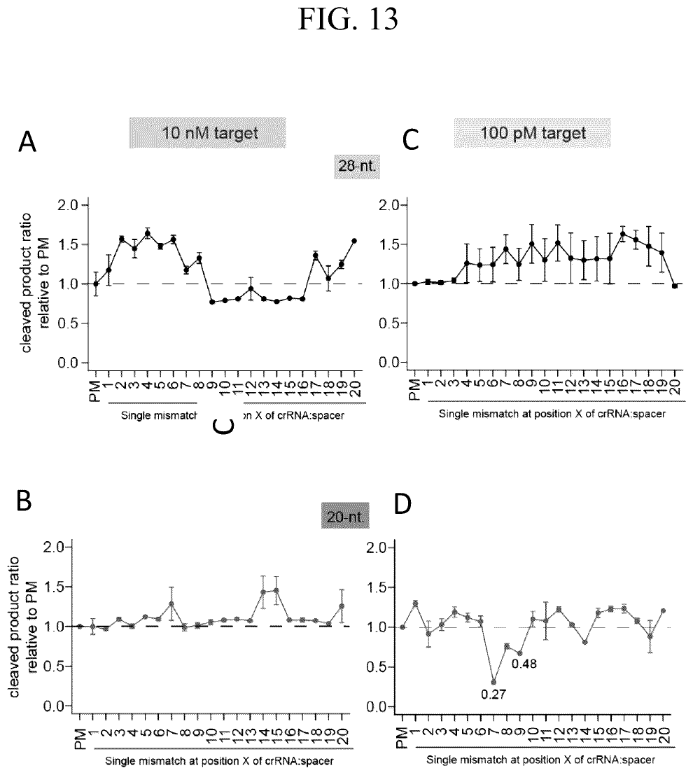

To study mismatch-sensitive hotspots in the guide RNA, the study harnessed Cas13's collateral-cleavage activity as a readout of Cas13 RNA-mediated HEPN-nuclease activation. Upon activation with a sufficiently complementary target RNA, Cas13 can non-specifically cleave quenched fluorescent reporters, resulting in increased fluorescent signal over time (FIG. 12, Panel A). The study first designed crRNAs targeting the same target-RNA with 16, 20 or 28 nucleotide (nt.) crRNA-spacer lengths (FIG. 13, Panel B) and showed that Cas13a exhibits robust activation with these three crRNAs, albeit the study observed a decrease in activity with a 16-nt. crRNA-spacer (FIG. 12, Panel C).

Given that LbuCas 13a nuclease activity was maintained across all crRNA-spacer lengths tested, the study then probed for sensitivity to mismatches at different regions of the crRNA-spacer: RNA-target region by introducing four consecutive mismatches across the complementarity region (FIG. 12, Panel D) and assessed the amount of cleavage product generated after one hour incubation with different RNA target concentrations. The study noticed that this previous study incorporated an additional adenosine nucleotide at the 3′ end of the direct repeat (DR) in the crRNA, which inadvertently introduces an additional mismatch between the spacer and the target RNA, and thus the study decided to revisit these experiments using the canonical LbuCas13a DR that does not include this additional adenosine. Of note, the mismatches in the target RNA were generated by replacing the nucleotide(s) in the target-RNA with the same nucleotide one present in the crRNA-spacer sequence, such that mismatch pair is made up of two of the same nucleotide. At 100 pM RNA target and targeting with a 28-nt. crRNA-spacer, ribonuclease activity could not be detected with mismatches between regions +5 to +13 relative the crRNA-spacer (FIG. 12, Panel E). Interestingly, with shorter 16 or 20-nt. crRNA-spacer only a perfectly matched target-RNA was able to activate LbuCas13a (FIG. 12, Panel E). The study tested the same panel of mismatched target-RNAs using higher target-RNA concentration (10 nM) and observed that most of these cleavage defects were rescued at this high concentration when using a 28-nt. crRNA-spacer, with only a slightly decreased activity in region 5-8. The 20-nt. crRNA-spacer maintained RNase activity when there were mismatches in the 1-4 or 17-20 regions (FIG. 12, Panel F), and the 16-nt. crRNA-spacer still required a perfect match for RNase activity (FIG. 12, Panel F). However, as the study saw in FIG. 13, Panel C, using a crRNA with a 16-nt. spacer comes at a cost of total fluorescence magnitude and time to reach plateau, which could undermine sensitivity and assay times for diagnostic applications. As a result, the study decided not to further pursue 16-nt. spacer design in this study.

Taken together, as expected, LbuCas13a displayed differential sensitivities to mismatches between the crRNA-spacer and its target-RNA in a position-dependent manner, and importantly, this sensitivity profile changes depending on the length of the crRNA-spacer used, with shorter crRNA-spacers being more sensitive to mismatches, and thus yielding higher specificity Cas13-crRNA complexes. It also cannot be overstated that higher concentrations of partially matched target RNA can in some cases still elicit nuclease activity, and this activity needs be considered when designing diagnostic assays, especially in context of pre-amplification of the RNA target, where fine control of the final concentration of target is difficult and can vary from sample to sample.

### Example 4: Effect of crRNA-Spacer Length on Mismatch Tolerance

To further assess the effect of crRNA-spacer length on mismatch tolerance, the study explored the effect that single nucleotide mismatches in the crRNA-target duplex have on Cas13 activation. The study generated RNAs that contained a single nucleotide mismatch at each one of the first 20 nt. positions of the spacer (using the canonical LbuCas13a DR), performed trans-RNA cleavage assays and assessed the relative cleavage efficiencies compared to a perfectly complementary RNA target. For crRNA-spacer lengths of 28 and 20 nucleotides, single nucleotide mismatches across the length of the spacer did not impact Cas13 RNase activity at high target concentrations (10 nM) (FIG. 13, Panels A-B).

When the study lowered target-RNA concentrations to 100 pM, 28-nt. still yielded high Cas13a activity regardless of the presence of single-nucleotide mismatches in the target RNA (FIG. 13, Panel C). On the other hand, when using a 20-nt. crRNA-spacer, single mismatches result in diminished RNase activation at several positions in the middle of the spacer (+7, +8, +9), and these mismatches resulted in up to a 70% reduction in activity, compared to a perfect-matched target-RNA under the same conditions (FIG. 13, Panel D). It should be noted that compared to Tambe et al., the location of mismatch-sensitive and tolerant regions was slightly different. In particular, this single mismatch profiling seems to indicate a subtle shift of one nucleotide in mismatch sensitivity, consistent with the different crRNA design.

Taken together, probing each base pair in the spacer: target-RNA duplex via mismatch analysis uncovers potentially useful mismatch sensitivities at specific regions of the spacer, however this is highly dependent on the length of the spacer as well as the target RNA concentration. In addition, 28-nt. spacers yield no single mismatch discriminatory capacity at the target-RNA concentrations tested.

### Example 5: Regions of the crRNA-Spacer: Target-RNA Duplex that are Most Important for Gating HEPN Nuclease Activation

The study determined that certain regions of the crRNA-spacer: target-RNA duplex are most important for gating HEPN nuclease activation, and further explored this potential allosteric coupling using computational approaches. Target RNA binding acts as an allosteric activator of the HEPN nuclease domains and identifies critical regions of LbuCas13a responsible for this information transfer. With this in mind, the study wondered whether the differential, position-dependent mismatch sensitivity observed with 20-nt. length crRNA-spacers is also associated with perturbed allosteric coupling between the spacer nucleotides and catalytic residues of LbuCas13a. The study conducted similar molecular dynamics (MD) simulations of the LbuCas13a complexes with single mismatches introduced at different locations within a 20-nt. spacer. MD simulations were carried out on four Cas13a: crRNA: target-RNA complexes containing either a perfectly matched crRNA: target-RNA duplex (PM), or crRNA: target-RNA duplexes that contain a single mismatch at either spacer nucleotide position 4, 7, or 11. These positions were chosen either because they displayed a large loss of cleavage activity when mismatched (position +7) or no noticeable loss of cleavage activity (positions +4 and +11), as a negative control for the downstream analyses. Each system was simulated for ˜1 μs and in three replicates, collecting a 12-μs ensemble necessary for the analysis of the allosteric signaling.

The study conducted graph-theory based network analysis to characterize the allosteric pathways of communication in the presence of single mismatches and compared these with the system with a perfectly matched crRNA-target RNA duplex. To estimate the communication efficiency between the crRNA spacer and the catalytic residues (R472, H477, R1048, H1053), the study employed a Signal-to-Noise Ratio (SNR) measure (see Material and Methods).

The SNR measures the preference of communication between predefined distant sites—i.e., the signal—over the remaining pathways of comparable length in the network—i.e., the noise. The SNR thereby estimates how allosteric pathways stand out (i.e., are favourable) over the remaining noisy routes, with high SNR values indicating the preference for the network to communicate through the signal.

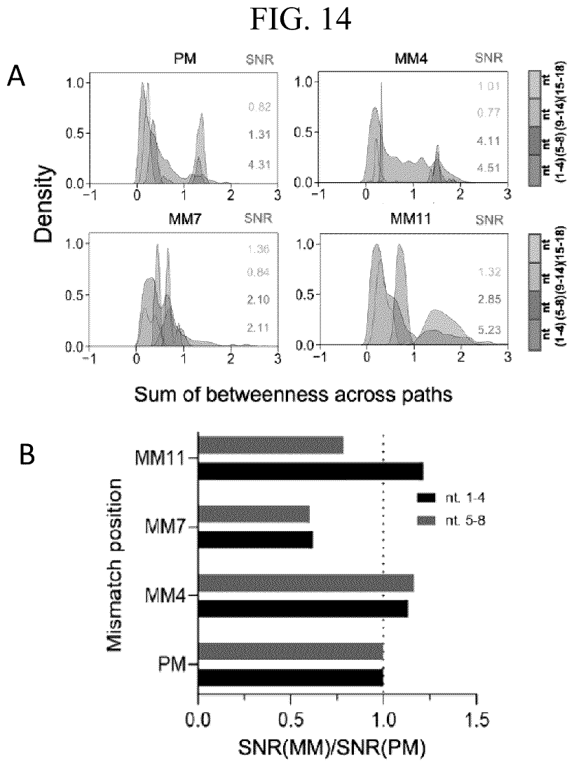

To detect crRNA-spacer regions with preferred communication with catalytic residues, the study performed SNR calculations considering signals sourcing from specific crRNA-spacer regions (i.e., nucleotides 1-4, 5-8, 9-14, and 15-18) and sinking to the catalytic residues (R472, H477, R1048, H1053) (FIG. 14, Panel A). The communication between all crRNA spacer nucleotides and all residues of the LbuCas13a protein was considered as the noise for all SNR calculations. As SNR calculation is sensitive to the pathlength of communication, which refers to the number of edges involved in the pathways connecting the spacer nucleotides with the catalytic residues, the study evaluated SNR across different pathlengths. Specifically, the study characterized the SNR for shorter (edge count: 6-8), medium (edge count: 9-11), and longer paths (edge count: 12-14) (see Materials and Methods). The obtained SNR along any of the paths assesses the prevalence of the signal over the noise by determining the extent to which the signal distribution differs from the noise distribution. The perfectly matched crRNA-spacer system exhibits relatively higher SNR values for spacer 1-4-nt. along shorter and medium-length paths, as well as for 5-8-nt. along longer paths, compared to other regions.

To facilitate comparison, the study compared the highest SNR observed in the no mismatch system with the highest SNR observed in the single mismatched systems, specifically corresponding to 1-4-nt. and 5-8-nt (FIG. 14, Panel B). This comparison indicates whether the introduction of single mismatches has impacted the strength of communication between the spacer and the catalytic residues compared to the system without mismatch. The ratio between the highest SNRs in the single-mismatched and PM systems reveals that mismatches at positions 4 and 11 still result in a similar level of communication as the no-mismatch system, with perturbation within 20% of this system. In contrast, introducing a mismatch at position 7 results in a ˜40% reduction in SNR compared to no mismatches, both for 1-4-nt. and 5-8-nt. These results suggest that mismatches at position 7, which impact LbuCas13a nuclease cleavage rate, result in a loss of allosteric crosstalk between the spacer and catalytic residues, unlike the mismatches at 4 or 11 which do not result in a loss of cleavage activity experimentally nor a large loss in allosteric crosstalk in these simulations.

### Example 9: Increased Sequence Specificity of Cas Enzyme Variants

Several different successful strategies to increase the sequence specificity of CRISPR-Cas enzymes have been employed in the past, including weakening the enzyme-target DNA interactions or slowing cleavage rates. The net effect is that the increasing difference between the dissociation and catalytic constant rates, allows the dissociation of non-perfect targets to be more favorable than cleavage, thus increasing discrimination. Applying this kinetic rationale, structure-guided engineering of CRISPR-Cas enzymes has yielded variants with high on-target cleavage and minimal off-target effects that improve their safety profile when used in research or therapeutic applications. Given the observations above and that the key R377, N378, and R973 residues gate the RNA-mediated allosteric HEPN activation, the study hypothesized that by altering these allosteric communication pathways the study could also alter the mismatch tolerance profile of LbuCas13a which, in turn, could facilitate the development of higher-fidelity Cas13 enzymes.

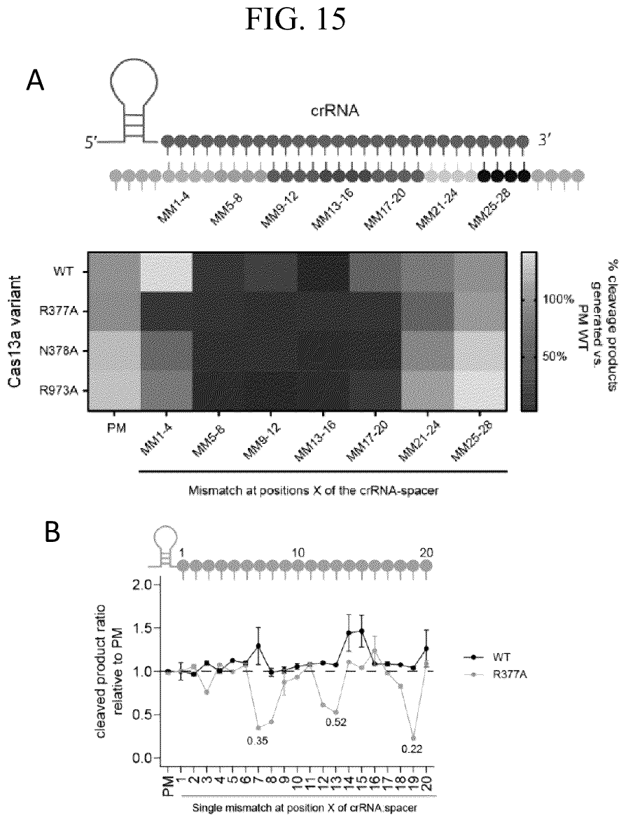

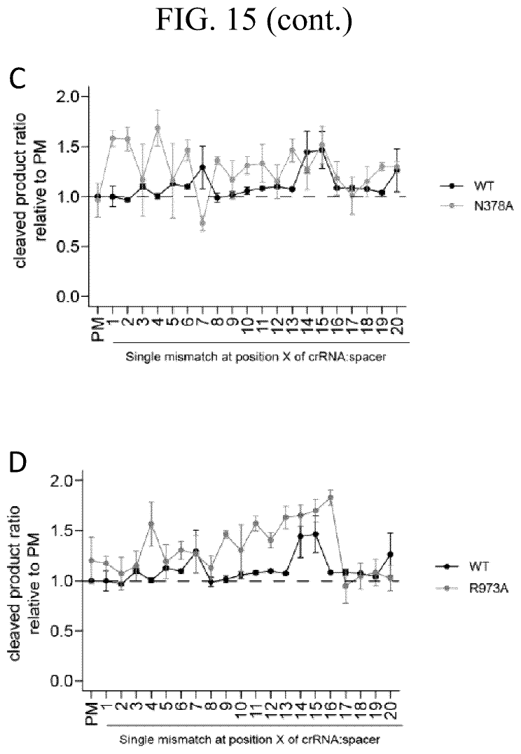

Specifically, the study found that variants LbuCas13aR377A, LbuCas13aN378A, LbuCas13aR973A have altered allostery communication pathways and the study sought to explore the mismatch tolerance across the crRNA-spacer for each of these Cas13a variants. To this end, the study overexpressed and purified these proteins and performed cleavage assays either in the presence of a perfect matched target-RNA to the 28-nt. crRNA-spacer, or ssRNAs with four consecutive mismatches tiling across the crRNA-target RNA duplex (FIG. 15, Panel A). End-point background-subtracted fluorescence measurements after one hour showed that each one of these Cas13a variants is more sensitive to mismatches compared to wild-type Cas13a (FIG. 15, Panel A). For example, wild-type Cas13a still exhibited robust nuclease activation with mismatches at positions 1-4 or 17-20, where all Cas13a variants tested showed a significant reduction in nuclease activity with these mismatched target-RNAs. No significant change in apparent cleavage efficiency occurred in regions 21-24 and 25-28 when mismatches were present. These results suggest that the Cas13a variants have higher sensitivity to mismatches and thus may make suitable candidates for the development of higher-fidelity Cas13 enzymes for RNA-detection applications.

Given, the study saw no sensitivity to mismatches in regions 21-28 (FIG. 15, Panel A) and the data in FIG. 11, Panel E suggest that 20-nt. spacers are most appropriate with respect to generating new more specific Cas13 variants with single-nucleotide discrimination potential. To this end, the study wanted to further understand the contributions of each single nucleotide in cleavage efficiency, and the study used the previously generated RNAs with mismatches at each nucleotide position for every single position in the crRNA-target duplex. End-point normalized background-subtracted fluorescent values indicate that the LbuCas13a^{N378A} and LbuCas13a^{R973A} variants show higher sensitivity to single-nucleotide mismatches from 100 pM ssRNAs with 20 nucleotide spacers and display a more pronounced loss of activity profile across the crRNA-target compared to the wild-type LbuCas13a.

Remarkably, LbuCas13a^{R377A} is not able to activate with the low 100 pM ssRNA concentration but at higher concentrations of ssRNA, activity with perfect-matched RNA is comparable to wild-type but single-pair mismatches at positions 7, 8, 12, 13, 19 within the crRNA-target RNA duplex particularly sensitive and is sufficient to result in loss of nuclease activity (FIG. 15, Panel B). At this concentration, on the other hand, variants LbuCas13a^{N378A} and LbuCas13a^{R973A} do not exhibit any significant decrease in activity with single mismatches, and on the contrary, single B mismatches at some positions seem to yield more robust activation (FIG. 15, Panels C and D). The study applied the same approach to 28-nt spacers and found that regardless of target concentration, 28-nt. crRNAs do not show sufficient discrimination of mismatched RNAs.

Thus, altering residues that participate in the allosteric communication involved in HEPN nuclease activation gives rise to enzyme variants that display higher sensitivities to single nucleotide mismatches, although the degree of discriminatory power is position and spacer-length dependent. If using 20-nts. spacers, LbuCas13a^{N378A} and LbuCas13a^{R973A} variants are excellent at discriminating single-nucleotide mismatches at certain positions (mainly 7 and 19) when RNA target concentrations are low. If concentration of target is expected to be high, then LbuCas13a^{R377A} might make a strong candidate to distinguish single-nucleotide differences at those same positions.

### Example 10: Modification of the Handle Region of the Guide RNA

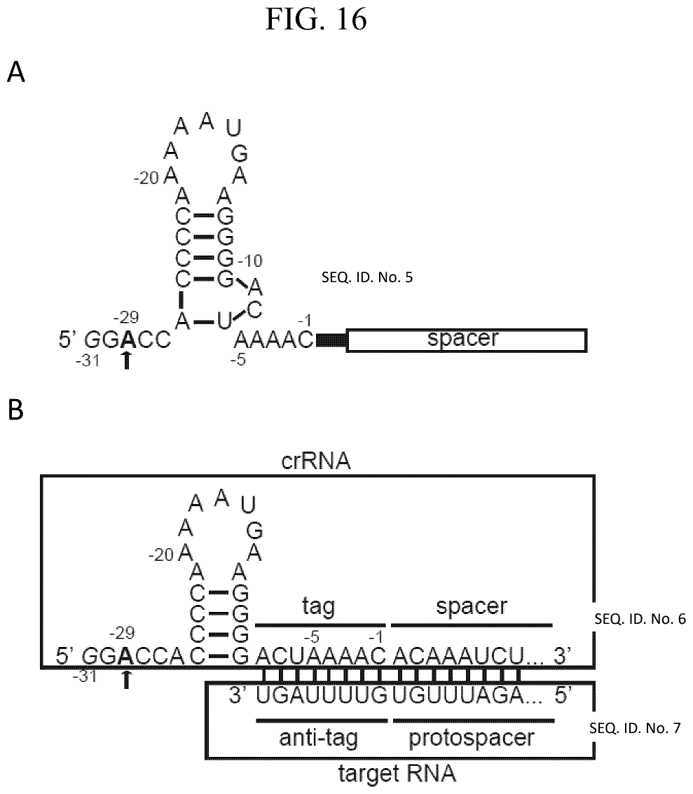

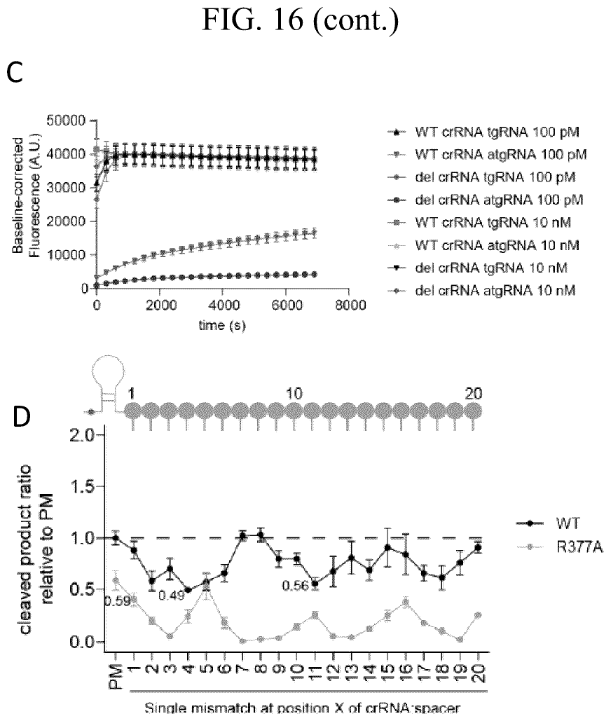

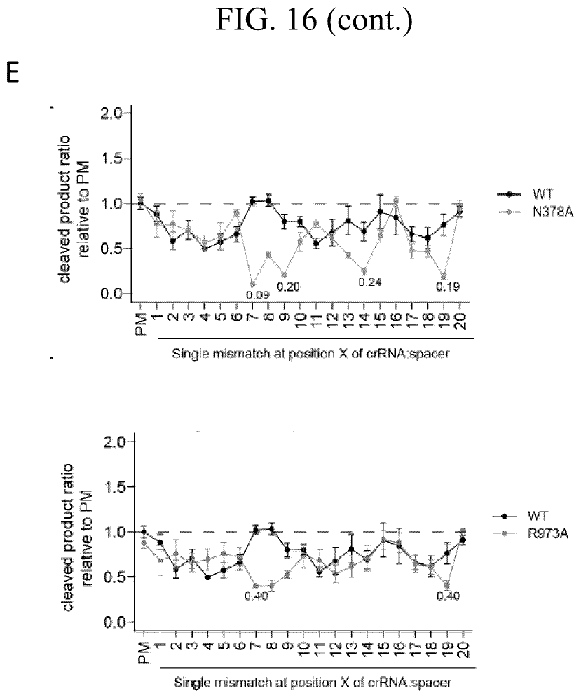

The crRNA DR sequence of ‘anti-tag’ RNA-mediated nuclease inhibition of Cas13a contains a deletion in the first adenine in the DR compared to other crRNA DR sequences commonly used for LbuCas13a (FIG. 16, Panel A). Extended complementarity of the target RNA (forming an ‘anti-tag’) with the direct repeat of the crRNA of about 8 nucleotides results in inhibition of Cas13a activity despite having perfect complementarity in the spacer (FIG. 16, Panel B). The study wondered if this deletion in the DR might contribute at least partially to the observed inhibition of Cas13 cleavage in the presence of anti-tag containing RNAs. The study changed the flanking regions of the target RNA with an anti-tag sequence for LbuCas13a crRNA (atgRNA) and used the original sequence without anti-tag (tgRNA). Additionally, the study designed the same crRNAs as previously used but containing the A-29 deletion (del crRNA) and compared its activity relative to the full-length DR containing crRNA (WT crRNA). The study then performed the trans-cleavage reporter assay in the presence of these crRNAs and targets (with and without anti-tag). The apparent cleavage efficiencies suggest that performing this truncation makes LbuCas13a more sensitive to inhibition by anti-tag containing RNAs, especially with low concentrations of target (100 pM) where the apparent cleavage activity is greatly reduced (FIG. 16, Panel C). Higher concentrations of target RNA can overcome this inhibition.

Conversely, when using the crRNAs and sequences reported by Meeske and Marraffini, and restoring the DR to full length, the nuclease activity in the presence of high anti-tag RNA concentrations is rescued to the same levels as the RNA target. Additionally, it appears that the deletion results in decreased nuclease activity even with targets that do not contain an anti-tag sequence.

While there might be sequence dependent effects mediating Cas13 activation, these assays suggest that truncating the direct repeat causes a subtle activation defect in Cas13a and makes it more sensitive to inhibition by anti-tag RNAs. Given the additional penalty imposed by this crRNA design, the study hypothesized that using this truncated crRNA architecture may have an impact in the mismatch tolerance profile of LbuCas13a, increasing its mismatch discrimination ability. To investigate this, the study performed additional cleavage assays with single-nucleotide mismatched target RNAs, for both wild-type LbuCas13a and the variants the study investigated above, loaded with a truncated crRNA. For spacer lengths of 20-nt., a single nucleotide mismatch had greater impact on WT Cas13a activity at many positions across the crRNA-target region when the crRNA is truncated, even at high (10 nM) target RNA concentrations (FIG. 16, Panels D-F). At low (100 pM) target concentrations, the relative cleavage activity on WT Cas13a with mismatched target RNAs is very low, resulting in near complete loss of activity in most cases, particularly in the middle region of the spacer, for example, positions 7-9 and 14. Combining this truncated crRNA with a 20-nt. spacer and the variant LbuCas13a enzymes, the discrimination between a perfectly matched RNA and a mismatched one is further improved in some cases (FIG. 16, Panels D-F). For example, LbuCas13aR377A activity with a perfectly matched target-RNA (at 10 nM) is decreased about 50% compared to its wild-type counterpart (FIG. 16, Panel D). Despite this apparent loss in activity, there is close to no activity of LbuCas13aR377A in the presence of mismatches at most positions, particularly 3, 7-9, 12-14, 19 at high target concentrations (FIG. 16, Panel D). For LbuCas13aN378A and LbuCas13aR973A, robust activation with mismatched target-RNAs at high target-RNA concentrations is mostly observed, with the exception of a few positions, mainly positions 7 and 19. For LbuCas13aN378A, the presence of these mismatches in positions 7 or 19 resulted in almost no signal (FIG. 16, Panel E), whereas for LbuCas13aR973A only a partial loss of activation is observed (˜50%) (FIG. 16, Panel F). Interestingly, at low concentrations of RNA target (100 pM), no nuclease activity is detected in any of the LbuCas13a variants even for a no mismatch RNA, suggesting a decrease in sensitivity across all these variants when in combination with this truncated crRNA. Together, the data suggest that combining a 20-nt. truncated crRNA with LbuCas13aN378A could be a promising candidate for the deployment of a much more specific Cas13-based diagnostic tool.

On the other hand, using 28-nt. spacers with a truncated DR with WT LbuCas13a does not result in single-nucleotide sensitivity, at either 100 pM or 10 nM target RNA. If using the LbuCas13a variants, LbuCas13aR377A is inactive at low target concentrations, even with a fully complementary RNA, but at higher concentrations (10 nM), LbuCas13aR377A is active and mismatches at positions 7 or 8 result in loss of cleavage activity that allows for single-nucleotide discrimination. For LbuCas13aN378A and LbuCas13aR973A, sensitivities at positions 7-10, 14 and 19 can be appreciated at 100 pM target RNA. Raising the concentration of target to 10 nM abolishes this discriminatory ability with no significant sensitivity at any position. If using 28-nt. crRNAs for diagnostic purposes is desired, combining the crRNA truncation with LbuCas13aR377A would likely be the most sensible approach if the RNA concentrations are expected to be high.

Additionally, the study used lateral flow readout to validate the potential for this discrimination approach to be adapted for point-of-care diagnostics. The study compared the perfect-matched RNA and one with a mismatch at position 7 and compared the lateral flow readout using WT LbuCas13a and LbuCas13aR377A with a full-length DR. Additionally using the truncated DR, the study compared the same target RNAs with WT LbuCas13a, LbuCas13aR377A and LbuCas13aN378A. In all cases, visual readout shows excellent discrimination using the new variants in all cases, unlike WT Cas13 that did not display sensitivity in the presence of mismatch 7 target RNA.

### Example 11: SARS-COV-2 RNA Detection

If performing SARS-COV-2 RNA detection, under pre-amplification conditions (closer to “real-life” conditions), strong discrimination with the LbuCas13a variants herein can be achieved vs. WT.

Shortening the crRNA/guide spacer (e.g., from 28-nt to 20-nt) allows for better discrimination of RNA mismatches at given positions, mainly 7 and 19 (relative to the crRNA). This better discrimination can be heightened in the LbuCas13a mutants. A strategy for single nucleotide mismatch discrimination for LbuCas13a is to design crRNAs where the discriminatory mismatch is located at those critical positions (e.g., 7 and 19). A deletion in the crRNA direct repeat (constant region of the crRNA), i.e., A(−29) allows for higher sensitivity to mismatches, especially for the LbuCas13a variants, which in turn means this crRNA variant can be deployed for more specific RNA diagnostics.

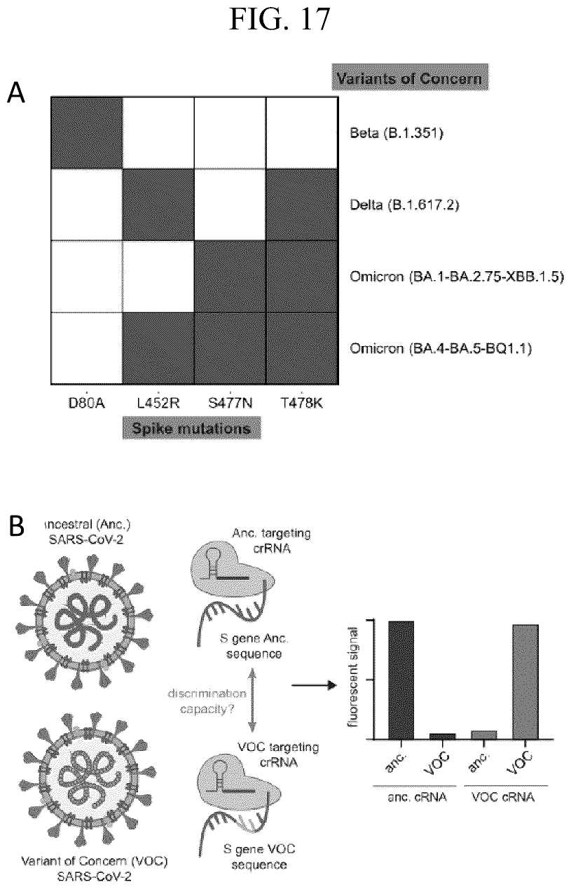

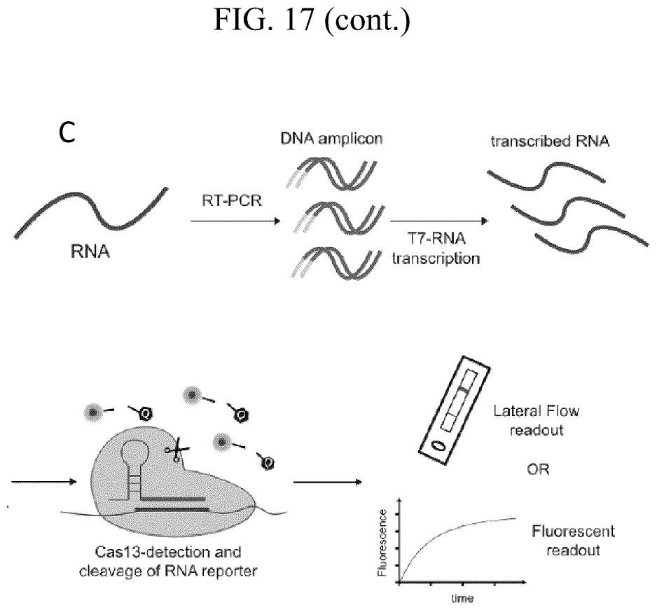

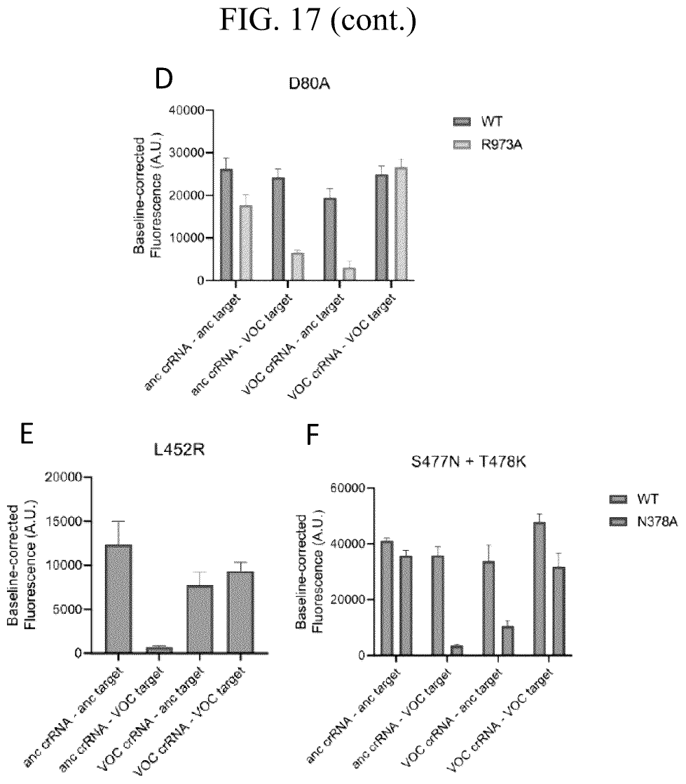

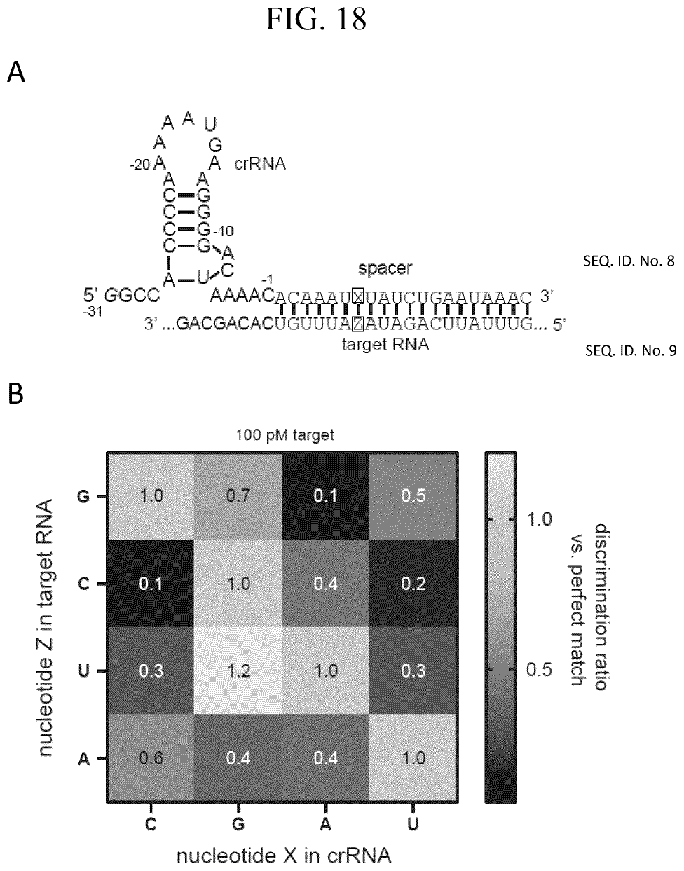

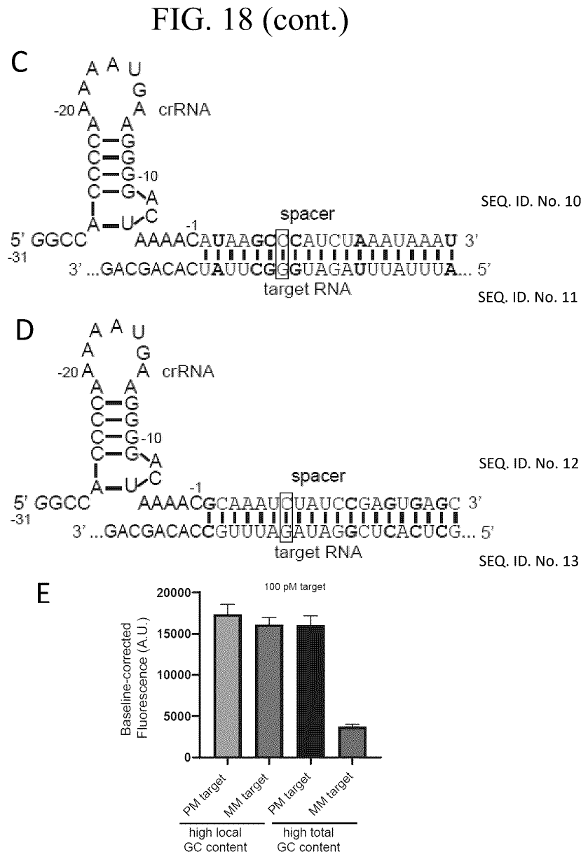

Given that together certain combinations of crRNA DR sequence variants and Cas13 protein variants are more sensitive to mismatches compared to WT LbuCas13a, the study sought to test their performance in single nucleotide polymorphism (SNP) detection assays, which in turn could be deployed as a powerful point-of-care diagnostic test. This test could include infection diagnosis, genetic testing, detection of aberrant gene expression, cancer-related SNP or gene fusion detection, or epidemiological surveillance of pathogens. As a proof-of-concept, The study designed assays against different regions of the Spike(S) protein transcript from SARS-COV-2, for which mutations in this gene have resulted in the spread of new highly contagious and virulent SARS-COV-2 strains. Particularly, the study looked at the following amino acid mutational hotspots in the S transcripts that are signature mutations of some variants-of-concern (VOC): Beta (D80A), Delta (L452R) and Omicron (S477N+T478K) strains (FIG. 17, Panel A). The study designed crRNAs that were tailored to the ancestral strain or the VOC, such that mismatch(es) between the ancestral RNA and a VOC-specific crRNA (or vice versa) would result in discrimination by the Cas13a variants, (FIG. 18, Panel B). The study first tested these crRNAs by transcribing short RNAs with these regions of interest and performing cleavage measurements using a range of different mismatch combinations, Cas13 variants and crRNA DR designs based on the positive data above and arrived at several strategies that can be used in some cases to carry out more robust SNP discrimination than using WT LbuCas13a protein.

The study then harnessed these crRNA design strategies for the viral discrimination from cultured viral extracts. To do this, the study employed a pre-amplification and detection strategy that couples amplification and T7 RNA polymerase transcription with the LbuCas13a cleavage assay and either a fluorescence or lateral flow read out to assay extracted RNA from SARS-COV-2 virus generated via cell culture (FIG. 17, Panel C). The study found that for the detection of the D80A mutation using LbuCas13aR973A and a crRNA (full DR) with a synthetic mismatch at position 19 and the discriminating mismatch at position 7 yielded strong recognition of the appropriate crRNA-target pairs but little activation in case the SNP of interest is present, unlike WT LbuCas13a that activates robustly in all cases (FIG. 17, Panel D). For the L452R-causing SNP, The study did not find a robust strategy as in these conditions the assay sensitivity seems to be low, but the study observed that using WT LbuCas13a and crRNAs with the discriminating mismatch at position 19, there is discrimination for an ancestral-designed crRNA and a VOC target (FIG. 17, Panel E). Finally, for Omicron variant detection (S477N+T478K) variant discrimination is achieved by using a crRNA where the discriminatory position is at nucleotide 19 for LbuCas13aN378A compared to WT LbuCas13a (FIG. 17, Panel F). Taken together, while the LbuCas13a SNP discrimination strategies are not completely generalizable yet, the data underscores that the addition of LbuCas13a variants and crRNA variants to the CRISPR diagnostics tool box offers an improvement in SNP discrimination ability compared to WT LbuCas13a and offers new opportunities for additional protein and crRNA engineering, and that sequence context likely plays a role in SNP discrimination power (see the next example for a further exploration of this idea) and thus multiple engineering strategies may be needed to enable easy to implement universal SNP discrimination.

### Example 12: The Influence of Interacting Mismatched Nucleotides and Nucleotide Content in the crRNA-Target Duplex on the Specificity on Mismatch Tolerance

The study noticed from the SARS-COV-2-like discrimination assays using short RNA targets that the degree of mismatch tolerance varies between the different RNA sequence targets tested in this study. Understanding whether there are additional crRNA or target RNA features that are contributing to these differences will allow researchers to make rational decisions about the most suitable crRNA design strategy to deploy for an RNA target of interest. The study hypothesized that the base pairs participating in the mismatch might be a contributing factor, as well as nucleotide content in the crRNA-target duplex (e.g., G-C content).

To test the influence of interacting mismatched nucleotides, the study chose the hyper-sensitive position 7 and obtained crRNAs and target-RNAs with the four possible nucleotides at that position and measured the relative cleavage efficiency, compared to a perfectly matched canonical base pair. Surprisingly, for either WT LbuCas13a at low target concentrations or LbuCas13a variants at high target concentrations, the study observed that different mismatched pairs elicit different activation patterns (FIG. 18, Panel A). For example, a C-C mismatch precludes Cas13 activation, but a G-G mismatch is very well tolerated, whereas G-A or C-U pairs have slightly different tolerances depending on their orientation in the crRNA-target.

Next, the study tested whether the G-C content of the target RNA contributes to differences in mismatch tolerance. From the original target sequence, the study derived two sequences: one with increased G-C content surrounding the sensitive position 7 but keeping the total original G-C content the same (25%) (FIG. 18, Panel B), and other sequence where the bases around the 7th position were maintained but the overall G-C content in the crRNA-target was raised from 25% to 50% (FIG. 18, Panel C). Performing trans-cleavage assays in the presence of a perfect match and a mismatch at position 7 of the crRNA: target-RNA duplex, revealed that raising the GC composition around the mismatch resulted in robust nuclease activation. On the other hand, preserving the sequence context but raising the global GC content, mismatch sensitivity is still maintained (FIG. 18, Panel D). Using higher concentrations of target RNA and the LbuCas13a variants yields similar differential tolerance based on sequence context and base pairs implicated in the mismatch. Taken together, the mismatch and sequence-context cleavage analysis reveal that the nucleotide engaging in a mismatch and the local sequence context surrounding the mismatch modulates mismatch tolerance and thus, the cleavage specificity.

List of Sequences

| SEQ | | |
|---|---|---|
| ID | | |
| NO: | Description | Sequence |
| 1 | Partial gRNA | 5′ |
| | sequence, | GACCACCCCAAAAAAUGAAGGGGACUAAAACACAAA |
| | artificial | UCUAUCUGAAUAAACUCUUCUUC 3′ |
| 2 | Target RNA | 5′ GGAAGAAGAGUUUAUUCAGAUAGAUUUGUC 3′ |
| | sequence, | |
| | artificial | |
| 3 | Partial gRNA | 5′ |
| | sequence, | ACCCCAAAAAAUGAAGGGGACUAAAACUAGAUUGCU |
| | artificial | GUUCUACCAGUAAUCC 3′ |
| 4 | Target RNA | 5′ GGAUUAACUGGUAGAACAGCAAUCUAGUUUUAGU |
| | sequence, | 3′ |
| | artificial | |
| 5 | Partial gRNA | 5′ GGACCACCCCAAAAAUGAAGGGGACUAAAAC 3′ |
| | sequence, | |
| | artificial | |
| 6 | Partial gRNA | 5′ |
| | sequence, | GGACCACCCCAAAAAUGAAGGGGACUAAAACACAAA |
| | artificial | UCU 3′ |
| 7 | Target RNA | 5′ AGAUUUGUGUUUUAGU 3′ |
| | sequence, | |
| | artificial | |
| 8 | Partial gRNA | 5′ |
| | sequence, | GGCCACCCCAAAAAUGAAGGGGACUAAAACACAAAU |
| | artificial | XUAUCUGAAUAAAC 3′ |
| 9 | Target RNA | 5′ GUUUAUUCAGAUAZAUUUGUCACAGCAG 3′ |
| | sequence, | |
| | artificial | |
| 10 | Partial gRNA | 5′ |
| | sequence, | GGCCACCCCAAAAAUGAAGGGGACUAAAACAUAAGC |
| | artificial | CCAUCUAAAUAAAU 3′ |
| 11 | Target RNA | 5′ AUUUAUUUAGAUGGGCUUAUCACAGCAG 3′ |
| | sequence, | |
| | artificial | |
| 12 | Partial gRNA | 5′ |
| | sequence, | GGCCACCCCAAAAAUGAAGGGGACUAAAACGCAAAU |
| | artificial | CUAUCCGAGUGAGC 3′ |
| 13 | Target RNA | 5′ GCUCACUCGGAUAGAUUUGCCACAGCAG 3′ |
| | sequence, | |
| | artificial | |
| 14 | Wild-type | MKVTKVGGISHKKYTSEGRLVKSESEENRTDERLSALLN |
| | LbuCas13a | MRLDMYIKNPSSTETKENQKRIGKLKKFFSNKMVYLKD |
| | amino acid | NTLSLKNGKKENIDREYSETDILESDVRDKKNFAVLKKIY |
| | sequence, | LNENVNSEELEVFRNDIKKKLNKINSLKYSFEKNKANYQ |
| | Leptotrichia | KINENNIEKVEGKSKRNIIYDYYRESAKRDAYVSNVKEA |
| | buccalis | FDKLYKEEDIAKLVLEIENLTKLEKYKIREFYHEIIGRKND |
| | | KENFAKIIYEEIQNVNNMKELIEKVPDMSELKKSQVFYK |
| | | YYLDKEELNDKNIKYAFCHFVEIEMSQLLKNYVYKRLSN |
| | | ISNDKIKRIFEYQNLKKLIENKLLNKLDTYVRNCGKYNY |
| | | YLQDGEIATSDFIARNRQNEAFLRNIIGVSSVAYFSLRNIL |
| | | ETENENDITGRMRGKTVKNNKGEEKYVSGEVDKIYNEN |
| | | KKNEVKENLKMFYSYDFNMDNKNEIEDFFANIDEAISSIR |
| | | HGIVHFNLELEGKDIFAFKNIAPSEISKKMFQNEINEKKLK |
| | | LKIFRQLNSANVFRYLEKYKILNYLKRTRFEFVNKNIPFV |
| | | PSFTKLYSRIDDLKNSLGIYWKTPKTNDDNKTKEIIDAQI |
| | | YLLKNIYYGEFLNYFMSNNGNFFEISKEIIELNKNDKRNL |
| | | KTGFYKLQKFEDIQEKIPKEYLANIQSLYMINAGNQDEEE |
| | | KDTYIDFIQKIFLKGFMTYLANNGRLSLIYIGSDEETNTSL |
| | | AEKKQEFDKFLKKYEQNNNIKIPYEINEFLREIKLGNILKY |
| | | TERLNMFYLILKLLNHKELTNLKGSLEKYQSANKEEAFS |
| | | DQLELINLLNLDNNRVTEDFELEADEIGKFLDFNGNKVK |
| | | DNKELKKFDTNKIYFDGENIIKHRAFYNIKKYGMLNLLE |
| | | KIADKAGYKISIEELKKYSNKKNEIEKNHKMQENLHRKY |
| | | ARPRKDEKFTDEDYESYKQAIENIEEYTHLKNKVEFNEL |
| | | NLLQGLLLRILHRLVGYTSIWERDLRFRLKGEFPENQYIE |
| | | EIFNFENKKNVKYKGGQIVEKYIKFYKELHQNDEVKINK |
| | | YSSANIKVLKQEKKDLYIRNYIAHFNYIPHAEISLLEVLEN |
| | | LRKLLSYDRKLKNAVMKSVVDILKEYGFVATFKIGADK |
| | | KIGIQTLESEKIVHLKNLKKKKLMTDRNSEELCKLVKIMF |
| | | EYKMEEKKSEN |
| 15 | Variant | MKVTKVGGISHKKYTSEGRLVKSESEENRTDERLSALLN |
| | LbuCas13a | MRLDMYIKNPSSTETKENQKRIGKLKKFFSNKMVYLKD |
| | R337A, amino | NTLSLKNGKKENIDREYSETDILESDVRDKKNFAVLKKIY |
| | acid sequence, | LNENVNSEELEVFRNDIKKKLNKINSLKYSFEKNKANYQ |
| | artificial | KINENNIEKVEGKSKRNIIYDYYRESAKRDAYVSNVKEA |
| | | FDKLYKEEDIAKLVLEIENLTKLEKYKIREFYHEIIGRKND |
| | | KENFAKIIYEEIQNVNNMKELIEKVPDMSELKKSQVFYK |
| | | YYLDKEELNDKNIKYAFCHFVEIEMSQLLKNYVYKRLSN |
| | | ISNDKIKRIFEYQNLKKLIENKLLNKLDTYVRNCGKYNY |
| | | YLQDGEIATSDFIARNRQNEAFLANIIGVSSVAYFSLRNIL |
| | | ETENENDITGRMRGKTVKNNKGEEKYVSGEVDKIYNEN |
| | | KKNEVKENLKMFYSYDFNMDNKNEIEDFFANIDEAISSIR |
| | | HGIVHFNLELEGKDIFAFKNIAPSEISKKMFQNEINEKKLK |
| | | LKIFRQLNSANVFRYLEKYKILNYLKRTRFEFVNKNIPFV |
| | | PSFTKLYSRIDDLKNSLGIYWKTPKTNDDNKTKEIIDAQI |
| | | YLLKNIYYGEFLNYFMSNNGNFFEISKEIIELNKNDKRNL |
| | | KTGFYKLQKFEDIQEKIPKEYLANIQSLYMINAGNQDEEE |
| | | KDTYIDFIQKIFLKGFMTYLANNGRLSLIYIGSDEETNTSL |
| | | AEKKQEFDKFLKKYEQNNNIKIPYEINEFLREIKLGNILKY |
| | | TERLNMFYLILKLLNHKELTNLKGSLEKYQSANKEEAFS |
| | | DQLELINLLNLDNNRVTEDFELEADEIGKFLDFNGNKVK |
| | | DNKELKKFDTNKIYFDGENIIKHRAFYNIKKYGMLNLLE |
| | | KIADKAGYKISIEELKKYSNKKNEIEKNHKMQENLHRKY |
| | | ARPRKDEKFTDEDYESYKQAIENIEEYTHLKNKVEFNEL |
| | | NLLQGLLLRILHRLVGYTSIWERDLRFRLKGEFPENQYIE |
| | | EIFNFENKKNVKYKGGQIVEKYIKFYKELHQNDEVKINK |
| | | YSSANIKVLKQEKKDLYIRNYIAHFNYIPHAEISLLEVLEN |
| | | LRKLLSYDRKLKNAVMKSVVDILKEYGFVATFKIGADK |
| | | KIGIQTLESEKIVHLKNLKKKKLMTDRNSEELCKLVKIMF |
| | | EYKMEEKKSEN |
| 16 | Variant | MKVTKVGGISHKKYTSEGRLVKSESEENRTDERLSALLN |
| | LbuCas13a | MRLDMYIKNPSSTETKENQKRIGKLKKFFSNKMVYLKD |
| | N378A amino | NTLSLKNGKKENIDREYSETDILESDVRDKKNFAVLKKIY |
| | acid sequence, | LNENVNSEELEVFRNDIKKKLNKINSLKYSFEKNKANYQ |
| | artificial | KINENNIEKVEGKSKRNIIYDYYRESAKRDAYVSNVKEA |
| | | FDKLYKEEDIAKLVLEIENLTKLEKYKIREFYHEIIGRKND |
| | | KENFAKIIYEEIQNVNNMKELIEKVPDMSELKKSQVFYK |
| | | YYLDKEELNDKNIKYAFCHFVEIEMSQLLKNYVYKRLSN |
| | | ISNDKIKRIFEYQNLKKLIENKLLNKLDTYVRNCGKYNY |
| | | YLQDGEIATSDFIARNRQNEAFLRAIIGVSSVAYFSLRNIL |
| | | ETENENDITGRMRGKTVKNNKGEEKYVSGEVDKIYNEN |
| | | KKNEVKENLKMFYSYDFNMDNKNEIEDFFANIDEAISSIR |
| | | HGIVHFNLELEGKDIFAFKNIAPSEISKKMFQNEINEKKLK |
| | | LKIFRQLNSANVFRYLEKYKILNYLKRTRFEFVNKNIPFV |
| | | PSFTKLYSRIDDLKNSLGIYWKTPKTNDDNKTKEIIDAQI |
| | | YLLKNIYYGEFLNYFMSNNGNFFEISKEIIELNKNDKRNL |
| | | KTGFYKLQKFEDIQEKIPKEYLANIQSLYMINAGNQDEEE |
| | | KDTYIDFIQKIFLKGFMTYLANNGRLSLIYIGSDEETNTSL |
| | | AEKKQEFDKFLKKYEQNNNIKIPYEINEFLREIKLGNILKY |
| | | TERLNMFYLILKLLNHKELTNLKGSLEKYQSANKEEAFS |
| | | DQLELINLLNLDNNRVTEDFELEADEIGKFLDFNGNKVK |
| | | DNKELKKFDTNKIYFDGENIIKHRAFYNIKKYGMLNLLE |
| | | KIADKAGYKISIEELKKYSNKKNEIEKNHKMQENLHRKY |
| | | ARPRKDEKFTDEDYESYKQAIENIEEYTHLKNKVEFNEL |
| | | NLLQGLLLRILHRLVGYTSIWERDLRFRLKGEFPENQYIE |
| | | EIFNFENKKNVKYKGGQIVEKYIKFYKELHQNDEVKINK |
| | | YSSANIKVLKQEKKDLYIRNYIAHFNYIPHAEISLLEVLEN |
| | | LRKLLSYDRKLKNAVMKSVVDILKEYGFVATFKIGADK |
| | | KIGIQTLESEKIVHLKNLKKKKLMTDRNSEELCKLVKIMF |
| | | EYKMEEKKSEN |
| 17 | Variant | MKVTKVGGISHKKYTSEGRLVKSESEENRTDERLSALLN |
| | LbuCas13a | MRLDMYIKNPSSTETKENQKRIGKLKKFFSNKMVYLKD |
| | R963A amino | NTLSLKNGKKENIDREYSETDILESDVRDKKNFAVLKKIY |
| | acid sequence, | LNENVNSEELEVFRNDIKKKLNKINSLKYSFEKNKANYQ |
| | artificial | KINENNIEKVEGKSKRNIIYDYYRESAKRDAYVSNVKEA |
| | | FDKLYKEEDIAKLVLEIENLTKLEKYKIREFYHEIIGRKND |
| | | KENFAKIIYEEIQNVNNMKELIEKVPDMSELKKSQVFYK |
| | | YYLDKEELNDKNIKYAFCHFVEIEMSQLLKNYVYKRLSN |
| | | ISNDKIKRIFEYQNLKKLIENKLLNKLDTYVRNCGKYNY |
| | | YLQDGEIATSDFIARNRQNEAFLRNIIGVSSVAYFSLRNIL |
| | | ETENENDITGRMRGKTVKNNKGEEKYVSGEVDKIYNEN |
| | | KKNEVKENLKMFYSYDFNMDNKNEIEDFFANIDEAISSIR |
| | | HGIVHFNLELEGKDIFAFKNIAPSEISKKMFQNEINEKKLK |
| | | LKIFRQLNSANVFRYLEKYKILNYLKRTRFEFVNKNIPFV |
| | | PSFTKLYSRIDDLKNSLGIYWKTPKTNDDNKTKEIIDAQI |
| | | YLLKNIYYGEFLNYFMSNNGNFFEISKEIIELNKNDKRNL |
| | | KTGFYKLQKFEDIQEKIPKEYLANIQSLYMINAGNQDEEE |
| | | KDTYIDFIQKIFLKGFMTYLANNGRLSLIYIGSDEETNTSL |
| | | AEKKQEFDKFLKKYEQNNNIKIPYEINEFLREIKLGNILKY |
| | | TERLNMFYLILKLLNHKELTNLKGSLEKYQSANKEEAFS |
| | | DQLELINLLNLDNNRVTEDFELEADEIGKFLDFNGNKVK |
| | | DNKELKKFDTNKIYFDGENIIKHRAFYNIKKYGMLNLLE |
| | | KIADKAGYKISIEELKKYSNKKNEIEKNHKMQENLHRKY |
| | | ARPRKDEKFTDEDYESYKQAIENIEEYTHLKNKVEFNEL |
| | | NLLQGLLLRILHALVGYTSIWERDLRFRLKGEFPENQYIE |
| | | EIFNFENKKNVKYKGGQIVEKYIKFYKELHQNDEVKINK |
| | | YSSANIKVLKQEKKDLYIRNYIAHFNYIPHAEISLLEVLEN |
| | | LRKLLSYDRKLKNAVMKSVVDILKEYGFVATFKIGADK |
| | | KIGIQTLESEKIVHLKNLKKKKLMTDRNSEELCKLVKIMF |
| | | EYKMEEKKSEN |
| 18 | Variant | MKVTKVGGISHKKYTSEGRLVKSESEENRTDERLSALLN |
| | LbuCas13a | MRLDMYIKNPSSTETKENQKRIGKLKKFFSNKMVYLKD |
| | R973A amino | NTLSLKNGKKENIDREYSETDILESDVRDKKNFAVLKKIY |
| | acid sequence, | LNENVNSEELEVFRNDIKKKLNKINSLKYSFEKNKANYQ |
| | artificial | KINENNIEKVEGKSKRNIIYDYYRESAKRDAYVSNVKEA |
| | | FDKLYKEEDIAKLVLEIENLTKLEKYKIREFYHEIIGRKND |
| | | KENFAKIIYEEIQNVNNMKELIEKVPDMSELKKSQVFYK |
| | | YYLDKEELNDKNIKYAFCHFVEIEMSQLLKNYVYKRLSN |
| | | ISNDKIKRIFEYQNLKKLIENKLLNKLDTYVRNCGKYNY |
| | | YLQDGEIATSDFIARNRQNEAFLRNIIGVSSVAYFSLRNIL |
| | | ETENENDITGRMRGKTVKNNKGEEKYVSGEVDKIYNEN |
| | | KKNEVKENLKMFYSYDFNMDNKNEIEDFFANIDEAISSIR |
| | | HGIVHFNLELEGKDIFAFKNIAPSEISKKMFQNEINEKKLK |
| | | LKIFRQLNSANVFRYLEKYKILNYLKRTRFEFVNKNIPFV |
| | | PSFTKLYSRIDDLKNSLGIYWKTPKTNDDNKTKEIIDAQI |
| | | YLLKNIYYGEFLNYFMSNNGNFFEISKEIIELNKNDKRNL |
| | | KTGFYKLQKFEDIQEKIPKEYLANIQSLYMINAGNQDEEE |
| | | KDTYIDFIQKIFLKGFMTYLANNGRLSLIYIGSDEETNTSL |
| | | AEKKQEFDKFLKKYEQNNNIKIPYEINEFLREIKLGNILKY |
| | | TERLNMFYLILKLLNHKELTNLKGSLEKYQSANKEEAFS |
| | | DQLELINLLNLDNNRVTEDFELEADEIGKFLDFNGNKVK |
| | | DNKELKKFDTNKIYFDGENIIKHRAFYNIKKYGMLNLLE |
| | | KIADKAGYKISIEELKKYSNKKNEIEKNHKMQENLHRKY |
| | | ARPRKDEKFTDEDYESYKQAIENIEEYTHLKNKVEFNEL |
| | | NLLQGLLLRILHRLVGYTSIWEADLRFRLKGEFPENQYIE |
| | | EIFNFENKKNVKYKGGQIVEKYIKFYKELHQNDEVKINK |
| | | YSSANIKVLKQEKKDLYIRNYIAHFNYIPHAEISLLEVLEN |
| | | LRKLLSYDRKLKNAVMKSVVDILKEYGFVATFKIGADK |
| | | KIGIQTLESEKIVHLKNLKKKKLMTDRNSEELCKLVKIMF |
| | | EYKMEEKKSEN |
| 19 | Wild-type | GGACCACCCCAAAAAUGAAGGGGACUAAAAC |
| | sequence of the | |
| | handle region | |
| | of LbuCas13a | |
| | guide RNA, | |
| | synthetic | |
| 20 | Mutated | GGCCACCCCAAAAAUGAAGGGGACUAAAAC |
| | sequence of the | |
| | handle region | |
| | of LbuCas13a | |
| | guide RNA, | |
| | synthetic | |
| 21 | AM78_Lbu_ | AAGCGTTTCTTGCCAACATCATTGGGGT |
| | R377A_ATH_fwd | |
| 22 | AM79_Lbu_ | AAGCGTTTCTTCGCGCCATCATTGGGGT |
| | N378A_ATH_ | |
| | fwd | |
| 23 | AM80_Lbu_ | CATTCTGACGGTTGCGGGCGAT |
| | 377_378_ATH_ | |
| | reV | |
| 24 | AM89_Lbu_ | GAAGCGCAGATCGGCTTCCCAAATTG |
| | R973A_ATH_rev | |
| 25 | AM90_Lbu_ | CGCCTTAAAGGTGAGTTCCCAGAAAACCAAT |
| | R973A_ATH_fwd | |
| 26 | AM85_Lbu_ | AGGACTATGAAAGTTACAAGCAAGCT |
| | 973_seq_fwd | |
| 27 | AM86_Lbu_ | TTGACACGTACGTCCGTAATTGT |
| | 377_378_seq_fwd | |
| 28 | MOC- | GGCGTAATACGACTCACTATAGG |
| | 626_T7_opt | |
| 29 | MOC- | GTGTGGGCTTCTGCTGTGACAAATCTATCTGAATAAAC |
| | 1226_longer_ | TCTTCTTCTTGGTTTCCCtatagtgagtcgtattacgcc |
| | AM_Liu_Lbu_ | |
| | target_IVTtemp | |
| 30 | MOC- | GTGTGGGCTTACTAAAACACAAATCTATCTGAATAAA |
| | 1227_longer_ | CTCTTCTTCTTGGTTTCCCtatagtgagtcgtattacgcc |
| | AM_Liu_Lbu_ | |
| | antitag_IVTtemp | |
| 31 | AM91_Lbu_ | GTGTGGGCTTCTGCTGTGG |
| | Liu_target_ | ACAAATCTATCTGAATAAACTCTTGAAG |
| | MM25:28 | TTGGTTTCCCtatagtgagtcgtattacgcc |
| 32 | AM92_Lbu_ | GTGTGGGCTTCTGCTGTGG |
| | Liu_target_ | ACAAATCTATCTGAATAAACAGAACTTC |
| | MM21:24 | TTGGTTTCCCtatagtgagtcgtattacgcc |
| 33 | AM93_Lbu_ | GTGTGGGCTTCTGCTGTGG |
| | Liu_target_ | ACAAATCTATCTGAATTTTGTCTTCTTC |
| | MM17:20 | TTGGTTTCCCtatagtgagtcgtattacgcc |
| 34 | AM94_Lbu_ | GTGTGGGCTTCTGCTGTGG |
| | Liu_target_ | ACAAATCTATCTCTTAAAACTCTTCTTC |
| | MM13:16 | TTGGTTTCCCtatagtgagtcgtattacgcc |
| 35 | AM95_Lbu_ | GTGTGGGCTTCTGCTGTGG |
| | Liu_target_ | ACAAATCTTAGAGAATAAACTCTTCTTC |
| | MM9:12 | TTGGTTTCCCtatagtgagtcgtattacgcc |
| 36 | AM96_Lbu_ | GTGTGGGCTTCTGCTGTGG |
| | Liu_target_ | ACAATAGAATCTGAATAAACTCTTCTTC |
| | MM5:8 | TTGGTTTCCCtatagtgagtcgtattacgcc |
| 37 | AM97_Lbu_ | GTGTGGGCTTCTGCTGTGG |
| | Liu_target_ | TGTTATCTATCTGAATAAACTCTTCTTC |
| | MM1:4 | TTGGTTTCCCtatagtgagtcgtattacgcc |
| 38 | AM108_Lbu_ | GTGTGGGCTTCTGCTGTGG |
| | Liu_target_SM1 | tCAAATCTATCTGAATAAACTCTTCTTC |
| | | TTGGTTTCCCtatagtgagtcgtattacgcc |
| 39 | AM109_Lbu_ | GTGTGGGCTTCTGCTGTGG |
| | Liu_target_SM2 | AgAAATCTATCTGAATAAACTCTTCTTC |
| | | TTGGTTTCCCtatagtgagtcgtattacgcc |
| 40 | AM110_Lbu_ | GTGTGGGCTTCTGCTGTGG |
| | Liu_target_SM3 | ACtAATCTATCTGAATAAACTCTTCTTC |
| | | TTGGTTTCCCtatagtgagtcgtattacgcc |
| 41 | AM111_Lbu_ | GTGTGGGCTTCTGCTGTGG |
| | Liu_target_SM4 | ACAtATCTATCTGAATAAACTCTTCTTC |
| | | TTGGTTTCCCtatagtgagtcgtattacgcc |
| 42 | AM112_Lbu_ | GTGTGGGCTTCTGCTGTGG |
| | Liu_target_SM5 | ACAAtTCTATCTGAATAAACTCTTCTTC |
| | | TTGGTTTCCCtatagtgagtcgtattacgcc |
| 43 | AM113_Lbu_ | GTGTGGGCTTCTGCTGTGG |
| | Liu_target_SM6 | ACAAAaCTATCTGAATAAACTCTTCTTC |
| | | TTGGTTTCCCtatagtgagtcgtattacgcc |
| 44 | AM114_Lbu_ | GTGTGGGCTTCTGCTGTGG |
| | Liu_target_SM7 | ACAAATgTATCTGAATAAACTCTTCTTC |
| | | TTGGTTTCCCtatagtgagtcgtattacgcc |
| 45 | AM115_Lbu_ | GTGTGGGCTTCTGCTGTGG |
| | Liu_target_SM8 | ACAAATCaATCTGAATAAACTCTTCTTC |
| | | TTGGTTTCCCtatagtgagtcgtattacgcc |
| 46 | AM116_Lbu_ | GTGTGGGCTTCTGCTGTGG |
| | Liu_target_SM9 | ACAAATCTtTCTGAATAAACTCTTCTTC |
| | | TTGGTTTCCCtatagtgagtcgtattacgcc |
| 47 | AM117_Lbu_ | GTGTGGGCTTCTGCTGTGG |
| | Liu_target_ | ACAAATCTAaCTGAATAAACTCTTCTTC |
| | SM10 | TTGGTTTCCCtatagtgagtcgtattacgcc |
| 48 | AM118_Lbu_ | GTGTGGGCTTCTGCTGTGG |
| | Liu_target_ | ACAAATCTATgTGAATAAACTCTTCTTC |
| | SM11 | TTGGTTTCCCtatagtgagtcgtattacgcc |
| 49 | AM119_Lbu_ | GTGTGGGCTTCTGCTGTGG |
| | Liu_target_ | ACAAATCTATCaGAATAAACTCTTCTTC |
| | SM12 | TTGGTTTCCCtatagtgagtcgtattacgcc |
| 50 | AM120_Lbu_ | GTGTGGGCTTCTGCTGTGG |
| | Liu_target_ | ACAAATCTATCTcAATAAACTCTTCTTC |
| | SM13 | TTGGTTTCCCtatagtgagtcgtattacgcc |
| 51 | AM121_Lbu_ | GTGTGGGCTTCTGCTGTGG |
| | Liu_target_ | ACAAATCTATCTGtATAAACTCTTCTTC |
| | SM14 | TTGGTTTCCCtatagtgagtcgtattacgcc |
| 52 | AM122_Lbu_ | GTGTGGGCTTCTGCTGTGG |
| | Liu_target_ | ACAAATCTATCTGAtTAAACTCTTCTTC |
| | SM15 | TTGGTTTCCCtatagtgagtcgtattacgcc |
| 53 | AM123_Lbu_ | GTGTGGGCTTCTGCTGTGG |
| | Liu_target_ | ACAAATCTATCTGAAaAAACTCTTCTTC |
| | SM16 | TTGGTTTCCCtatagtgagtcgtattacgcc |
| 54 | AM124_Lbu_ | GTGTGGGCTTCTGCTGTGG |
| | Liu_target_ | ACAAATCTATCTGAATtAACTCTTCTTC |
| | SM17 | TTGGTTTCCCtatagtgagtcgtattacgcc |
| 55 | AM125_Lbu_ | GTGTGGGCTTCTGCTGTGG |
| | Liu_target_ | ACAAATCTATCTGAATAtACTCTTCTTC |
| | SM18 | TTGGTTTCCCtatagtgagtegtattacgcc |
| 56 | AM126_Lbu_ | GTGTGGGCTTCTGCTGTGG |
| | Liu_target_ | ACAAATCTATCTGAATAAtCTCTTCTTC |
| | SM19 | TTGGTTTCCCtatagtgagtcgtattacgcc |
| 57 | AM127_Lbu_ | GTGTGGGCTTCTGCTGTGG |
| | Liu_target_ | ACAAATCTATCTGAATAAAgTCTTCTTC |
| | SM20 | TTGGTTTCCCtatagtgagtcgtattacgcc |
| 58 | AM160_SARS_ | CATTAAATGGTAGGACAGGGTTATCAAACCTCTTAGTA |
| | COV2_S_D80_ | CCATTGGTCCCAGAGACCCtatagtgagtcgtattacgcc |
| | WT | |
| 59 | AM161_SARS_ | CATTAAATGGTAGGACAGGGTTAGCAAACCTCTTAGT |
| | COV2_S_D80_ | ACCATTGGTCCCAGAGACCCtatagtgagtcgtattacgcc |
| | BETA | |
| 60 | AM164_SARS_ | TTAGACTTCCTAAACAATCTATACAGGTAATTATAATT |
| | COV2_S_ | ACCACCAACCTTAGAATCCtatagtgagtcgtattacgcc |
| | L452_WT | |
| 61 | AM165_SARS_ | TTAGACTTCCTAAACAATCTATACCGGTAATTATAATT |
| | COV2_S_ | ACCACCAACCTTAGAATCCtatagtgagtcgtattacgcc |
| | L452_DELTA | |
| 62 | AM168_SARS_ | AAACCTTCAACACCATTACAAGGTGTGCTACCGGCCTG |
| | COV2_S_ | ATAGATTTCAGTTGAAACCtatagtgagtegtattacgcc |
| | S477_WT | |
| 63 | AM169_SARS_ | AAACCTTCAACACCATTACAAGGTTTGTTACCGGCCTG |
| | COV2_S_ | ATAGATTTCAGTTGAAACCtatagtgagtcgtattacgcc |
| | S477_OMICRON | |
| 64 | MOC- | GGACCACCCCAAAAAUGAAGGGGACUAAAACACAAA |
| | 1264_Lbu_mat_ | UCUAUCUGAAUAAACUCUUCUUC |
| | crRNA_Liu_ | |
| | 28 nt | |
| 65 | MOC- | GGACCACCCCAAAAAUGAAGGGGACUAAAACACAAA |
| | 1265_Lbu_mat_ | UCUAUCUGAAUAAAC |
| | crRNA_Liu_ | |
| | 20 nt | |
| 66 | MOC- | GGACCACCCCAAAAAUGAAGGGGACUAAAACACAAA |
| | 1266_Lbu_mat_ | UCUAUCUGAAU |
| | crRNA_Liu_ | |
| | 16 nt | |
| 67 | AM208_Liu_ | GGCCACCCCAAAAAUGAAGGGGACUAAAACACAAAU |
| | mut_DR_28 | CUAUCUGAAUAAACUCUUCUUC |
| 68 | AM211_Liu_ | GGCCACCCCAAAAAUGAAGGGGACUAAAACACAAAU |
| | mut_DR_Lbu_20 | CUAUCUGAAUAAAC |
| 69 | MOC- | GGGAAACCAAGAAGAAGAGTTTATTCAGATAGATTTG |
| | 1284_Liu_long_ | TCACAGCAGAAGCCCACAC |
| | target_IDT_ | |
| | RNA | |
| 70 | MOC- | GGGAAACCAAGAAGAAGAGTTTATTCAGATAGATTTG |
| | 1285_Liu_long_ | TGTTTTAGTAAGCCCACAC |
| | anti- | |
| | target_IDT_ | |
| | RNA | |
| 71 | Liu_Perfect_ | GGGAAACCAA |
| | match_target_ | GAAGAAGAGUUUAUUCAGAUAGAUUUGU |
| | RNA | CACAGCAGAAGCCCACAC |
| 72 | Liu_target_ | GGGAAACCAA |
| | RNA_MM1-4 | GAAGAAGAGUUUAUUCAGAUAGAUAACA |
| | | CACAGCAGAAGCCCACAC |
| 73 | Liu_target_ | GGGAAACCAA |
| | RNA_MM5-8 | GAAGAAGAGUUUAUUCAGAUUCUAUUGU |
| | | CACAGCAGAAGCCCACAC |
| 74 | Liu_target_ | GGGAAACCAA |
| | RNA_MM9-12 | GAAGAAGAGUUUAUUCUCUAAGAUUUGU |
| | | CACAGCAGAAGCCCACAC |
| 75 | Liu_target_ | GGGAAACCAA |
| | RNA_MM13-16 | GAAGAAGAGUUUUAAGAGAUAGAUUUGU |
| | | CACAGCAGAAGCCCACAC |
| 76 | Liu_target_ | GGGAAACCAA |
| | RNA_MM17-20 | GAAGAAGACAAAAUUCAGAUAGAUUUGU |
| | | CACAGCAGAAGCCCACAC |
| 77 | Liu_target_ | GGGAAACCAA |
| | RNA_MM21-24 | GAAGUUCUGUUUAUUCAGAUAGAUUUGU |
| | | CACAGCAGAAGCCCACAC |
| 78 | Liu_target_ | GGGAAACCAA |
| | RNA_MM25-28 | CUUCAAGAGUUUAUUCAGAUAGAUUUGU |
| | | CACAGCAGAAGCCCACAC |
| 79 | Liu_target_ | GGGAAACCAA |
| | RNA_SM1 | GAAGAAGAGUUUAUUCAGAUAGAUUUGa |
| | | CACAGCAGAAGCCCACAC |
| 80 | Liu_target_ | GGGAAACCAA |
| | RNA_SM2 | GAAGAAGAGUUUAUUCAGAUAGAUUUCU |
| | | CACAGCAGAAGCCCACAC |
| 81 | Liu_target_ | GGGAAACCAA |
| | RNA_SM3 | GAAGAAGAGUUUAUUCAGAUAGAUUaGU |
| | | CACAGCAGAAGCCCACAC |
| 82 | Liu_target_ | GGGAAACCAA |
| | RNA_SM4 | GAAGAAGAGUUUAUUCAGAUAGAUaUGU |
| | | CACAGCAGAAGCCCACAC |
| 83 | Liu_target_ | GGGAAACCAA |
| | RNA_SM5 | GAAGAAGAGUUUAUUCAGAUAGAaUUGU |
| | | CACAGCAGAAGCCCACAC |
| 84 | Liu_target_ | GGGAAACCAA |
| | RNA_SM6 | GAAGAAGAGUUUAUUCAGAUAGUUUUGU |
| | | CACAGCAGAAGCCCACAC |
| 85 | Liu_target_ | GGGAAACCAA |
| | RNA_SM7 | GAAGAAGAGUUUAUUCAGAUAcAUUUGU |
| | | CACAGCAGAAGCCCACAC |
| 86 | Liu_target_ | GGGAAACCAA |
| | RNA_SM8 | GAAGAAGAGUUUAUUCAGAUUGAUUUGU |
| | | CACAGCAGAAGCCCACAC |
| 87 | Liu_target_ | GGGAAACCAA |
| | RNA_SM9 | GAAGAAGAGUUUAUUCAGAaAGAUUUGU |
| | | CACAGCAGAAGCCCACAC |
| 88 | Liu_target_ | GGGAAACCAA |
| | RNA_SM10 | GAAGAAGAGUUUAUUCAGUUAGAUUUGU |
| | | CACAGCAGAAGCCCACAC |
| 89 | Liu_target_ | GGGAAACCAA |
| | RNA_SM11 | GAAGAAGAGUUUAUUCACAUAGAUUUGU |
| | | CACAGCAGAAGCCCACAC |
| 90 | Liu_target_ | GGGAAACCAA |
| | RNA_SM12 | GAAGAAGAGUUUAUUCUGAUAGAUUUGU |
| | | CACAGCAGAAGCCCACAC |
| 91 | Liu_target_ | GGGAAACCAA |
| | RNA_SM13 | GAAGAAGAGUUUAUUgAGAUAGAUUUGU |
| | | CACAGCAGAAGCCCACAC |
| 92 | Liu_target_ | GGGAAACCAA |
| | RNA_SM14 | GAAGAAGAGUUUAUaCAGAUAGAUUUGU |
| | | CACAGCAGAAGCCCACAC |
| 93 | Liu_target_ | GGGAAACCAA |
| | RNA_SM15 | GAAGAAGAGUUUAaUCAGAUAGAUUUGU |
| | | CACAGCAGAAGCCCACAC |
| 94 | Liu_target_ | GGGAAACCAA |
| | RNA_SM16 | GAAGAAGAGUUUUUUCAGAUAGAUUUGU |
| | | CACAGCAGAAGCCCACAC |
| 95 | Liu_target_ | GGGAAACCAA |
| | RNA_SM17 | GAAGAAGAGUUaAUUCAGAUAGAUUUGU |
| | | CACAGCAGAAGCCCACAC |
| 96 | Liu_target_ | GGGAAACCAA |
| | RNA_SM18 | GAAGAAGAGUaUAUUCAGAUAGAUUUGU |
| | | CACAGCAGAAGCCCACAC |
| 97 | Liu_target_ | GGGAAACCAA |
| | RNA_SM19 | GAAGAAGAGaUUAUUCAGAUAGAUUUGU |
| | | CACAGCAGAAGCCCACAC |
| 98 | Liu_target_ | GGGAAACCAA |
| | RNA_SM20 | GAAGAAGAcUUUAUUCAGAUAGAUUUGU |
| | | CACAGCAGAAGCCCACAC |
| 99 | AM160_SARS_ | GGGTCTCTGGGACCAATGGTACTAAGAGGTTTGATAA |
| | COV2_S_D80_ | CCCTGTCCTACCATTTAATG |
| | WT | |
| 100 | AM161_SARS_ | GGGTCTCTGGGACCAATGGTACTAAGAGGTTTGCTAA |
| | COV2_S_D80_ | CCCTGTCCTACCATTTAATG |
| | BETA | |
| 101 | AM164_SARS_ | GGATTCTAAGGTTGGTGGTAATTATAATTACCTGTATA |
| | COV2_S_ | GATTGTTTAGGAAGTCTAA |
| | L452_WT | |
| 102 | AM165_SARS_ | GGATTCTAAGGTTGGTGGTAATTATAATTACCGGTATA |
| | COV2_S_ | GATTGTTTAGGAAGTCTAA |
| | L452_DELTA | |
| 103 | AM168_SARS_ | GGTTTCAACTGAAATCTATCAGGCCGGTAGCACACCTT |
| | COV2_S_ | GTAATGGTGTTGAAGGTTT |
| | S477_WT | |
| 104 | AM169_SARS_ | GGTTTCAACTGAAATCTATCAGGCCGGTAACAAACCTT |
| | COV2_S_ | GTAATGGTGTTGAAGGTTT |
| | S477_OMICRON | |
| 105 | AM162_WT_ | GGACCACCCCAAAAAUGAAGGGGACUAAAACGGGUU |
| | crRNA_S_D80_ | AUCAAACCUCUUAGU |
| | nt | |
| 106 | AM163_beta_ | GGACCACCCCAAAAAUGAAGGGGACUAAAACGGGUU |
| | crRNA_S_D80_ | AGCAAACCUCUUAGU |
| | 20 nt | |
| 107 | AM166_WT_ | GGACCACCCCAAAAAUGAAGGGGACUAAAACCUAUA |
| | crRNA_L452_S_ | CAGGUAAUUAUAAUU |
| | 20 nt | |
| 108 | AM167_Delta_ | GGACCACCCCAAAAAUGAAGGGGACUAAAACCUAUA |
| | crRNA_L452R_ | CCGGUAAUUAUAAUU |
| | S_20 nt | |
| 109 | AM170_WT_ | GGACCACCCCAAAAAUGAAGGGGACUAAAACCAAGG |
| | crRNA_S477_S_ | UGUGCUACCGGCCUG |
| | 20 nt | |
| 110 | AM171_omicron_ | GGACCACCCCAAAAAUGAAGGGGACUAAAACCAAGG |
| | crRNA_477_ | UUUGUUACCGGCCUG |
| | 478_S_20 nt | |
| 111 | AM194_WT_ | GGACCACCCCAAAAAUGAAGGGGACUAAAACGGGUU |
| | crRNA_S_D80_ | AUCAAACCUCUUACU |
| | 20 nt_MM19 | |
| 112 | AM195_beta_ | GGACCACCCCAAAAAUGAAGGGGACUAAAACGGGUU |
| | crRNA_S_D80_ | AGCAAACCUCUUACU |
| | 20 nt_MM7_19 | |
| 113 | AM196_WT_ | GGACCACCCCAAAAAUGAAGGGGACUAAAACCUAUA |
| | crRNA_L452_S_ | CAGGUAAUUAUAAAU |
| | 20 nt_MM19 | |
| 114 | AM197_Delta_ | GGACCACCCCAAAAAUGAAGGGGACUAAAACCUAUA |
| | crRNA_L452R_ | CCGGUAAUUAUAAAU |
| | S_20 nt_MM7_ | |
| | 19 | |
| 115 | AM198_WT_ | GGACCACCCCAAAAAUGAAGGGGACUAAAACCAAGG |
| | crRNA_S477_S_ | UGUGCUACCGGCCAG |
| | 20 nt_MM19 | |
| 116 | AM199_omicron_ | GGACCACCCCAAAAAUGAAGGGGACUAAAACCAAGG |
| | crRNA_477_ | UUUGUUACCGGCCAG |
| | 478_S_20 nt_ | |
| | MM7_19 | |
| 117 | AM202_wt_ | GGACCACCCCAAAAAUGAAGGGGACUAAAACAAUGG |
| | crRNA_D80_v2 | UAGGACAGGGUUAUC |
| 118 | AM203_beta_ | GGACCACCCCAAAAAUGAAGGGGACUAAAACAAUGG |
| | crRNA_D80_v2_ | UAGGACAGGGUUAGC |
| | MM19 | |
| 119 | AM204_wt_ | GGACCACCCCAAAAAUGAAGGGGACUAAAACUUCCU |
| | crRNA_L452_v2 | AAACAAUCUAUACAG |
| 120 | AM205_delta_ | GGACCACCCCAAAAAUGAAGGGGACUAAAACUUCCU |
| | crRNA_L452_ | AAACAAUCUAUACCG |
| | v2_MM19 | |
| 121 | AM206_wt_ | GGACCACCCCAAAAAUGAAGGGGACUAAAACACACC |
| | crRNA_S477_v2 | AUUACAAGGUGUGCU |
| 122 | AM207_omicron_ | GGACCACCCCAAAAAUGAAGGGGACUAAAACACACC |
| | crRNA_S477_ | AUUACAAGGUUUGUU |
| | v2_MM19 | |
| 123 | AM212_WT_ | GGCCACCCCAAAAAUGAAGGGGACUAAAACGGGUUA |
| | del_crRNA_S_ | UCAAACCUCUUAGU |
| | D80_nt | |
| 124 | AM213_beta_ | GGCCACCCCAAAAAUGAAGGGGACUAAAACGGGUUA |
| | del_crRNA_S_ | GCAAACCUCUUAGU |
| | D80_20 nt | |
| 125 | AM214_WT_ | GGCCACCCCAAAAAUGAAGGGGACUAAAACCUAUAC |
| | del_crRNA_ | AGGUAAUUAUAAUU |
| | L452_S_20 nt | |
| 126 | AM215_Delta_ | GGCCACCCCAAAAAUGAAGGGGACUAAAACCUAUAC |
| | del_crRNA_ | CGGUAAUUAUAAUU |
| | L452R_S_20 nt | |
| 127 | AM216_WT_ | GGCCACCCCAAAAAUGAAGGGGACUAAAACCAAGGU |
| | del_crRNA_ | GUGCUACCGGCCUG |
| | S477_S_20 nt | |
| 128 | AM217_omicron_ | GGCCACCCCAAAAAUGAAGGGGACUAAAACCAAGGU |
| | del_crRNA_ | UUGUUACCGGCCUG |
| | 477_478_S_ | |
| | 20 nt | |
| 129 | AM222_crRNA_ | GGCCACCCCAAAAAUGAAGGGGACUAAAACGGGUUA |
| | S_D80_20 nt_ | UCAAACCUCUUACU |
| | MM19_mut_ | |
| | DR | |
| 130 | AM223_beta_ | GGCCACCCCAAAAAUGAAGGGGACUAAAACGGGUUA |
| | crRNA_S_D80_ | GCAAACCUCUUACU |
| | 20 nt_MM7_19_ | |
| | mut_DR | |
| 131 | AM224_WT_ | GGCCACCCCAAAAAUGAAGGGGACUAAAACCUAUAC |
| | crRNA_L452_S_ | AGGUAAUUAUAAAU |
| | 20 nt_MM19_ | |
| | mut_DR | |
| 132 | AM225_Delta_ | GGCCACCCCAAAAAUGAAGGGGACUAAAACCUAUAC |
| | crRNA_L452R_ | CGGUAAUUAUAAAU |
| | S_20 nt_MM7_ | |
| | 19_mut_DR | |
| 133 | AM226_WT_ | GGCCACCCCAAAAAUGAAGGGGACUAAAACCAAGGU |
| | crRNA_S477_S_ | GUGCUACCGGCCAG |
| | 20 nt_MM19_ | |
| | mut_DR | |
| 134 | AM227_omicron_ | GGCCACCCCAAAAAUGAAGGGGACUAAAACCAAGGU |
| | crRNA_477_ | UUGUUACCGGCCAG |
| | 478_S_20 nt_ | |
| | MM7_19_mut_ | |
| | DR | |
| 135 | AM228_Liu_ | GGCCACCCCAAAAAUGAAGGGGACUAAAACACAAAU |
| | mut_DR_Lbu_20_ | GUAUCUGAAUAAAC |
| | pos7_G | |
| 136 | AM229_Liu_ | GGCCACCCCAAAAAUGAAGGGGACUAAAACACAAAU |
| | mut_DR_Lbu_20_ | AUAUCUGAAUAAAC |
| | pos7_A | |
| 137 | AM230_Liu_ | GGCCACCCCAAAAAUGAAGGGGACUAAAACACAAAU |
| | mut_DR_Lbu_20_ | UUAUCUGAAUAAAC |
| | pos7_U | |
| 138 | AM243_WT_ | GGCCACCCCAAAAAUGAAGGGGACUAAAACGGGUUU |
| | del_crRNA_S_ | UCAAACCUCUUAGU |
| | D80_20 nt_MM6 | |
| 139 | AM244_beta_ | GGCCACCCCAAAAAUGAAGGGGACUAAAACGGGUUU |
| | del_crRNA_S_ | GCAAACCUCUUAGU |
| | D80_20 nt_MM6 | |
| 140 | AM245_WT_ | GGCCACCCCAAAAAUGAAGGGGACUAAAACCUAUAG |
| | del_crRNA_ | AGGUAAUUAUAAUU |
| | L452_S_20 nt_ | |
| | MM6 | |
| 141 | AM246_Delta_ | GGCCACCCCAAAAAUGAAGGGGACUAAAACCUAUAG |
| | del_crRNA_ | CGGUAAUUAUAAUU |
| | L452R_S_20 nt_ | |
| | MM6 | |
| 142 | AM247_WT_ | GGCCACCCCAAAAAUGAAGGGGACUAAAACCAAGGA |
| | del_crRNA_ | GUGCUACCGGCCUG |
| | S477_S_20 nt_ | |
| | MM6 | |
| 143 | AM248_omicron_ | GGCCACCCCAAAAAUGAAGGGGACUAAAACCAAGGA |
| | del_crRNA_ | UUGUUACCGGCCUG |
| | 477_478_S_ | |
| | 20 nt_MM6 | |
| 144 | AM249_WT_ | GGACCACCCCAAAAAUGAAGGGGACUAAAACGGGUU |
| | crRNA_S_D80_ | UUCAAACCUCUUACU |
| | 20 nt_MM6_19 | |
| 145 | Liu_modified_ | GGGAAACCAA |
| | localGC_3_G | GAAUAAGAGCUCACUCGGAUACAUUUGC |
| | | CACAGCAGAAGCCCACAC |
| 146 | Liu_modified_ | GGGAAACCAA |
| | localGC_3_C | GAAUAAGAGCUCACUCGGAUAgAUUUGC |
| | MM | CACAGCAGAAGCCCACAC |
| 147 | Liu_modified_ | GGGAAACCAA |
| | totalGC_50_G | GAAGAAGAAUUUAUUUAGAUGCGCUUAU |
| | | CACAGCAGAAGCCCACAC |
| 148 | Liu_modified_ | GGGAAACCAA |
| | totalGC_50_C_ | GAAGAAGAAUUUAUUUAGAUGgGCUUAU |
| | MM | CACAGCAGAAGCCCACAC |
| 149 | AM254_Lbu_ | GGCCACCCCAAAAAUGAAGGGGACUAAAACAUAAGC |
| | mut_DR_modified_ | CCAUCUAAAUAAAU |
| | localGC_3_ | |
| | C | |
| 150 | AM255_Lbu_ | GGCCACCCCAAAAAUGAAGGGGACUAAAACAUAAGC |
| | mut_DR_modified_ | GCAUCUAAAUAAAU |
| | localGC_3_ | |
| | G | |
| 151 | AM256_Lbu_ | GGCCACCCCAAAAAUGAAGGGGACUAAAACGCAAAU |
| | mut_DR_modified_ | CUAUCCGAGUGAGC |
| | totalGC_50_ | |
| | C | |
| 152 | AM257_Lbu_ | GGCCACCCCAAAAAUGAAGGGGACUAAAACGCAAAU |
| | mut_DR_modified_ | GUAUCCGAGUGAGC |
| | totalGC_50_ | |
| | G | |
| 153 | AM261_WT_ | GGCCACCCCAAAAAUGAAGGGGACUAAAACUUCCUA |
| | del_crRNA_ | AACAAUCUAUACAG |
| | L452R_S_20 nt_ | |
| | MM19 | |
| 154 | AM262_Delta_ | GGCCACCCCAAAAAUGAAGGGGACUAAAACUUCCUA |
| | del_crRNA_ | AACAAUCUAUACCG |
| | L452R_S_20 nt_ | |
| | MM19 | |
| 155 | AM238_SARS_ | GAAATTAATACGACTCACTATAGGG |
| | CoV2_D80A_ | CAACTCAGGACTTGTTCTTACCTTTCTTTTCC |
| | fwd_Arizti- | |
| | Sanz | |
| 156 | AM239_SARS_ | AAGCAAAATAAACACCATCATTAAAT |
| | CoV2_D80A_ | |
| | rev_Arizti- | |
| | Sanz | |
| 157 | AM263_RPA_ | GAAATTAATACGACTCACTATAGGG |
| | fwd_Yang_ | CTTGATTCTAAGGTTGGTGGTAATTATAAT |
| | SARS_S452_T7 | |
| 158 | AM264_RPA_ | AAGGTTTGAGATTAGACTTCCTAAACAATC |
| | rev_Yang_ | |
| | SARS_S452 | |
| 159 | AM220_SARS_ | GAAATTAATACGACTCACTATAGGG |
| | CoV2_T478K_ | TTGAGAGAGATATTTCAACTGAAATCTATC |
| | fwd_Yang | |
| 160 | AM221_ | AGTGGGTTGGAAACCATATGATTGTAAAGG |
| | SARS_CoV2_ | |
| | T478K_rev_ | |
| | Yang | |
| 161 | crRNA-target | GGACCACCCCAAAAAUGAAGGGGACUAAAACACAAA |
| | RNA_(tgRNA) | UCUAUCUGAAUAAACUCUUCUUC |
| 162 | crRNA-anti-tag | GAAGAAGAGUUUAUUCAGAUAGAUUUGUCACAGCAG |
| | RNA_(A) | |
| 163 | crRNA-anti-tag | GAAGAAGAGUUUAUUCAGAUAGAUUUGUGUUUUAG |
| | RNA_(B) | U |

The application contains a Sequence Listing which has been submitted electronically in XML format and is hereby incorporated by reference in its entirety. Said. XML copy, created on Oct. 22, 2023, is named “1134-134 PCT.xml” and is 221,762 bytes in size. The sequence listing contained in this .XML file is part of the specification and is hereby incorporated by reference herein in its entirety.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A variant of a Cas protein, comprising one or more mutations at the HEPN 1 and HEPN2 interface of the Cas protein, wherein the one or more mutations modulate a specificity of the Cas protein against mismatches between a guide RNA and a target RNA.

2. The variant of claim 1, wherein the one or more mutation results in improved specificity against mismatches between a guide RNA and a target RNA.

3. The variant of claim 1, wherein the Cas protein is a Cas 13a protein.

4. The variant of claim 3, wherein the one or more mutations are selected from the group consisting of R377, N378, R963 and R973 of SEQ ID NO:14.

5. The variant of claim 4, wherein the variant comprises a mutation selected from the group consisting of R377A, N378A, R963A and R973A.

6. (canceled)

7. (canceled)

8. (canceled)

9. The variant of claim 5, wherein the Cas protein variant has the amino acid sequence selected from the group consisting of SEQ ID NOS: 15, 16, 17 and 18.

10. (canceled)

11. (canceled)

12. (canceled)

13. A guide RNA molecule, comprising:
a handle region; and
a spacer region consisting of 15-20 nucleotides directly linked to the 3′ end of the handle region, wherein the spacer region is located at the 3′ end of the guide RNA.

14. The guide RNA of claim 13, wherein the handle region comprises the sequence of SEQ ID NO: 19 or SEQ ID NO:20.

15. (canceled)

16. A polynucleic acid encoding the Cas protein variant of claim 1.

17. An expression vector, comprising:
a nucleic acid sequence encoding the Cas protein variant of claim 1; and
a regulatory sequence operably linked to the nucleic acid sequence.

18. A host cell, comprising the expression vector of claim 17.

19. A protein-RNA complex, comprising:
the Cas protein variant of claim 1;
a guide RNA; and
optionally a target RNA.

20. A method of making a variant of a Cas protein having the amino acid sequence of SEQ ID NO: 14, comprising:
introducing an expression vector comprising a nucleotide sequence encoding the variant Cas protein of the present application into a host cell;
culturing the host cell for a desired period of time to allow expression of the variant Cas protein; and
isolating the variant Cas protein from the host cell.

21. A method of detecting a single stranded target RNA in a sample, comprising: contacting the sample with (i) a guide RNA that hybridizes with the single stranded target RNA, and (ii) the Cas protein variant of claim 1; and measuring a signal produced by Cas protein-mediated RNA cleavage.

22. A method of detecting a single stranded target RNA in a sample, wherein the target RNA contains a single nucleotide polymorphism (SNP) in a target region, the method comprising:
contacting the sample with (i) a guide RNA comprising a handle region and a spacer region consisting of 15-40 nucleotides, wherein the spacer region is complementary to the target region in the target RNA, (ii) the Cas protein variant of claim 1, and (iii) a reporting construct capable of producing a signal upon interacting with a Cas protein-RNA complex that has Cas nuclease activity, wherein the Cas protein-RNA complex comprises the guide RNA, the Cas protein variant and the target RNA; and
measuring the signal from the reporting construct,
wherein detection of the signal from the reporting construct indicates the presence of the target RNA in the sample.

23. The method of claim 22, wherein the spacer region has a length consisting of 15-28 nucleotides.

24. The method of claim 22, wherein the spacer region has a length consisting of 15-20 nucleotides.

25. The method of claim 22, wherein the length of the spacer region is determined based on the GC content of nucleotide sequences around the SNP.

26. The method of claim 22, wherein the handle region comprises the sequence of SEQ ID NO:19 or SEQ ID NO: 20.

27. (canceled)

28. The method of claim 21, wherein the target RNA is a SARS virus RNA.

29. A target RNA detection kit comprising:
the Cas protein variant of claim 1;
a guide RNA that hybridizes with a target RNA; and
instructions for the use of the kit components.

30. (canceled)
